(19) United States
(12) Patent Application Publication
(10) Pub. No.: US 2006/0216336 A1
(43) Pub. Date: Sep. 28, 2006

(54) Transdermal therapeutic system for Parkinson's Disease

(76) Inventor: Hans-Michael Wolff, Monheim (DE)

Correspondence Address: JONES DAY, 222 EAST 41ST ST, NEW YORK, NY, 10017, US

(21) Appl. No.: 11/239,772

(22) Filed: Sep. 29, 2005

Related U.S. Application Data
(63) Continuation-in-part of application No. 10/139,894, filed on May 7, 2002.
(63) Continuation-in-part of application No. 10/140,096, filed on May 7, 2002.
(60) Provisional application No. 60/613,760, filed on Sep. 29, 2004.
(60) Provisional application No. 60/613,761, filed on Sep. 29, 2004.

(51) Int. Cl. A61K 31/381; A61F 13/02

(52) U.S. Cl. 424/448; 514/438

(57) ABSTRACT

The invention provides a transdermal therapeutic system (TTS) containing rotigotine as the active ingredient. The TTS is useful in the treatment of Parkinson's Disease because it induces a pharmacokinetic profile where the rotigotine plasma level is high and stable.

This application claims the benefit of U.S. Provisional Application Nos. 60/613,760 and 60/613,761, both filed Sep. 29, 2004, and U.S. Ser. No. 10/139,894, filed May 7, 2002, which claims the benefit of U.S. Provisional Application No. 60/363,638, filed Mar. 12, 2002 and U.S. Ser. No. 10/140,096, filed May 7, 2002 which claims the benefit of U.S. Provisional Application No. 60/363,655 filed Mar. 12, 2002. The entire contents of these applications are herein incorporated by reference.

Various references are cited through out the application to more fully describe the subject matter of the invention. These references are hereby incorporated in their entirety.

### FIELD OF THE INVENTION

The present invention relates to a skin patch (also known as a Transdermal Therapeutic System (TTS)) that delivers a sufficient amount of rotigotine, at a sufficient rate, to treat or alleviate the symptoms of Parkinson's Disease or Restless Legs Syndrome.

### BACKGROUND OF THE INVENTION

The dopaminergic system uses dopamine as a neurotransmitter and plays a key role in the pathogenesis of a number of diseases including Parkinson's Disease, Alzheimer's Disease, Huntington's Disease and Schizophrenia (Seigel, G., et al, Basic Neurochemistry, 4^{th} Ed., 1989, pp 815-822 and 864-866). The dopaminergic system has also been implicated with respect to depression (Dougherty, D., et al., J. Clin. Psychiatry, 1998; 59, Suppl 5:60-63), Restless Legs Syndrome (RLS) (Trenkwalder, C., et al. Lancet Neurol. August 2005;4(8):465-75.) and Periodic Limb Movement in Sleep PLMS (O'Brien, C., CNI Review Medical Journal, Spring 1999, Volume 10, No. 1).

Parkinson's Disease is primarily a disease of middle age and beyond, and it affects both men and women. The highest rate of occurrence of Parkinson's Disease is in the age group over 70 years old, where Parkinson's Disease exists in 1.5 to 2.5% of that population. The mean age at onset is between 58 and 62 years of age, and most patients develop Parkinson's Disease between the ages of 50 and 79. There are approximately 800,000 people in the United States alone with Parkinson's Disease.

Parkinson's Disease is believed to be primarily caused by the degeneration of dopaminergic neurons in the substantia nigra. This, in effect, results in loss of tonic dopamine secretion and dopamine-related modulation of neuronal activity in the caudate nucleus, and thus in a deficiency of dopamine in certain brain regions. The resulting imbalance of neurotransmitters acetylcholine and dopamine eventually results in disease related symptoms. Although usually regarded as a motor system disorder, Parkinson's Disease is now considered to be a more complex disorder that involves both motor and nonmotor systems. This debilitating disease is characterized by major clinical features including tremor, bradykinesia, rigidity, dyskinesia, gait disturbances, and speech disorders. In some patients, dementia may accompany these symptoms. Involvement of the autonomic nervous system may produce orthostatic hypotension, paroxysmal flushing, problems with thermal regulation, constipation, and loss of bladder and sphincter control. Psychological disorders such as loss of motivation and depression may also accompany Parkinson's Disease.

Early motor deficits of Parkinson's Disease can be traced to incipient degeneration of nigral dopamine-releasing cells. This neuronal degeneration produces a defect in the dopamineric pathway that connects the substantia nigra to the striatum. As the disease progresses, refractory motor, autonomic, and mental abnormalities may develop, which implies that there is progressive degeneration of striatal receptor mechanisms.

The clinical diagnosis of Parkinson's Disease is based on the presence of characteristic physical signs, e.g., tremor, rigidity of skeletal muscles, bradykinesia, impairment of postural reflexes, and gait distrubances. The disease is known to be gradual in onset, slowly progressive, and variable in clinical manifestation. Evidence suggests that the striatal dopamine content declines to 20% below levels found in age-matched controls before symptoms occur.

Treatment of Parkinson's Disease has been attempted with, inter alia, L-dopa, which still is the standard for the therapy of Parkinson's Disease. L-dopa is a compound that passes the blood-brain barrier as a precursor for dopamine and is then converted into dopamine in the brain. L-dopa improves the symptoms of Parkinson's Disease but may cause severe side effects. Moreover, the drug tends to lose its effectiveness after the first two to three years of treatment. After five to six years, only 25% to 50% of patients on L-dopa therapy maintain improvement.

Furthermore a major drawback of currently utilized therapies for Parkinson's Disease is the eventual manifestation of the “fluctuation syndrome,” which results in “all-or-none” conditions characterized by alternating “on” periods of mobility with dyskinesias and “off” periods with hypokinesia or akinesia. Patients who display unpredictable or erratic “on-off” phenomena with oral anti-Parkinson therapy have a predictable beneficial response to intravenous administration of L-dopa and other dopamine agonists, suggesting that fluctuations in plasma concentrations of drug are responsible for the “on-off” phenomena. The frequency of “on-off” fluctuations has also been improved by continuous infusions of the dopamine receptor agonists apomorphine and lisuride. However, this mode of administration is inconvenient. Therefore, other modes of administration providing a more stable plasma level would be beneficial.

As mentioned above, one treatment approach for Parkinson's Disease involves dopamine receptor agonists. Dopamine receptor agonists, sometimes also referred to as dopamine agonists, are substances which, while structurally different from dopamine, bind to dopamine receptors and trigger an effect which is comparable to that of dopamine. Due to the reduced side-effects, it is advantageous when the substances selectively bind to or interact with one or a subset of the known dopamine receptor subtypes. At present there are several classes of identified dopamine receptor subtypes, the most well characterized being the D1, D2, and D3 receptors.

One dopamine receptor agonist which has been used to treat the symptoms of Parkinson's Disease is a compound called rotigotine. Rotigotine is (6S)-6{propyl[2-(2-thienyl)ethyl]amino}-5,6,7,8-tetrahydro-1-naphthalenol (CAS No. 99755-59-6) having the structure:

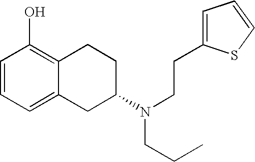

To date, various TTS's for the administration of rotigotine have been described. Published PCT Application No. WO 94/07468 discloses a transdermal therapeutic system containing rotigotine hydrochloride as active substance in a two-phase matrix which is essentially formed by a hydrophobic polymer material as the outer phase and a disperse hydrophilic phase contained therein and mainly containing the drug and hydrated silica. The silica enhances the maximum possible loading of the TTS with the hydrophilic salt. Moreover, the formulation disclosed in WO 94/07468 usually contains additional hydrophobic solvents, permeation-promoting substances, dispersing agents and, in particular, an emulsifier which is required to emulsify the aqueous solution of the active principle in the lipophilic polymer phase. A TTS prepared by using such a system has been tested in healthy subjects and Parkinson patients. The average drug plasma levels obtained by using this system were around 0.15 ng/mL with a 20 cm² patch containing 10 mg rotigotine hydrochloride. This level is considered too low to achieve a truly efficacious treatment or alleviation of the symptoms related to Parkinson's Disease.

Various further transdermal therapeutic systems have been described in Published PCT Application No. WO 99/49852. The TTS used in this patent application comprises a backing layer, inert with respect to the constituents of the matrix, a self-adhesive matrix layer containing an effective quantity of rotigotine or rotigotine hydrochloride and a protective film which is removed before use. The matrix system is composed of a non-aqueous polymer adhesive system, based on acrylate or silicone, with a solubility of rotigotine of at least 5% w/w. The matrix system is essentially free of inorganic silicate particles. In Examples 1 and 2 and in FIG. 1 of WO 99/49852, two transdermal therapeutic systems are compared. These are based on acrylate or silicone adhesives. FIG. 1 of WO 99/49852 shows that a silicone patch releases about the same amount of active principle through the skin as an acrylate patch. This has been demonstrated by the almost identical drug flux rates in an in vitro model, independent of the adhesive test system employed. Therefore an identical flux rate through human skin was expected.

It should be noted that the drug content of the silicone patch used in WO 99/49852 was lower than the drug content used in the acrylate patch. This merely reflects the difference in drug release capacity, however, in the respective polymeric silicone and acrylate adhesives used in Examples 1 and 2 of the published PCT application, respectively. While the acrylate system is able to dissolve more drug than the silicone system, silicone allows for a faster release of the drug to the skin. As these two effects compensate each other, it has been thought that the acrylate and the silicone system used in WO 99/49852 are about equivalent in the obtainable drug plasma levels and, hence, in therapeutic efficacy.

The shortcomings of the silicone formulation disclosed in WO 94/07468 have led to clinical tests (safety and pharmacokinetic studies) of only the acrylate-based TTS of Example 1 of WO 99/49852. The mean steady flux rate across human skin in vitro of this TTS amounted to 15.3 μg/cm²/h. Even the acrylate-based TTS, however, exhibited unsatisfactory plasma levels of rotigotine that are too low to allow for a really efficacious treatment of Parkinson's Disease. A 30 mg (20 cm²) patch only yielded a mean maximum plasma concentration of 0.12 ng/mL, while a 5 cm² patch containing 7.5 mg yielded a mean maximum plasma concentration of 0.068 ng/mL. Again, such values are too low to provide a real therapeutic progress in the treatment of Parkinson's Disease. In sum, neither the 20 cm² silicone patch disclosed in WO 94/07468 nor the 20 cm² acrylate patch disclosed in WO 99/49852 provided sufficient drug plasma levels to provide a satisfactory therapeutic effectiveness in the treatment of Parkinson's Disease.

The Restless Legs Syndrome (RLS) is a neurological disease that expresses itself as a false sensation in the legs accompanied by a strong kinetic urge. Symptoms of RLS include tingling, pulling, aching, itching, burning, cramps or pain, causing in the person concerned the irresistible urge to move. This disorder occurs most frequently when the person concerned is resting. It is particularly during the night's sleep that this sensory disorder with its attendant kinetic urge leads to restlessness and sleep interruptions. RLS can occur at any age but increases in frequency as persons grow older. It afflicts about 10% of the general population. Because of the nature of the symptoms, RLS is one of the most prevalent causes of sleep disturbances. In 20-40 year-olds, RLS accounts for 5%, in 40-60 year-olds for 20% and in those over 60 years of age for 35% of their sleeping-waking problem. Once the quality of sleep and thus of life of a patient has increasingly deteriorated due to RLS or the patient suffers from daytime somnolence, the need for therapy is indicated. Such need for therapy usually sets in at the age of 40-50 (U.S. Patent Application Publication No. 2004/0048779, paragraphs 0002 to 0005).

Therapy studies have revealed a diversity of results obtained in monotherapeutic treatments with dopamine agonists, opiates, benzodiazepines, carbamazepine, clonidine or levodopa (L-DOPA) in combination with a dopa decarboxylase inhibitor. The use of L-DOPA for treating RLS has been the subject of a particularly large number of papers. Long-term L-DOPA therapy leads to a clear mitigation of the disorder with an improved quality of sleep and life. The drawback of L-DOPA therapy, however, lies in the fact that in a great many patients its effectiveness tapers off and/or the RLS problem is shifted toward the morning hours (rebound) or the disorder is aggravated with the problem occurring even during the day (augmentation) (U.S. Patent Application Publication No. 2004/0048779, paragraph 0006).

Administration of rotigotine has been shown to lead to the suppression and reduction of RLS symptoms (U.S. Patent Application Publication No. 2004/0048779, paragraph 0012).

### SUMMARY OF THE INVENTION

Based on the results of human clinical trials involving both healthy subjects and early-stage Parkinson's patients the inventors have found that a transdermal therapeutic system (TTS) comprising a silicone matrix and rotigotine in its free base form produces a rotigotine pharmacokinetic profile with unexpectedly high plasma levels of rotigotine, a controlled release, substantially stable rotigotine blood plasma levels over time, and substantially uniform rotigotine plasma levels when the patch is placed at a variety of skin sites. For example, the inventors have demonstrated that a silicone-based TTS containing rotigotine in the free base form provides mean maximum drug plasma levels in the range of almost 0.5 ng/mL for a 20 cm² silicone patch containing 9 mg of rotigotine.

As such, the invention contemplates a treatment regimen that allows for repeated daily administration that achieves a steady state plasma concentration effective for alleviating symptoms of Parkinson's Disease. In particular, the methods of this invention produce continuous rotigotine plasma levels, which can be a more effective treatment than regimens producing pulsatile plasma levels.

The invention relates to methods for providing substantially controlled release of rotigotine and for inducing substantially steady-state rotigotine pharmacokinetic profiles over 24 hour period in a human patient in need thereof is provided, wherein the Cₘₐₓ of rotigotine is from about 0.14 ng/mL to about 1.54 ng/mL and the AUC_{O-t} is from about 3.3 ng/mL*h to about 32.2 ng/mL*h, said method comprising administering rotigotine to said human patient. In other aspects, the invention relates to methods for providing substantially controlled release of rotigotine and for inducing substantially steady-state rotigotine pharmacokinetic profiles over other and longer time periods, wherein the human patent suffers from Parkinson's Disease, Restless Legs Syndrome or another disease associated with the dopaminergic system. The invention also relates to methods for multiple administrations of rotigotine patches, and to methods for providing substantially controlled release of rotigotine and for inducing substantially steady-state rotigotine pharmacokinetic profiles by placing rotigotine skin patches at various skin sites. The methods of the invention encompass administration of rotigotine in various intervals effective to sustain a Cₘₐₓ at a level from about 0.14 ng/mL to about 1.54 ng/mL and the mean area under the curve (AUCₒ₋ₜ) at a level from about 3.3 ng/mL *h to about 32.2 ng/mL *h. The invention also relates to methods that involve rotating the transdermal application site on a daily basis, wherein the pharmacokinetic profiles remain unchanged.

In another aspect, the invention relates to a controlled release rotigotine formulation for transdermal administration to human patients, comprising from about 4 to about 20 mg rotigotine, where the formulation provides a mean maximum plasma concentration (Cₘₐₓ) of rotigotine from about 0.14 ng/mL to about 1.54 ng/mL and a mean area under the curve up to the last quantifiable concentration (AUC_{O-t}) from about 3.3 ng/mL*h to about 32.2 ng/mL*h.

In other, preferred aspects of the invention, the Cₘₐₓ of rotigotine induced by the formulation is from about 0.20 ng/mL to about 1.30 ng/mL; from about 0.30 ng/mL to about 1.20 ng/mL; from about 0.14 ng/mL to about 0.48 ng/mL; from about 0.37 ng/mL to about 0.75 ng/mL; or from about 0.84 ng/mL to about 1.54 ng/mL. In yet other preferred aspects of the invention, the induced Cₘₐₓ is about 0.31 ng/mL; about 0.56 ng/mL; or about 1.19 ng/mL.

In other aspects of the invention, the induced area-under-the-curve of the pharmacokinetic profile over time “t” (“AUC_{O-t}) is from about 4.0 ng/mL*h to about 30.0 ng/mL*h; from about 5.0 ng/mL*h to about 25.0 ng/mL*h; from about 3.3 ng/mL*h to about 8.9 ng/mL*h; from about 7 ng/mL*h to about 15.2 ng/mL*h; or from about 15.2 ng/mL*h to about 32.2 ng/mL*h. In other aspects, the induced AUC_{O-t}, is about 6.1 ng/mL*h; about 11.1 ng/mL*h; or about 23.7 ng/mL*h.

In another aspect of the invention, a method for treating Parkinson's Disease in human patient is provided, comprising administering rotigotine which, upon administration, provides a Cₘₐₓ of from about 0.14 ng/mL to about 1.54 ng/mL and wherein the AUC_{O-t} is from about 3.3 ng/mL*h to about 32.2 ng/mL*h.

In another aspect of the invention, a method for treating Restless Legs Syndrome in human patient is provided, comprising administering rotigotine which, upon administration, provides a Cₘₐₓ of from about 0.14 ng/mL to about 1.54 ng/mL and wherein the AUC_{O-t} is from about 3.3 ng/mL*h to about 32.2 ng/mL*h.

In one embodiment, the invention relates to a controlled release rotigotine formulation for transdermal administration to human patients, wherein said formulation gives the same pharmacokinetic profile regardless of where it is applied on the body of said human patient. In a preferred embodiment, the patient is suffering from Parkinson's disease. In another preferred embodiment, the patient is suffering from Restless Legs Syndrome.

### DESCRIPTION OF THE FIGURES

FIG. 1—Mean (±standard deviation) rotigotine plasma concentrations (in ng/mL) during and after single transdermal administration of 9.0 mg rotigotine with Patch A.

FIG. 2—Mean (±standard deviation) rotigotine plasma concentrations (in ng/mL) during and after single transdermal administration of 18.0 mg rotigotine with 2× Patch A.

FIG. 3—Mean (±standard deviation) rotigotine plasma concentrations (in ng/mL) during and after single transdermal administration of 33.48 mg rotigotine (state) with Patch B.

FIG. 4—Mean (±standard deviation) rotigotine plasma concentrations (in ng/mL) during and after multiple transdermal administration of 4.5 mg rotigotine with Patch C.

FIG. 5—Mean (±standard deviation) rotigotine plasma concentrations (in ng/mL) during and after last transdermal administration of 4.5 mg rotigotine with Patch C.

FIG. 6—Mean (±standard deviation) rotigotine plasma concentrations (in ng/mL) during and after single transdermal administration of 4.5 mg rotigotine with Patch D.

FIG. 7—Mean (±standard deviation) rotigotine plasma concentrations (in ng/mL) during and after single transdermal administration of 4.5 mg rotigotine with Patch C.

FIG. 8—Mean plasma concentrations versus time for each of the six application sites using combined data from Days 27 and 30 (after normalization by body weight and apparent dose).

FIG. 9—Plasma concentration over time for all patch application sites (after normalization by body weight and apparent dose).

FIG. 10—Arithmetic mean and standard deviation of the rotigotine plasma concentrations (ng/mL) during titration and maintenance phase.

### DETAILED DESCRIPTION OF THE PREFERRED EMBODIMENTS

I. Transdermal Therapeutic Systems

Transdermal therapeutic systems (TTS) of the present invention may be prepared using methods known in the art or as described in Published U.S. Patent Application Nos. US2003/0026830 and US2003/0027793 and U.S. Pat. No. 6,884,434, the disclosure of which as they relate to preparation of TTS's are incorporated by reference herein in their entirety.

In an embodiment, a TTS of the present invention is reservoir or matrix type transdermal system composed of one or more layers. In a further embodiment the TTS includes a backing layer and a liner layer that is removed prior to use.

In a preferred embodiment, a TTS of the present invention is a thin, matrix-type transdermal system composed of three layers:
(1) a flexible backing which is preferably siliconised on its inner side and is consisting of an aluminized polyester foil coated with a pigment-layer on the outer side or a transparent polyester film; and
(2) a self-adhesive drug matrix layer comprising of the active component rotigotine, ascorbyl palmitate, dl-alpha tocopherol, silicone adhesive, povidone, and sodium metabisulfite; and
(3) a protective liner, comprising of a transparent fluoropolymer-coated polyester film, which liner is removed prior to application.

A preferred process for making the TTS is described in U.S. Patent Application Publication No. US 2003/0026830 at paragraphs 38-42 and U.S. Patent Application Publication No. US 2003/0027793 at paragraphs 37-41, which are incorporated herein by this reference.

A preferred TTS of the present invention may contain from about 4 to about 20 mg of the rotigotine free base. In preferred embodiments, the TTS contains about 4.5 mg of the rotigotine free base, about 9 mg of the rotigotine free base, about 13.5 mg of the rotigotine free base, or about 18 mg of the rotigotine free base. In another preferred embodiment, the TTS contains 5-25% (w/w) rotigotine.

In a preferred embodiment of the present invention, the TTS is in the form of a patch. The release surface area of the patch may be from about 10 cm² to about 40 cm². In preferred embodiments of the present invention, the release surface area of the patch is about 10 cm², about 20 cm², about 30 cm², or about 40 cm².

A preferred embodiment of the invention utilizes a TTS containing one or more of the following: a pharmaceutically acceptable carrier (e.g., polyvinylpyrrolidone), sodium bisulfite, ascorbyl palmitate, DL-alpha-tocopherol, an amine resistant high tack silicone adhesive (e.g., BIO-PSA® Q7-4301; Dow Corning), and an amine resistant medium tack silicone adhesive (e.g., BIO-PSA® Q7-4201, Dow Corning). For example, a preferred 20 cm² patch TTS contains the components in the amounts described in Table 1.

TABLE 1

| Components | Amount (mg) |
|---|---|
| Rotigotine free base | 9.00 |
| Polyvinylpyrrolidone | 2.00 |
| Silicone BIO-PSA ® Q7-4301 | 44.47 |
| Silicone BIO-PSA ® Q7-4201 | 44.46 |
| Ascorbyl palmitate | 0.02 |
| DL-alpha tocopherol | 0.05 |
| Sodium metabisulfite | 0.0006 |

In a particularly preferred embodiment, the TTS comprises a self-adhesive matrix layer containing the free base of rotigotine in an amount effective for the treatment of the symptoms of Parkinson's Disease or restless legs syndrome (RLS), wherein the matrix is based on a silicone-based polymer adhesive system in which rotigotine free base is dispersed; a backing layer inert to the components of the matrix layer; and a protective foil or sheet covering the matrix layer to be removed prior to use. The TTS may also further comprise inert fillers to improve cohesion, e.g. polyvinylpyrrolidone. The TTS may also further comprise additives that facilitate a homogeneous dispersion of rotigotine particles in the form of hydrophilic polymers (e.g., polyvinylpyrrolidone, a copolymer of vinylpyrrolidone and vinylacetate, and a copolymer of ethylene and vinylacetate).

When the above-mentioned hydrophilic polymer is polyvinylpyrrolidone, the polyvinylpyrrolidone is present in the active substance-containing matrix layer in the form of insoluble particles at a concentration of 1.5-5% (w/w).

In one preferred embodiment, a TTS of the present invention is used to treat Parkinson's Disease or restless legs syndrome (RLS). As is used herein, the term “treatment” is meant to designate a treatment or alleviation of the symptoms of Parkinson's Disease or RLS, rather than a real causative treatment leading to a complete cure.

II. Rotigotine Pharmacokinetics and the TTS

### A. Pharmacokinetics

In an embodiment of the invention, the Cₘₐₓ of rotigotine induced by the formulation is from about 0.20 ng/mL to about 1.30 ng/mL; from about 0.30 ng/mL to about 1.20 ng/mL; from about 0.14 ng/mL to about 0.48 ng/mL; from about 0.37 ng/mL to about 0.75 ng/mL; or from about 0.84 ng/mL to about 1.54 ng/mL. In yet other preferred aspects of the invention, the induced Cₘₐₓ is about 0.31 ng/mL; about 0.56 ng/mL; or about 1.19 ng/mL.

In other aspects of the invention, the induced area-under-the-curve of the pharmacokinetic profile over time “t” (“AUC_{O-t}) is from about 4.0 ng/mL*h to about 30.0 ng/mL*h; from about 5.0 ng/mL*h to about 25.0 ng/mL*h; from about 3.3 ng/mL*h to about 8.9 ng/mL*h; from about 7 ng/mL*h to about 15.2 ng/mL*h; or from about 15.2 ng/mL*h to about 32.2 ng/mL*h. In other aspects, the induced AUC_{O-t}, is about 6.1 ng/mL*h; about 11.1 ng/mL*h; or about 23.7 ng/mL*h.

In another preferred embodiment, the TTS is used in a method for treating Parkinson's Disease in humans, comprising administering rotigotine which, upon administration, provides a Cₘₐₓ of from about 0.14 ng/mL to about 1.54 ng/mL and wherein the AUC_{O-t} is from about 3.3 ng/mL*h to about 32.2 ng/mL*h.

The invention contemplates a TTS used to administer 0.5 mg to 20 mg rotigotine over a 24 hour period.

In preferred embodiments, a TTS of the present invention is used to administer 2, 4, 6, or 8 mg rotigotine over a 24 hour period. In certain embodiments, the TTS used to deliver the aforementioned dosages contains, at the time of application, 4.5, 9, 13.5, or 18 mg rotigotine, respectively.

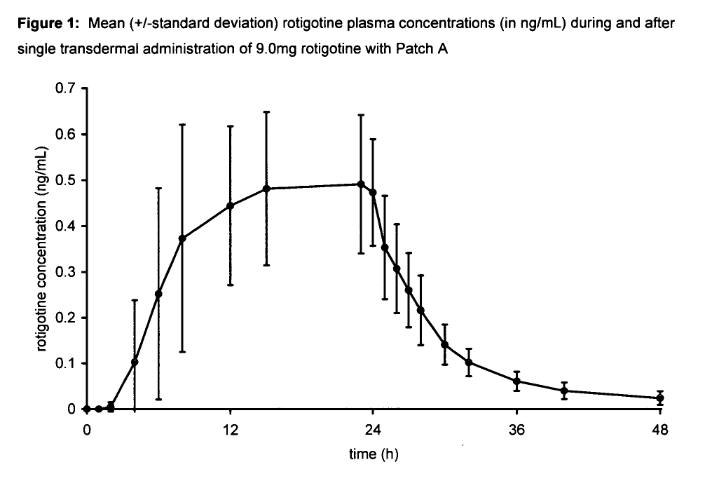

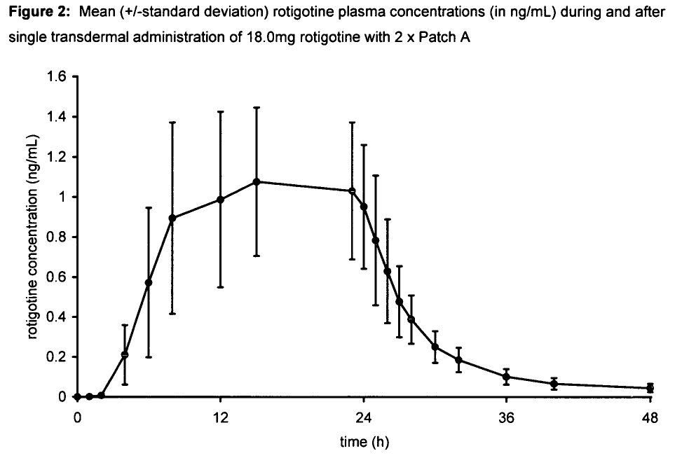

When applied once daily, a TTS of the present invention produces a sustained and relatively stable rotigotine plasma level. FIGS. 1-2 show a sustained and relatively stable rotigotine plasma level over a 24 hour period after single administration of a preferred patch (described in Example 1). In animal models of Parkinson's Disease, the presence of stable plasma levels of dopamine agonists such as rotigotine resulted in lower incidence of diskinesias compared to pulsatile plasma levels produced by intermittent administration. Chase, T. N., Drugs 55 Suppl. 1: 1-9 (1998); Stocchi, F. and Olanow, C. W., Neurology 62 (1 Suppl. 1): S56-S63 (2004).

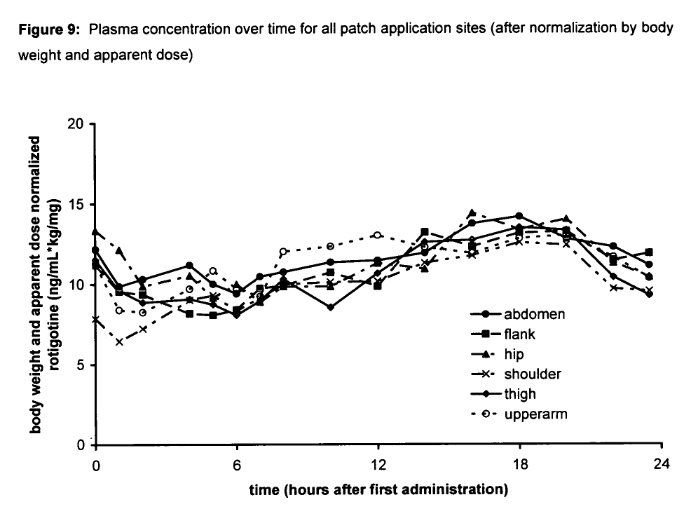

Rotigotine is released at a controlled rate following application of a TTS of the present invention to the skin. Approximately 45% of the rotigotine content of the TTS is released within 24 hours. Steady-state rotigotine plasma concentrations are reached after one to two days of transdermal administration and are maintained by once daily application of the TTS, where the TTS is worn by the patient for 24 hours. In the clinical trials of rotigotine effectiveness using neupro™, the mean trough plasma concentrations of rotigotine were stable over the six months of maintenance treatment. The bioavailability of rotigotine was similar across all application sites. FIG. 9 shows that the AUC_{O-t} and the Cₘₐₓ, for example, are comparable whether the TTS of the present invention is administered to the hip, shoulder, upper arm, thigh, abdomen or flank.

Rotigotine plasma levels have been determined in unconjugated blood samples or conjugated blood samples.

Exposure to rotigotine from daily application of the TTS of the present invention in healthy subjects and Parkinson's Disease patients exhibited a consistent exposure profile. Repeated daily administration resulted in stable plasma levels. After removal of the TTS, plasma levels decrease with an elimination half-life life of 5 to 7 hours.

Pharmacokinetic parameters observed after single dose or multiple dose application of a preferred TTS of the present invention to healthy subjects are summarized in Table 2.

TABLE 2

| Dose/24 hours | | | | |
|---|---|---|---|---|
| (TTS dimension) | | | AUC₀₋ₜ^{#1} | CL^{#1} |
| (n) | Design | Cₘₐₓ^{#1} | (ng/mL * h) | (L/min) |
| 4.5 mg | MD^{#2} | 0.31 ± 0.17 | 6.1 ± 2.8 | 8.1 ± 5.3 |
| (10 cm²) | | | | |
| (n = 29) | | | | |
| 9 mg | SD^{#3} | 0.56 ± 0.19 | 11.1 ± 4.1 | 8.0 ± 2.2 |
| (20 cm²) | | | | |
| (n = 13) | | | | |
| 18 mg | SD^{#3} | 1.19 ± 0.35 | 23.7 ± 8.5 | 7.5 ± 2.0 |
| (2 * 20 cm²) | | | | |
| (n = 11) | | | | |

^{#1}Mean ± SD
^{#2}Multiple Dose, see example 2
^{#3}Single Dose, see example 1
Cₘₐₓ is the mean maximum plasma concentration.
AUC₀₋ₜ is the mean area under the curve until the last quantifiable concentration.
CL is clearance.

### B. Preferred Embodiments

In a preferred embodiment of the present invention, the TTS contains a controlled release rotigotine formulation for transdermal administration to human patients, comprising from about 4 to about 20 mg rotigotine, said formulation resulting in a mean maximum plasma concentration (Cₘₐₓ) of rotigotine from about 0.14 ng/mL to about 1.54 ng/mL and a mean area under the curve up to the last quantifiable concentration (AUC_{O-t}) from about 3.3 ng/mL*h to about 32.2 ng/mL*h.

In a preferred embodiment of the present invention, the TTS contains a controlled release rotigotine formulation for transdermal administration to human patients, comprising from about 4.5 to about 18 mg rotigotine, said formulation providing a mean maximum plasma concentration (Cₘₐₓ) of rotigotine from about 0.14 ng/mL to about 1.54 ng/mL and a mean area under the curve up to the last quantifiable concentration (AUC_{O-t}) from about 3.3 ng/mL*h to about 32.2 ng/mL*h.

In still another preferred embodiment, the TTS is used in a method for inducing a steady-state rotigotine pharmacokinetic profile over a 24 hour period in a human patient suffering from Parkinson's Disease, wherein the Cₘₐₓ of rotigotine is from about 0.14 ng/mL to about 1.54 ng/mL and the AUC_{O-t} is from about 3.3 ng/mL*h to about 32.2 ng/mL*h, said method comprising administering rotigotine to said human patient.

In an embodiment, the invention relates to a method for treating Parkinson's Disease in a human patient, comprising administering to the patient a rotigotine formulation capable of providing a plasma concentration effective to alleviate the symptoms of Parkinson's Disease, wherein the Cₘₐₓ is from about 0.14 ng/mL to about 1.54 ng/mL and wherein the mean area under the curve (AUCₒ₋ₜ) is from about 3.3 ng/mL *h to about 32.3 ng/mL *h. In certain embodiments, the formulation is administered daily in 24 hour intervals.

In another embodiment, the invention relates to a method for treating Parkinson's Disease in a human patient, comprising administering to the patient a rotigotine formulation capable of maintaining a plasma concentration effective to alleviate the symptoms of Parkinson's Disease, wherein the Cₘₐₓ is sustained at a level from about 0.14 ng/mL to about 1.54 ng/mL and wherein the mean area under the curve (AUCₒ₋ₜ) is from about 3.3 ng/mL *h to about 32.2 ng/mL *h.

In yet another embodiment, the invention relates to a method of treating Parkinson's Disease in a human patient, comprising applying a transdermal therapeutic system (TTS) comprising rotigotine, wherein the TTS is capable of providing a plasma concentration effective to alleviate the symptoms of Parkinson's Disease, wherein the Cₘₐₓ is from about 0.14 ng/mL to about 1.54 ng/mL and wherein the mean area under the curve (AUCₒ₋ₜ) is from about 3.3 ng/mL *h to about 32.2 ng/mL *h.

In a further embodiment, the invention relates to a method of treating Parkinson's Disease in a human patient comprising applying one or more transdermal patches comprising an amount of rotigotine from 4 mg to 20 mg to the human patient; so as to produce in the human patient a mean maximum plasma concentration (Cₘₐₓ) of rotigotine effective to alleviate the symptoms of Parkinson's Disease in the human patient, wherein the Cₘₐₓ of rotigotine in the patient is sustained at a level from about 0.14 ng/mL to about 1.54 ng/mL and the last quantifiable concentration (AUCₒ₋ₜ) of the rotigotine in the patient is sustained at a level from about 3.3 ng/mL *h to about 32.2 ng/mL *h.

In a further embodiment, the invention relates to a method of treating Parkinson's Disease in a human patient comprising
a) applying one or more transdermal patches comprising an amount of rotigotine from 4 mg to 20 mg to the human patient;
b) removing the patch or patches of step a) and applying another patch or patches comprising an amount of rotigotine from 4 mg to 20 mg to the human patient at an interval so as to produce in the human patient a mean maximum plasma concentration (Cₘₐₓ) of rotigotine effective to alleviate the symptoms of Parkinson's Disease in the human patient; and
c) repeating step b) as required to sustain the Cₘₐₓ of rotigotine in the human patient at a level effective to alleviate the symptoms of Parkinson's Disease in the human patient wherein the Cₘₐₓ of rotigotine is sustained at a level from about 0.14 ng/mL to about 1.54 ng/mL.

In a preferred embodiment of the invention, the Cₘₐₓ of rotigotine in the human patient is sustained from 3 days to 28 weeks, from 1 to 7 days, from 1 to 6 weeks, for 7 weeks, from 8 to 28 weeks or for 28 weeks.

In another preferred embodiment of the invention, the patch or patches are removed and another patch or patches are applied daily, twice daily, weekly, twice weekly, monthly or twice monthly.

In other, preferred aspects of the invention, the Cₘₐₓ of rotigotine in the human patient is sustained at a level from about 0.20 ng/mL to about 1.30 ng/mL; from about 0.30 ng/mL to about 1.20 ng/mL; from about 0.14 ng/mL to about 0.48 ng/mL; from about 0.37 ng/mL to about 0.75 ng/mL; or from about 0.84 ng/mL to about 1.54 ng/mL. In yet other preferred aspects of the invention, the induced Cₘₐₓ is about 0.31 ng/mL; about 0.56 ng/mL; or about 1.19 ng/mL.

In one embodiment, the invention relates to a controlled release rotigotine formulation for transdermal administration to human patients, wherein said formulation is capable of providing a plasma concentration effective to alleviate the symptoms of Parkinson's Disease regardless of where it is applied on the body of said human patient. In a preferred embodiment, the patients are suffering from Parkinson's disease. In another preferred embodiment, the patients are suffering from restless legs syndrome. In still another embodiment, the patients are suffering from a disease related to the dopaminergic system.

In another embodiment, the invention relates to a method for inducing a steady-state rotigotine pharmacokinetic profile over a 24 hour period in a human patient in need thereof comprising administering rotigotine to said human patient, wherein the Cₘₐₓ of rotigotine is from about 0.14 ng/mL to about 1.54 ng/mL and the AUC_{O-t} is from about 3.3 ng/mL *h to about 32.2 ng/mL *h, wherein the method gives the same Cₘₐₓ and AUC_{O-t} regardless of where the rotigotine is administered to the body of the human patient.

In another embodiment, the invention relates to a method for treating Parkinson's Disease in a human patient, comprising administering to the patient over a 24 hr period a rotigotine formulation capable of providing a plasma concentration effective to alleviate the symptoms of Parkinson's Disease, wherein the Cₘₐₓ is from about 0.14 ng/mL to about 1.54 ng/mL and the AUC_{O-T} is from about 3.3 ng/mL *h to about 32.2 ng/mL *h.

In another embodiment, the invention relates to a method provides the same plasma concentration effective to alleviate the symptoms of Parkinson disease regardless of where the rotigotine is administered to the body of the human patient.

In yet another embodiment, the invention relates to a method of treating Parkinson's Disease in a human patient comprising applying one or more transdermal patches comprising an amount of rotigotine from 4 mg to 20 mg to the patient to provide a plasma concentration effective to alleviate the symptoms of Parkinson's Disease in the human patient, wherein the Cₘₐₓ is sustained at a level from about 0.14 ng/mL to about 1.54 ng/mL and the mean area under the curve (AUCₒ₋ₜ) of the rotigotine in the patient is from about 3.3 ng/mL *h to about 32.2 ng/mL *h.

In an embodiment of the invention, a single daily dose of rotigotine should be initiated and then increased in increments to an effective dose. In another embodiment, the dose is administered with a transdermal therapeutic system (TTS). In yet another embodiment, the TTS is applied once a day. In a further embodiment, the TTS should be applied at the same time every day. In another embodiment, the application site of the TTS should be moved on a daily basis, for example from the right side to the left side and from the upper body to the lower body.

In certain embodiments, the transdermal system is replaced every 48 hours preferably every 24 hours. The application site does not affect the pharmacokinetic profile. In non-limiting examples the TTS can be applies to the front of the abdomen, thigh, hip, flank, shoulder or upper arm. Preferably the TTS is moved on a daily basis, for example from the right side to the left side, from the upper body to the lower body. Preferable the TTS is not applied to the same site more than once every 7 days, 10 days, 14 days, 17 days or 21 days.

The present invention is illustrated by the following examples, without limiting the scope of the invention.

Abbreviations

As used above, and elsewhere herein, the following terms and abbreviations have the meanings defined below:
AUC_{O-t} : area under the curve from zero up to the last quantifiable concentration.
AUC(048): area under the curve from zero up to 48 hours after administration.
AUC_{O-t} : area under the curve from zero up to infinity calculated using the area under the curve after the first 24 hours (AUC₀₋₂₄) and extrapolating to infinity such that AUC_{O-inf}=AUC₀₋₂₄+plasma concentration at 24 hours/kₑₗ.
C_{trough}: measured trough plasma concentration.
CL: total body clearance.
Cₘₐₓ : maximum measured plasma concentration
C_{max,T}: maximum measured plasma concentration during a dose interval, T.
Cₘᵢₙ: minimum measured plasma concentration.
C_{min,T}: minimum measured plasma concentration during a dose interval T.
CV: coefficient of variation.
kₑₗ: rate constant of elimination.
LLQ: lower limit of quantification.
std: standard deviation swing: fluctuation of the plasma concentration calculated by (Cₘₐₓ−Cₘᵢₙ)/(0.5*Cₘₐₓ+0.5*Cₘᵢₙ)* 100%.
t_{tag}: lag time; elapsed time until onset of absorption.
tₘₐₓ: time of Cₘₐₓ.
tₘᵢₙ: time of Cₘᵢₙ.
(Site of administration: H=hip, S=shoulder, UA=upper arm, T=thigh, AB=abdomen, F=flank

### EXAMPLE 1

### Study Design and Subject Population

A single-center, open-label, single administration, three-way cross-over clinical trial was performed to assess the blood levels and comparative bioavailability of rotigotine from silicone and acrylic transdermal patches. The acrylic transdermal patches were made in accordance with the teachings of WO 99/49852. The silicone transdermal patches were made in accordance with the teachings of U.S. Patent Application Publication No. US 2003/0026830 at paragraphs 38-42, U.S. Patent Application Publication No. US 2003/0027793 at paragraphs 37-41 and U.S. Pat. No. 6,884,434, columns 5-6, Example 2 and comprised the following components:

Patch A

| Name of Ingredient | mg/20 cm² patch | |
|---|---|---|
| Rotigotine | 9.00 | |
| Silicone adhesive 4301 | 44.47 | |
| Silicone adhesive 4201 | 44.46 | |
| Providone | 2.00 | |
| Sodium metabisulfite | 0.0009 | |
| Ascorbyl palmitate | 0.02 | |
| Vitamin E (DL-α-tocopherol) | 0.05 | |
| Scotchpak 1109 (backing film) | 20 | cm² |

Patch B

| Name of Ingredient | mg/20 cm² patch | |
|---|---|---|
| Rotigotine HCl | 33.48 | |
| Sodium trisilicate | 19.2 | |
| Oleyl alcohol | 12 | |
| Vinylacetate-acrylate copolymer | 44.26 | |
| Eudragit E 100 | 11.06 | |
| Polyester (separator film) | 20 cm² | |
| Silicone adhesive 4301 (overlay) | 174.6 | |
| Silicone oil Q7 9120 (overlay) | 5.4 | |
| Hostaphan RN 15 backing film | 30 | cm² |

In a first period, a single silicone patch A was administered to each of 14 healthy male subjects (Caucasian race, aged 18-50 years) for a period of 24 hours. After a six day wash-out period, the same subjects were in randomized order administered either a single acrylic patch B for 24 hours in the second period followed another six day wash-out period and then administered two silicone patches A for 24 hours in the third period or administered two silicone patches A for 24 hours in the second period followed another six day wash-out period and then administered a single acrylic patch B for 24 hours in the third period. The silicone patches, had a rotigotine content of 9 mg /20 cm² and the acrylic patches had a rotigotine content of 33.48 mg/20 cm².

During each study period blood samples for the analysis of rotigotine were taken before patch application and at 1 h, 2 h, 4 h, 6 h, 8 h, 12 h, 15 h, 23 h, 24 h, 25 h, 26 h, 27 h, 28 h, 30 h, 32 h, 36 h, 40 h and 48 h after first patch application.

To characterize the pharmacokinetics of rotigotine after administration of rotigotine patches in healthy volunteers, the maximum plasma concentration (Cₘₐₓ) and the corresponding timepoint (tₘₐₓ) were taken and the data was separated by formulation (and dose). For each sequence of plasma concentrations the AUC was calculated using the trapezoidal rule. AUC₍₀₋₁₎ represents the AUC from patch administration up to the last quantifiable plasma concentration (e.g., if the concentration dropped to below quantifiable levels in less than 48 hours) whereas AUC₍₀₋₄₈₎ presents the AUC from patch administration to the last sampling point, 48 h after start of administration. The total body clearance was calculated from the individual apparent dose and the corresponding AUC. AUC was the individual area under the concentration time curve extrapolated to infinity: AUC=AUC(O-t)+C(t)/kel, where C(t) is the last quantificable plasma concentration.

Plasma Concentrations of Rotigotine

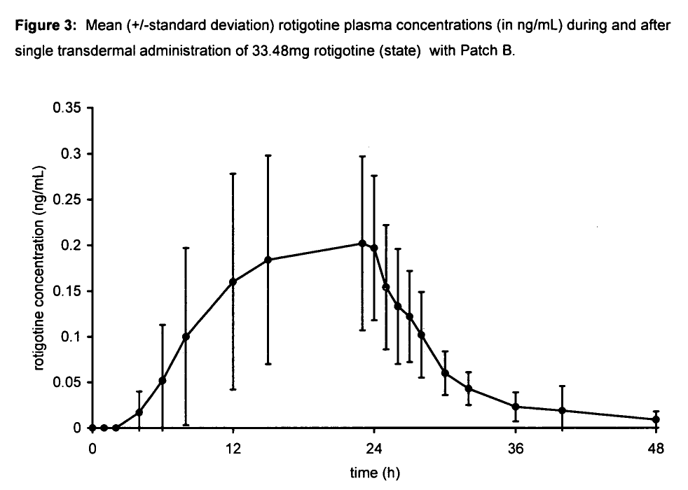

Data for the rotigotine plasma concentrations and pharmacokinetic parameters measured during this clinical trial for the silicone patches are provided in Tables 3, 4, 5, and 6. Data for rotigotine plasma concentration for the acrylic patch are provided in Tables 7 and 8. FIGS. 1 and 2 illustrate the arithmetic mean of rotigotine plasma concentration for single dose administration of the silicone patch. FIG. 3 illustrates the arithmetic mean of rotigotine plasma concentration for single dose administration of the acrylic patch.

TABLE 3
Individual rotigotine plasma concentrations (in ng/mL) during and after single
transdermal administration of 9.0 mg rotigotine with Patch A (n.s. = no sample).

| subj. | time [h] | | | | | | | | | |
|---|---|---|---|---|---|---|---|---|---|---|
| no. | 0 | 1 | 2 | 4 | 6 | 8 | 12 | 15 | 23 | 24 |
| 1 | 0 | 0 | 0 | 0 | 0.0783 | 0.166 | 0.297 | 0.381 | 0.456 | 0.406 |
| 2 | 0 | 0 | 0 | 0.119 | 0.211 | 0.467 | 0.5 | 0.537 | 0.606 | 0.459 |
| 3 | 0 | 0 | 0 | 0.0107 | 0.06 | 0.162 | 0.293 | 0.34 | 0.367 | 0.42 |
| 4 | 0 | 0 | 0 | 0.0675 | 0.462 | 0.648 | 0.685 | 0.831 | 0.671 | 0.645 |
| 5 | 0 | 0 | 0 | 0.0717 | 0.241 | 0.348 | 0.496 | 0.526 | 0.721 | 0.612 |
| 6 | 0 | 0 | 0 | 0.0616 | 0.156 | 0.334 | 0.446 | 0.478 | 0.586 | 0.564 |
| 7 | 0 | 0 | 0 | 0.0223 | 0.0845 | 0.172 | 0.239 | 0.306 | 0.318 | 0.319 |
| 8 | 0 | 0 | 0 | 0.017 | 0.126 | 0.223 | 0.278 | 0.323 | 0.449 | 0.441 |
| 9 | 0 | 0 | 0.0265 | 0.156 | 0.304 | 0.374 | 0.461 | 0.434 | 0.511 | 0.466 |
| 10 | 0 | 0 | 0 | 0.0396 | 0.139 | 0.202 | 0.379 | 0.311 | 0.235 | 0.295 |
| 11 | 0 | 0 | 0.178 | 0.862 | 1.07 | 1.04 | 0.945 | 0.764 | 0.194 | n.s. |
| 12 | 0 | 0 | 0 | 0 | 0.0743 | 0.165 | 0.352 | 0.433 | 0.357 | 0.379 |
| 15 | 0 | 0 | 0.0238 | 0.376 | 0.867 | 0.985 | 0.849 | 0.748 | 0.668 | 0.647 |
| 23 | 0 | 0 | 0.0103 | 0.4 | 0.472 | 0.602 | 0.492 | 0.608 | 0.434 | 0.493 |

| subj. | time [h] | | | | | | | | |
|---|---|---|---|---|---|---|---|---|---|
| no. | 25 | 26 | 27 | 28 | 30 | 32 | 36 | 40 | 48 |
| 1 | 0.289 | 0.245 | 0.228 | 0.168 | 0.15 | 0.103 | 0.0799 | 0.0376 | 0.0179 |
| 2 | 0.325 | 0.312 | 0.287 | 0.235 | 0.16 | 0.119 | 0.053 | 0.0476 | 0.0289 |
| 3 | 0.285 | 0.296 | 0.182 | 0.14 | 0.0798 | 0.0608 | 0.04 | 0.028 | 0.0137 |
| 4 | 0.489 | 0.426 | 0.335 | 0.277 | 0.168 | 0.143 | 0.0884 | 0.0521 | 0.035 |
| 5 | 0.513 | 0.422 | 0.382 | 0.361 | 0.23 | 0.149 | 0.0927 | 0.0682 | 0.0535 |
| 6 | 0.382 | 0.317 | 0.292 | 0.275 | 0.18 | 0.125 | 0.0826 | 0.0664 | 0.0355 |
| 7 | 0.255 | 0.229 | 0.218 | 0.186 | 0.132 | 0.0914 | 0.0467 | 0.0281 | 0.0103 |
| 8 | 0.282 | 0.281 | 0.237 | 0.193 | 0.145 | 0.0981 | 0.0569 | 0.0386 | 0.0187 |
| 9 | 0.389 | 0.297 | 0.258 | 0.217 | 0.129 | 0.0974 | 0.0544 | 0.0257 | 0.0187 |
| 10 | 0.186 | 0.162 | 0.13 | 0.107 | 0.0582 | 0.0386 | 0.024 | 0.0163 | 0 |
| 11 | n.s. | n.s. | n.s. | n.s. | n.s. | n.s. | n.s. | n.s. | n.s. |
| 12 | 0.306 | 0.267 | 0.236 | 0.178 | 0.131 | 0.103 | 0.0617 | 0.0288 | 0.0151 |
| 15 | 0.58 | 0.516 | 0.413 | 0.328 | 0.166 | 0.109 | 0.0752 | 0.0593 | 0.0464 |
| 23 | 0.311 | 0.215 | 0.177 | 0.146 | 0.107 | 0.0868 | 0.0379 | 0.0182 | 0.0162 |

Dimension concentration = [ng/ml]

TABLE 4
Parameters of model independent pharmacokinetics of
rotigotine during and after single transdermal administration
of 9.0 mg rotigotine with Patch A

| subj. | Cₘₐₓ | tₘₐₓ | AUC(0-48) | AUC(0-t) | t |
|---|---|---|---|---|---|
| 1 | 0.456 | 23 | 8.4874 | 8.4874 | 48 |
| 2 | 0.606 | 23 | 12.5172 | 12.5172 | 48 |
| 3 | 0.42 | 24 | 7.2922 | 7.2922 | 48 |
| 4 | 0.831 | 15 | 16.8722 | 16.8722 | 48 |
| 5 | 0.721 | 23 | 13.9144 | 13.9144 | 48 |
| 6 | 0.586 | 23 | 11.8375 | 11.8375 | 48 |
| 7 | 0.319 | 24 | 6.9149 | 6.9149 | 48 |
| 8 | 0.449 | 23 | 8.3738 | 8.3738 | 48 |
| 9 | 0.511 | 23 | 11.11405 | 11.11405 | 48 |
| 10 | 0.379 | 12 | 6.4172 | 6.352 | 40 |
| 12 | 0.433 | 15 | 8.3696 | 8.3696 | 48 |
| 15 | 0.985 | 8 | 19.7174 | 19.7174 | 48 |
| 23 | 0.608 | 15 | 12.79945 | 12.79945 | 48 |
| Min | 0.319 | 8 | 6.4172 | 6.352 | 40 |
| Max | 0.985 | 24 | 19.7174 | 19.7174 | 48 |
| Med | 0.511 | 23 | 11.11405 | 11.11405 | 48 |
| Mean | 0.562 | 19.308 | 11.125 | 11.12 | 47.385 |
| SD | 0.191 | 5.498 | 4.048 | 4.054 | 2.219 |

Dimension: Cₘₐₓ [ng/ml] tₘₐₓ, t [h] AUC [ng/ml h]

TABLE 5
Individual rotigotine plasma concentrations (in ng/mL) during and after single
transdermal administration of 18.0 mg rotigotine with 2 × Patch A

| subj. | time [h] | | | | | | | | | |
|---|---|---|---|---|---|---|---|---|---|---|
| no. | 0 | 1 | 2 | 4 | 6 | 8 | 12 | 15 | 23 | 24 |
| 1 | 0 | 0 | 0 | 0.0474 | 0.228 | 0.48 | 0.845 | 1.11 | 0.992 | 0.814 |
| 2 | 0 | 0 | 0.0185 | 0.348 | 0.976 | 1.21 | 1.53 | 1.35 | 1.26 | 1.19 |
| 4 | 0 | 0 | 0.0165 | 0.345 | 0.955 | 1.71 | 1.7 | 1.56 | 1.54 | 1.19 |
| 5 | 0 | 0 | 0 | 0.111 | 0.413 | 0.805 | 1.07 | 1.19 | 1.25 | 1.36 |
| 6 | 0 | 0 | 0 | 0.228 | 0.54 | 1.13 | 1.34 | 1.55 | 1.54 | 1.34 |
| 7 | 0 | 0 | 0 | 0.169 | 0.438 | 0.772 | 0.841 | 0.819 | 0.801 | 0.614 |
| 8 | 0 | 0 | 0 | 0.01 | 0.0761 | 0.193 | 0.31 | 0.46 | 0.681 | 0.501 |
| 9 | 0 | 0 | 0.011 | 0.377 | 0.806 | 1.22 | 1.2 | 1.33 | 1.15 | 1.2 |
| 10 | 0 | 0 | 0.0277 | 0.321 | 0.565 | 0.864 | 0.705 | 0.678 | 0.593 | 0.732 |
| 12 | 0 | 0 | 0 | 0 | 0.0939 | 0.17 | 0.39 | 0.683 | 0.655 | 0.803 |
| 23 | 0 | 0.0116 | 0 | 0.356 | 1.2 | 1.28 | 0.929 | 1.11 | 0.866 | 0.717 |

| | |
|---|---|
| subj. | time [h] |

| no. | 25 | 26 | 27 | 28 | 30 | 32 | 36 | 40 | 48 |
|---|---|---|---|---|---|---|---|---|---|
| 1 | 0.613 | 0.567 | 0.447 | 0.448 | 0.289 | 0.228 | 0.112 | 0.0843 | 0.0432 |
| 2 | 1.09 | 1.03 | 0.684 | 0.494 | 0.354 | 0.235 | 0.127 | 0.0992 | 0.0755 |
| 4 | 1.16 | 0.687 | 0.558 | 0.45 | 0.323 | 0.205 | 0.13 | 0.0847 | 0.0592 |
| 5 | 1.35 | 0.89 | 0.603 | 0.518 | 0.358 | 0.277 | 0.166 | 0.113 | 0.0702 |
| 6 | 1.03 | 1.05 | 0.832 | 0.578 | 0.304 | 0.254 | 0.14 | 0.091 | 0.0725 |
| 7 | 0.625 | 0.616 | 0.45 | 0.344 | 0.208 | 0.141 | 0.0809 | 0.0428 | 0.0238 |
| 8 | 0.38 | 0.366 | 0.266 | 0.254 | 0.156 | 0.112 | 0.0554 | 0.0519 | 0.0304 |
| 9 | 0.808 | 0.567 | 0.462 | 0.402 | 0.205 | 0.181 | 0.112 | 0.0552 | 0.0368 |
| 10 | 0.496 | 0.377 | 0.285 | 0.196 | 0.136 | 0.0804 | 0.0407 | 0.0218 | 0.0235 |
| 12 | 0.468 | 0.367 | 0.325 | 0.302 | 0.19 | 0.155 | 0.0715 | 0.04 | 0.021 |
| 23 | 0.598 | 0.405 | 0.334 | 0.287 | 0.225 | 0.165 | 0.0724 | 0.0452 | 0.0419 |

Dimension concentration = [ng/ml]

TABLE 6
Parameters of model independent pharmacokinetics of
rotigotine during and after single transdermal administration
of 18.0 mg rotigotine. with 2 × Patch A

| subj. | Cₘₐₓ | tₘₐₓ | AUC(0-48) | AUC(0-t) | t |
|---|---|---|---|---|---|
| 1 | 1.11 | 15 | 21.0189 | 21.0189 | 48 |
| 2 | 1.53 | 12 | 32.30895 | 32.30895 | 48 |
| 4 | 1.71 | 8 | 36.01075 | 36.01075 | 48 |
| 5 | 1.36 | 24 | 27.5278 | 27.5278 | 48 |
| 6 | 1.55 | 15 | 32.956 | 32.956 | 48 |
| 7 | 0.841 | 12 | 18.9181 | 18.9181 | 48 |
| 8 | 0.681 | 23 | 10.6273 | 10.6273 | 48 |
| 9 | 1.33 | 15 | 28.2519 | 28.2519 | 48 |
| 10 | 0.864 | 8 | 16.35535 | 16.35535 | 48 |
| 12 | 0.803 | 24 | 12.6378 | 12.6378 | 48 |
| 23 | 1.28 | 8 | 24.375 | 24.375 | 48 |
| Min | 0.681 | 8 | 10.6273 | 10.6273 | 48 |
| Max | 1.71 | 24 | 36.01075 | 36.01075 | 48 |
| Med | 1.28 | 15 | 24.375 | 24.375 | 48 |
| Mean | 1.187 | 14.909 | 23.726 | 23.726 | 48 |
| SD | 0.349 | 6.252 | 8.511 | 8.511 | 0 |

Dimension: Cₘₐₓ [ng/ml] tₘₐₓ, t [h] AUC [ng/ml h]

TABLE 7
Individual rotigotine plasma concentrations (in ng/mL) during and after single
transdermal administration of 33.48 mg rotigotine with Patch B

| subj. | time [h] | | | | | | | | | |
|---|---|---|---|---|---|---|---|---|---|---|
| no. | 0 | 1 | 2 | 4 | 6 | 8 | 12 | 15 | 23 | 24 |
| 1 | 0 | 0 | 0 | 0 | 0.0215 | 0.0612 | 0.13 | 0.143 | 0.158 | 0.161 |
| 2 | 0 | 0 | 0 | 0.0111 | 0.0292 | 0.0491 | 0.165 | 0.233 | 0.281 | 0.29 |
| 4 | 0 | 0 | 0 | 0.043 | 0.197 | 0.326 | 0.418 | 0.437 | 0.348 | 0.264 |
| 5 | 0 | 0 | 0 | 0 | 0.0243 | 0.0617 | 0.181 | 0.237 | 0.274 | 0.277 |
| 6 | 0 | 0 | 0 | 0.0137 | 0.0421 | 0.109 | 0.221 | 0.267 | 0.366 | 0.341 |
| 7 | 0 | 0 | 0 | 0 | 0.0185 | 0.0403 | 0.0946 | 0.114 | 0.114 | 0.117 |
| 8 | 0 | 0 | 0 | 0 | 0 | 0.0139 | 0.0391 | 0.0494 | 0.159 | 0.193 |
| 9 | 0 | 0 | 0 | 0.0107 | 0.0241 | 0.0504 | 0.0797 | 0.109 | 0.137 | 0.157 |
| 10 | 0 | 0 | 0 | 0.0117 | 0.0302 | 0.0821 | 0.081 | 0.126 | 0.096 | 0.0919 |
| 12 | 0 | 0 | 0 | 0 | 0 | 0.0116 | 0.0299 | 0.0443 | 0.112 | 0.12 |
| 15 | 0 | 0 | 0 | 0.0715 | 0.143 | 0.248 | 0.339 | 0.298 | 0.23 | 0.205 |
| 23 | 0 | 0 | 0 | 0.043 | 0.0889 | 0.149 | 0.142 | 0.156 | 0.143 | 0.147 |

| | |
|---|---|
| subj. | time [h] |

| no. | 25 | 26 | 27 | 28 | 30 | 32 | 36 | 40 | 48 |
|---|---|---|---|---|---|---|---|---|---|
| 1 | 0.128 | 0.111 | 0.11 | 0.0942 | 0.0628 | 0.0459 | 0.0337 | 0.0978 | 0.0126 |
| 2 | 0.192 | 0.189 | 0.169 | 0.163 | 0.0805 | 0.059 | 0.0336 | 0.0246 | 0.021 |
| 4 | 0.172 | 0.145 | 0.123 | 0.12 | 0.07 | 0.0439 | 0.0228 | 0.0162 | 0.0107 |
| 5 | 0.234 | 0.184 | 0.179 | 0.177 | 0.0887 | 0.0691 | 0.0425 | 0.022 | 0.0145 |
| 6 | 0.312 | 0.287 | 0.222 | 0.171 | 0.105 | 0.0734 | 0.0559 | 0.0296 | 0.0231 |
| 7 | 0.112 | 0.108 | 0.0921 | 0.083 | 0.05 | 0.033 | 0.017 | 0.0104 | 0 |
| 8 | 0.119 | 0.0849 | 0.0789 | 0.0615 | 0.0462 | 0.0311 | 0.0188 | 0.0103 | 0.0116 |
| 9 | 0.139 | 0.0911 | 0.0842 | 0.0679 | 0.0445 | 0.0421 | 0.0182 | 0 | 0 |
| 10 | 0.0587 | 0.0662 | 0.0673 | 0.0441 | 0.0232 | 0.021 | 0 | 0 | 0 |
| 12 | 0.1 | 0.0757 | 0.0768 | 0.0619 | 0.0553 | 0.0317 | 0.0134 | 0 | 0 |
| 15 | 0.171 | 0.152 | 0.164 | 0.117 | 0.0687 | 0.0445 | 0.021 | 0.0171 | 0.0129 |
| 23 | 0.115 | 0.104 | 0.0961 | 0.0608 | 0.0296 | 0.0163 | 0 | 0 | 0 |

Dimension concentration = [ng/ml]

TABLE 8
Parameters of model independent pharmacokinetics of
rotigotine during and after single transdermal administration
of 33.48 mg rotigotine with Patch B

| subj. | Cₘₐₓ | tₘₐₓ | AUC(0-48) | AUC(0-t) | t |
|---|---|---|---|---|---|
| 1 | 0.161 | 24 | 3.8657 | 3.8657 | 48 |
| 2 | 0.29 | 24 | 5.1399 | 5.1399 | 48 |
| 4 | 0.437 | 15 | 8.2774 | 8.2774 | 48 |
| 5 | 0.277 | 24 | 5.2879 | 5.2879 | 48 |
| 6 | 0.366 | 23 | 6.6699 | 6.6699 | 48 |
| 7 | 0.117 | 24 | 2.512 | 2.4704 | 40 |
| 8 | 0.193 | 24 | 2.1029 | 2.1029 | 48 |
| 9 | 0.157 | 24 | 2.577 | 2.5406 | 36 |
| 10 | 0.126 | 15 | 2.19825 | 2.15625 | 32 |
| 12 | 0.12 | 24 | 1.61175 | 1.58495 | 36 |
| 15 | 0.339 | 12 | 6.4101 | 6.4101 | 48 |
| 23 | 0.156 | 15 | 3.3707 | 3.3381 | 32 |
| Min | 0.117 | 12 | 1.61175 | 1.58495 | 32 |
| Max | 0.437 | 24 | 8.2774 | 8.2774 | 48 |
| Med | 0.177 | 24 | 3.6182 | 3.6019 | 48 |
| Mean | 0.228 | 20.667 | 4.169 | 4.154 | 42.667 |
| SD | 0.109 | 4.812 | 2.154 | 2.167 | 6.893 |

Dimension: Cₘₐₓ [ng/ml] tₘₐₓ, t [h] AUC [ng/ml h]

### EXAMPLE 2

Study Design and Subject Population

A single-center, open-label, multiple dose clinical trial was performed to assess the pharmacokinetics of a rotigotine transdermal patch during 14 days of once-daily patch administration to 30 healthy male volunteers. The subjects were treated for two days with placebo patches and then either with placebo or rotigotine patches for 14 days (i.e., days 13-16). The silicone transdermal patches were made in accordance with the teachings of U.S. Patent Application Publication No. US 2003/0026830 at paragraphs 38-42, U.S. Patent Application Publication No. US 2003/0027793 at paragraphs 37-41 and U.S. Pat. No. 6,884,434, columns 5-6, Example 2 and comprised the following layers and components:

Patch C

| Name of Ingredient | mg/10 cm² patch | |
|---|---|---|
| Rotigotine | 4.50 | |
| Silicone adhesive 4301 | 22.24 | |
| Silicone adhesive 4201 | 22.23 | |
| Providone | 1.00 | |
| Sodium metabisulfite | 0.00045 | |
| Ascorbyl palmitate | 0.010 | |
| Vitamin E (DL-α-tocopherol) | 0.025 | |
| Scotchpak 1109 (backing film) | 10 | cm² |

The silicone patches had a rotigotine content of 4.5 mg/10 cm².

During the study blood samples for the analysis of rotigotine were taken before patch administration and at 1, 2, 4, 6, 12, 24, 48, 72, 96, 120, 144, 168, 192, 216, 240, 264, 288, 312, 316, 320, 324, 336, 337, 338, 339, 340, 342, 344, 350, 360, 372, and 384 hours after first patch administration.

To characterize the pharmacokinetics of rotigotine after multiple dose administration of rotigotine patches in healthy volunteers the maximum plasma concentration (Cₘₐₓ) and the corresponding timepoint (tₘₐₓ) were taken and the data separated by subject. For each time sequence of plasma concentrations the AUC was calculated using the trapezoidal rule. AUC₍₃₁₂₋₃₃₆₎ represents the AUC within the dose interval of 24 hours under steady state administration.

Plasma Concentrations of Rotigotine

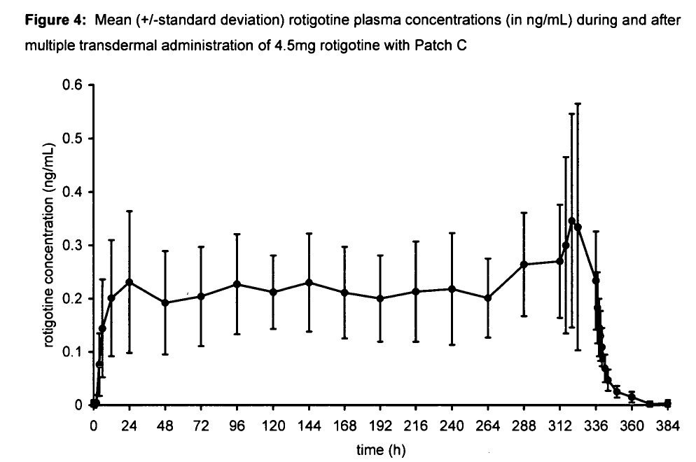

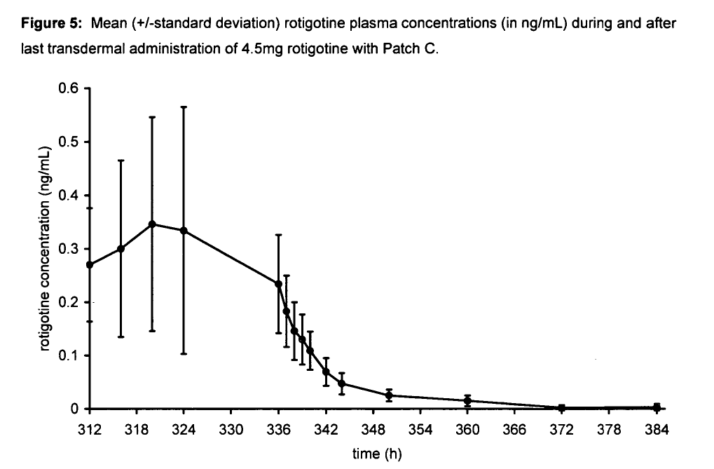

Data for the rotigotine plasma concentrations and pharmacokinetic parameters measured during this trial are provided in Tables 9 and 10. FIGS. 4 and 5 illustrate the arithmetic mean of rotigotine plasma concentration during and after multiple patch administration.

TABLE 9
Individual rotigotine plasma concentrations (in ng/mL) during and after multiple
transdermal administration of 4.5 mg rotigotine with Patch C.

| subj. | time [h] | | | | | | | | | | | | | | | | |
|---|---|---|---|---|---|---|---|---|---|---|---|---|---|---|---|---|---|
| no. | 0 | 1 | 2 | 4 | 6 | 12 | 24 | 48 | 72 | 96 | 120 | 144 | 168 | 192 | 216 | 240 | 264 |
| 01 | 0 | 0 | 0 | .0474 | .066 | .151 | .155 | .191 | .153 | .154 | .175 | n.s. | n.s. | n.s. | n.s. | n.s. | n.s. |
| 02 | 0 | 0 | 0 | .111 | .269 | .383 | .36 | .335 | .324 | .414 | .297 | .333 | .234 | .36 | .264 | .269 | .279 |
| 03 | 0 | 0 | 0 | .0577 | .13 | .184 | .206 | .237 | .2 | .241 | .204 | .233 | .27 | .212 | .196 | .208 | .241 |
| 04 | 0 | 0 | 0 | .0404 | .0586 | .0993 | .145 | .212 | .101 | .203 | .153 | .171 | .155 | .179 | .129 | .159 | .12 |
| 05 | 0 | 0 | 0 | .0398 | .134 | .122 | .26 | .181 | .273 | .271 | .265 | .226 | .218 | .143 | .126 | .194 | .191 |
| 06 | 0 | 0 | 0 | 0 | .0186 | .0855 | .118 | .0998 | .0986 | .111 | .197 | .116 | .129 | .146 | .141 | .132 | .144 |
| 07 | .0135 | 0 | .0519 | .149 | .174 | .221 | .194 | .18 | .158 | .139 | .174 | .183 | .146 | .157 | .184 | .179 | .151 |
| 08 | 0 | 0 | 0 | .0829 | .222 | .29 | .566 | .372 | .302 | .363 | .279 | .338 | .362 | .281 | .309 | .42 | .254 |
| 09 | 0 | 0 | 0 | .0683 | .157 | .24 | .252 | .214 | .184 | .297 | .237 | .247 | .265 | .28 | .247 | .294 | .323 |
| 10 | 0 | 0 | 0 | .0985 | .17 | .217 | .25 | .184 | .252 | .208 | .296 | .198 | .244 | .214 | .288 | .348 | .269 |
| 11 | .0179 | 0 | 0 | .0135 | .0255 | .0411 | .0437 | .0619 | .0662 | .146 | .0586 | .0995 | .046 | .0716 | .076 | .0705 | .132 |
| 12 | 0 | 0 | 0 | .208 | .305 | .375 | .22 | .0421 | .338 | .175 | .214 | .387 | .256 | .152 | .386 | .123 | .11 |
| 13 | 0 | 0 | 0 | .0128 | .0516 | .0842 | .144 | .152 | .114 | .156 | .199 | .256 | .301 | .284 | .242 | .168 | .191 |
| 14 | 0 | 0 | 0 | .0229 | .0662 | .113 | .0873 | .0927 | .0821 | .0873 | .141 | .105 | .151 | .126 | .121 | .168 | .139 |
| 15 | 0 | 0 | .0117 | .108 | .164 | .226 | .184 | .209 | .185 | .339 | .338 | .291 | .266 | .312 | .196 | .275 | .226 |
| 16 | 0 | 0 | 0 | 0 | .0307 | .106 | .131 | .27 | .112 | .267 | .297 | .256 | .218 | .239 | .151 | .337 | .246 |
| 17 | 0 | 0 | 0 | .0152 | .0451 | .132 | .16 | .25 | .241 | .291 | .282 | .246 | .23 | .179 | .202 | .325 | .233 |
| 18 | 0 | 0 | 0 | .0205 | .0729 | .147 | .135 | .163 | .129 | .202 | .187 | .208 | .19 | .192 | .211 | .166 | .168 |
| 19 | 0 | 0 | 0 | .0153 | .0799 | .124 | .159 | .197 | .198 | .227 | .209 | .23 | .221 | .313 | .241 | .28 | .314 |
| 20 | 0 | 0 | 0 | .043 | .118 | .154 | .141 | .199 | .213 | .241 | .279 | .22 | .254 | .229 | .246 | .248 | .485 |
| 21 | 0 | 0 | 0 | 0 | 0 | .0363 | .045 | .0596 | .0512 | .0994 | .0691 | .0918 | .0831 | .0709 | .0765 | .0958 | .0985 |
| 22 | 0 | 0 | 0 | .0521 | .0827 | .123 | .127 | .169 | .199 | .256 | .247 | .222 | .182 | .253 | .304 | .289 | .291 |
| 23 | 0 | 0 | 0 | .0591 | .0853 | .164 | .164 | .201 | .185 | .234 | .216 | .316 | .211 | .249 | .29 | .272 | .363 |
| 24 | 0 | 0 | .0159 | .124 | .16 | .142 | .161 | .185 | .154 | .206 | .143 | .351 | .181 | .174 | .234 | .155 | .307 |
| 25 | 0 | 0 | 0 | .0235 | .0641 | .125 | .13 | .211 | .204 | .234 | .18 | .252 | .233 | .252 | .24 | .228 | .269 |
| 26 | 0 | 0 | .0265 | .0823 | .115 | .159 | .171 | .165 | .239 | .215 | .199 | .303 | .249 | .379 | .212 | .203 | .217 |
| 27 | 0 | 0 | 0 | .0286 | .0618 | .0766 | .109 | .192 | .153 | .13 | .12 | .204 | .206 | .149 | .201 | .183 | .11 |
| 28 | 0 | 0 | 0 | .0276 | .0929 | .167 | .209 | .2 | .174 | .204 | .253 | .299 | .23 | .304 | .25 | .236 | .244 |
| 29 | 0 | 0 | 0 | .0439 | .0887 | 1.28 | .183 | .239 | .248 | .286 | .299 | .396 | .314 | .267 | .334 | .299 | .311 |
| 30 | 0 | 0 | 0 | 0 | .0169 | .0648 | .0876 | .114 | .151 | .132 | .0937 | .155 | .129 | .172 | .107 | .113 | .142 |

| subj. | time [h] | | | | | | | | | | | | | | | |
|---|---|---|---|---|---|---|---|---|---|---|---|---|---|---|---|---|
| no. | 288 | 312 | 316 | 320 | 324 | 336 | 337 | 338 | 339 | 340 | 342 | 344 | 350 | 360 | 372 | 384 |
| 01 | n.s. | n.s. | n.s. | n.s. | n.s. | n.s. | n.s. | n.s. | n.s. | n.s. | n.s. | n.s. | n.s. | n.s. | n.s. | n.s. |
| 02 | .307 | .268 | .291 | .385 | .357 | .298 | .284 | .218 | .207 | .16 | .111 | .0841 | .0327 | .02 | 0 | 0 |
| 03 | .205 | .246 | .175 | .192 | .196 | .169 | .17 | .135 | .148 | .124 | .0615 | .0474 | .0192 | .0109 | 0 | 0 |
| 04 | .15 | .274 | .152 | .233 | .164 | .13 | .134 | .118 | .094 | .0795 | .0514 | .0279 | .0164 | 0 | 0 | 0 |
| 05 | .363 | .407 | .355 | .311 | .335 | .262 | .184 | .146 | .139 | .0899 | .051 | .0303 | .017 | .0116 | 0 | 0 |
| 06 | .205 | .148 | .12 | .172 | .168 | .19 | .147 | .136 | .145 | .113 | .0761 | .0507 | .0299 | .0156 | 0 | 0 |
| 07 | .199 | .159 | .343 | .25 | .234 | .122 | .097 | .0773 | .06 | .0537 | .0212 | .0145 | 0 | 0 | 0 | 0 |
| 08 | .386 | .399 | .441 | .479 | .306 | .405 | .249 | .194 | .114 | .128 | .0873 | .0627 | .0303 | .0114 | 0 | .0129 |
| 09 | .292 | .398 | .254 | .342 | .356 | .334 | .233 | .177 | .158 | .139 | .0986 | .0387 | .0369 | .0159 | .0109 | 0 |
| 10 | .335 | .287 | .408 | .513 | .521 | .296 | .276 | .232 | .194 | .159 | .0893 | .0706 | .0402 | .034 | .0119 | .0112 |
| 11 | .097 | .0942 | .108 | .11 | .105 | .216 | .123 | .0932 | .084 | .0816 | .0595 | .0453 | .0268 | .0226 | 0 | .0114 |
| 12 | .365 | .293 | .657 | .814 | .933 | .156 | .12 | .0797 | .0847 | .0702 | .0535 | .0425 | .0235 | .02 | 0 | 0 |
| 13 | .233 | .289 | .148 | .182 | .159 | .183 | .161 | .111 | .108 | .0833 | .0647 | .0419 | .0222 | .0133 | 0 | 0 |
| 14 | .15 | .126 | .0971 | .124 | .144 | .0774 | .112 | .0809 | .0693 | .0452 | .0257 | .022 | .0114 | 0 | 0 | 0 |
| 15 | .227 | .274 | .244 | .244 | .272 | .229 | .228 | .189 | .187 | .13 | .0839 | .0599 | .0293 | .0137 | 0 | 0 |
| 16 | .276 | .304 | .177 | .232 | .227 | .266 | .197 | .146 | .134 | .124 | .107 | .0572 | .0386 | .016 | .0139 | 0 |
| 17 | .222 | .301 | .191 | .167 | .209 | .227 | .192 | .188 | .178 | .144 | .0967 | .0754 | .0442 | .0284 | .0124 | .0104 |
| 18 | .21 | .231 | .233 | .283 | .263 | .226 | .189 | .182 | .154 | .119 | .0851 | .0553 | .0204 | .0124 | 0 | 0 |
| 19 | .274 | .161 | .259 | .28 | .294 | .263 | .22 | .178 | .156 | .138 | .0952 | .0622 | .0348 | .0328 | 0 | 0 |
| 20 | .176 | .184 | .305 | .396 | .389 | .268 | .227 | .162 | .122 | .0984 | .068 | .0458 | .0158 | .0136 | 0 | 0 |
| 21 | .0996 | .0992 | .0675 | .0664 | .0756 | .0669 | .0801 | .0733 | .0647 | .0595 | .0392 | .0341 | .0123 | 0 | 0 | 0 |
| 22 | .191 | .183 | .232 | .216 | .277 | .239 | .16 | .136 | .109 | .0782 | .0539 | .0426 | .0198 | .0134 | 0 | 0 |
| 23 | .271 | .173 | .129 | .128 | .146 | .169 | .139 | .109 | .105 | .117 | .0618 | .0588 | .0312 | .0289 | .0127 | .0155 |
| 24 | .134 | .153 | .138 | .131 | .194 | .163 | .125 | .119 | .0806 | .0759 | .0487 | .0402 | .0102 | 0 | 0 | .0131 |
| 25 | .233 | .157 | .171 | .207 | .22 | .221 | .183 | .144 | .122 | .106 | .0683 | .0488 | .0219 | .0111 | 0 | 0 |
| 26 | .114 | .221 | .309 | .223 | .253 | .169 | .183 | .128 | .0972 | .107 | .0753 | .0569 | .0239 | .0544 | 0 | 0 |
| 27 | .216 | .146 | .176 | .206 | .206 | .296 | .118 | .125 | .121 | .106 | .0487 | .0351 | .018 | 0 | 0 | 0 |
| 28 | .247 | .266 | .373 | .394 | .495 | .233 | .203 | .201 | .192 | .137 | .105 | .0746 | .0416 | .0136 | .0103 | 0 |
| 29 | .287 | .36 | .385 | .46 | .403 | .447 | .283 | .207 | .201 | .168 | .119 | .0889 | .0452 | .0239 | .0143 | 0 |
| 30 | .158 | .157 | .0911 | .136 | .113 | .13 | .111 | .0887 | .0853 | .0708 | .0461 | .0309 | .0182 | .0118 | 0 | .0385 |

Dimension concentration [ng/ml]

TABLE 10
Parameters of model independent pharmacokinetics
of rotigotine during and after multiple transdermal
administration of 4.5 mg rotigotine with Patch C.

| subj. | Cₘₐₓ | tₘₐₓ | AUC(312-336) |
|---|---|---|---|
| 02 | .385 | 320.0 | 7.884 |
| 03 | .196 | 324.0 | 4.542 |
| 04 | .233 | 320.0 | 4.18 |
| 05 | .355 | 316.0 | 7.73 |
| 06 | .19 | 336.0 | 3.948 |
| 07 | .343 | 316.0 | 5.294 |
| 08 | .479 | 320.0 | 9.356 |
| 09 | .356 | 324.0 | 8.032 |
| 10 | .521 | 324.0 | 10.202 |
| 11 | .216 | 336.0 | 3.1964 |
| 12 | .933 | 324.0 | 14.87 |
| 13 | .183 | 336.0 | 4.268 |
| 14 | .144 | 324.0 | 2.7528 |
| 15 | .272 | 324.0 | 6.05 |
| 16 | .266 | 336.0 | 5.656 |
| 17 | .227 | 336.0 | 5.068 |
| 18 | .283 | 320.0 | 5.986 |
| 19 | .294 | 324.0 | 6.408 |
| 20 | .396 | 320.0 | 7.892 |
| 21 | .0756 | 324.0 | 1.7402 |
| 22 | .277 | 324.0 | 5.808 |
| 23 | .169 | 336.0 | 3.556 |
| 24 | .194 | 324.0 | 3.912 |
| 25 | .221 | 336.0 | 4.912 |
| 26 | .309 | 316.0 | 5.608 |
| 27 | .296 | 336.0 | 5.244 |
| 28 | .495 | 324.0 | 8.958 |
| 29 | .46 | 320.0 | 10.006 |
| 30 | .136 | 320.0 | 2.9064 |
| Min | .0756 | 316.0 | 1.7402 |
| Max | .933 | 336.0 | 14.87 |
| Med | .277 | 324.0 | 5.608 |
| \overline{x} | .307 | 325.517 | 6.068 |
| SD | .165 | 7.044 | 2.798 |

Dimension:
c-max [ng/ml];
t-max, t [h];
auc [ng/ml h]

### EXAMPLE 3

Study Design and Subject Population

A single-center, open-label, single-dose, randomized two-way cross-over clinical trial was performed to assess bioequivalence of two different rotigotine-containing silicone patches in 30 healthy male subjects (Caucasian, aged 18-50 years). The first silicone transdermal patches (Patch C) were made in accordance with the teachings of U.S. Patent Application Publication No. US 2003/0026830 at paragraphs 3842, U.S. Patent Application Publication No. US 2003/0027793 at paragraphs 3741 and U.S. Pat. No. 6,884,434, columns 5-6, Example 2 and comprised the following layers and components:

PATCH C

| Name of Ingredient | mg/10 cm² patch | |
|---|---|---|
| Rotigotine | 4.50 | |
| Silicone adhesive 4301 | 22.24 | |
| Silicone adhesive 4201 | 22.23 | |
| Providone | 1.00 | |
| Sodium metabisulfite | 0.00045 | |
| Ascorbyl palmitate | 0.010 | |
| Vitamin E (DL-α-tocopherol) | 0.025 | |
| Scotchpak 1109 (backing film) | 10 | cm² |

The second silicone transdermal patches (Patch D) were made in accordance with the teachings of U.S. Patent Application Publication No. US 2003/0026830 at paragraphs 3842 and U.S. Patent Application Publication No. US 2003/0027793 at paragraphs 3741 and comprised the following layers and components:

Patch D

| Name of Ingredient | mg/10 cm² patch | |
|---|---|---|
| Rotigotine | 4.50 | |
| Silicone adhesive 4301 | 22.24 | |
| Silicone adhesive 4201 | 22.23 | |
| Providone | 1.00 | |
| Sodium metabisulfite | 0.00045 | |
| Ascorbyl palmitate | 0.010 | |
| Vitamin E (DL-α-tocopherol) | 0.025 | |
| Backing foil PET, siliconized aluminized, color coated | 10 | cm² |

| | |
|---|---|
| Ink Bargofor 70135-1-P | As much |
| | as needed |

Both patch types contained 4.5 mg rotigotine/10 cm². In a first period, patches were administered singly to the subjects for 24 hours. After a washout period of 7 days, the other patch was administered for 24 hours.

During the study blood samples for the analysis of rotigotine were taken before patch application and at, 1, 2, 4, 6, 8, 10, 12, 15, 23, 24, 25, 26, 27, 28, 30, 36 and 48 hours after first patch application The study was done under in-patient conditions except for the urine collection at 36-48 hours and the 48 h blood collection (which were performed on an ambulatory basis).

Plasma Concentrations of Rotigotine

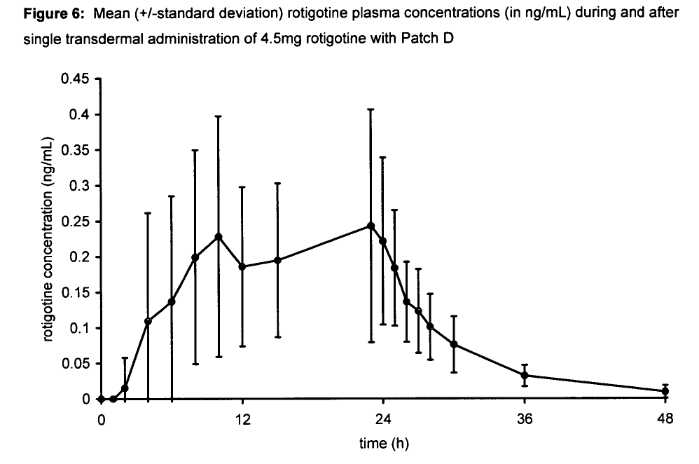

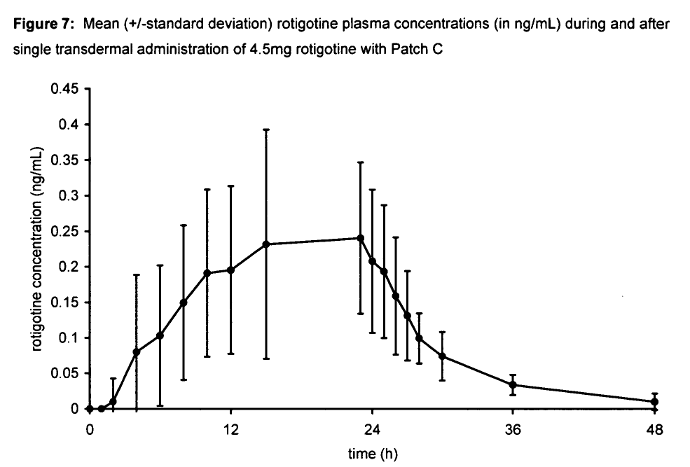

Data for rotigotine plasma concentrations and pharmacokinetic parameters measured during this clinical are provided in Tables 11, 12, 13, 14, and 15. FIGS. 6 and 7 illustrate the arithmetic mean of rotigotine plasma concentration for single patch administration. Table 15 summarizes the results of a statistical test to show that the two patch formulations are bioequivalent.

TABLE 11
Mean rotigotine plasma concentrations (in ng/mL) during
and after transdermal administration of 4.5 mg rotigotine with Patch D.

| Time [h] | Mean | std | Minimum | Maximum | Median | n |
|---|---|---|---|---|---|---|
| 0 | 0.000 | 0.000 | 0 | 0 | 0 | 30 |
| 1 | 0.000 | 0.000 | 0 | 0 | 0 | 30 |
| 2 | 0.015 | 0.043 | 0 | 177 | 0 | 30 |
| 4 | 0.109 | 0.152 | 0 | 622 | 66.4 | 30 |
| 6 | 0.137 | 0.148 | 0 | 761 | 106.8 | 30 |
| 8 | 0.199 | 0.150 | 15 | 685 | 170 | 30 |
| 10 | 0.228 | 0.169 | 38 | 762 | 185.5 | 30 |
| 12 | 0.186 | 0.112 | 69.2 | 525 | 157 | 30 |
| 15 | 0.194 | 0.108 | 48 | 505 | 164.5 | 30 |
| 23 | 0.243 | 0.163 | 73.8 | 766 | 190.5 | 30 |
| 24 | 0.221 | 0.117 | 95.9 | 556 | 197 | 28 |
| 25 | 0.184 | 0.081 | 56.1 | 378 | 159 | 30 |
| 26 | 0.136 | 0.056 | 42.3 | 264 | 132.5 | 28 |
| 27 | 0.123 | 0.059 | 39.3 | 259 | 113 | 29 |
| 28 | 0.101 | 0.046 | 23.1 | 207 | 94.35 | 30 |
| 30 | 0.076 | 0.040 | 31.3 | 200 | 62.85 | 30 |
| 36 | 0.032 | 0.015 | 10.3 | 61.4 | 30.65 | 30 |
| 48 | 0.009 | 0.009 | 0 | 28.3 | 11 | 30 |

TABLE 12
Mean rotigotine plasma concentrations (in ng/mL) during and after
transdermal administration of 4.5 mg rotigotine with Patch C

| Time [h] | Mean | std | Minimum | Maximum | Median | n |
|---|---|---|---|---|---|---|
| 0 | 0.000 | 0.000 | 0 | 0 | 0 | 30 |
| 1 | 0.000 | 0.000 | 0 | 0 | 0 | 30 |
| 2 | 0.010 | 0.032 | 0 | 159 | 0 | 30 |
| 4 | 0.080 | 0.109 | 0 | 448 | 34.55 | 30 |
| 6 | 0.103 | 0.099 | 0 | 457 | 65.75 | 30 |
| 8 | 0.150 | 0.109 | 20.1 | 453 | 121 | 30 |
| 10 | 0.191 | 0.117 | 23 | 520 | 151 | 30 |
| 12 | 0.195 | 0.118 | 35.2 | 511 | 150.5 | 30 |
| 15 | 0.232 | 0.161 | 74.9 | 737 | 182 | 30 |
| 23 | 0.240 | 0.106 | 98.1 | 589 | 243 | 29 |
| 24 | 0.208 | 0.101 | 60.2 | 505 | 186.5 | 30 |
| 25 | 0.193 | 0.093 | 48 | 508 | 177 | 30 |
| 26 | 0.159 | 0.082 | 66.2 | 371 | 149 | 29 |
| 27 | 0.131 | 0.063 | 65.1 | 307 | 120 | 29 |
| 28 | 0.099 | 0.035 | 29.2 | 179 | 100 | 30 |
| 30 | 0.074 | 0.034 | 26.6 | 156 | 70.6 | 29 |
| 36 | 0.034 | 0.014 | 0 | 59.2 | 32.5 | 30 |
| 48 | 0.010 | 0.012 | 0 | 33.6 | 5.2 | 30 |

TABLE 13
Parameters of model independent pharmacokinetics of rotigotine under
administration of 4.5 mg rotigotine with Patch D

| | | | | | Maxi- | % |
|---|---|---|---|---|---|---|
| Parameter | N | Mean | Std | Minimum | mum | CV |
| AUC | 27 | 5646.9 | 3031.1 | 2083.9 | 13379 | 53.7 |
| (0-tz) | | | | | | |
| AUC | 23 | 5736.1 | 2975.7 | 2251.9 | 13589 | 51.9 |
| (0-inf) | | | | | | |
| Cmax | 27 | 323.2 | 180.8 | 109 | 766 | 56.0 |
| Tmax | 27 | 17.1 | 6.8 | 4 | 27 | 39.5 |
| k | 23 | 0.1216 | 0.0383 | 0.0575 | 0.2083 | 31.5 |
| t1/2 | 23 | 6.2856 | 2.0848 | 3.3275 | 12.058 | 33.2 |

TABLE 14
Parameters of model independent pharmacokinetics of rotigotine
under administration of 4.5 mg rotigotine with Patch C

| | | | | | | % |
|---|---|---|---|---|---|---|
| Parameter | N | Mean | Std | Minimum | Maximum | CV |
| AUC(0- | 30 | 5307.2 | 2571.4 | 2134.3 | 12583 | 48.5 |
| tz) | | | | | | |
| AUC(0- | 23 | 5734.7 | 2553.8 | 2355.9 | 13070 | 44.5 |
| inf) | | | | | | |
| Cmax | 30 | 307.6 | 152.3 | 98.1 | 737 | 49.5 |
| Tmax | 30 | 17.5 | 6.5 | 4 | 26 | 37.3 |
| k | 23 | 0.1113 | 0.0500 | 0.0646 | 0.2598 | 44.9 |
| t½ | 23 | 7.1416 | 2.3062 | 2.6684 | 10.729 | 32.3 |

TABLE 15
Results of relative bioavailability for rotigoine after administration of Patches C and D

| | AUC₍₀₋ₜ₎ | AUC₍₀₋ₜ₎ | | | | |
|---|---|---|---|---|---|---|
| Parameter | (n = 27) | (n = 30) | AUC_{0-inf} | Cₘₐₓ | T_{1/2} | tₘₐₓ |
| Unit | h * ng/ml | h * ng/ml | h * ng/ml | ng/ml | h | h |
| Mean* test | 5.0117 | 4.7526 | 4.9491 | 0.2841 | 6.0598 | 15.0 |
| Mean* reference | 4.8262 | 4.7863 | 5.2203 | 0.2766 | 6.5673 | 15.0 |
| Difference | 0.0377 | −0.007 | −0.053 | 0.0267 | −0.08 | — |
| SE of Difference | 0.0567 | 0.0602 | 0.0722 | 0.0836 | 0.1093 | — |
| ratio | 103.85% | 99.30% | 94.81% | 102.70% | 92.27% | 96.67% |
| 90% CI lower limit | 94.26% | 89.63% | 83.52% | 89.04% | 76.18% | 83.33% |
| 90% CI upper limit | 114.39% | 110.00% | 107.60% | 118.46% | 111.76% | 113.34% |

*= least square means,
SE = standard error,
CI = confidence interval,
Difference = difference on log-scale

### EXAMPLE 4

Study Design and Subject Population

An open-label, multi-site, randomized trial with daily doses of rotigotine patch applied to the skin of 70 subjects was performed to evaluate the safety, tolerability, and effectiveness of placing the patch on different body sites. The study also evaluated electrocardiographic effects of patch-administered rotigotine. Each day, a fresh patch was placed on a new skin site (abdomen, flank, upper arm, shoulder, thigh, hip) in a rotating order. The silicone transdermal patches were made in accordance with the teachings of U.S. Patent Application Publication No. US 2003/0026830 at paragraphs 38-42, U.S. Patent Application Publication No. US 2003/0027793 at paragraphs 37-41 and U.S. Pat. No. 6,884,434, columns 5-6, Example 2 and comprised the following layers and components:

Patches D, E and F

| | Patch D (mg/10 | | Patch E (mg/20 | | Patch F (mg/30 | |
|---|---|---|---|---|---|---|
| Name of Ingredient | cm² patch) | | cm² patch) | | cm² patch) | |
| Rotigotine | 4.50 | | 9.00 | | 13.50 | |
| Silicone adhesive 4301 | 22.24 | | 44.47 | | 66.71 | |
| Silicone adhesive 4201 | 22.23 | | 44.46 | | 66.70 | |
| Providone | 1.00 | | 2.00 | | 3.00 | |
| Sodium metabisulfite | 0.00045 | | 0.0009 | | 0.00135 | |
| Ascorbyl palmitate | 0.010 | | 0.02 | | 0.03 | |
| Vitamin E | 0.025 | | 0.05 | | 0.075 | |
| (DL-α-tocopherol) | | | | | | |
| Backing foil PET, | 10 | cm² | 20 | cm² | 30 | cm² |
| siliconized aluminized, | | | | | | |
| color coated | | | | | | |

| | | | |
|---|---|---|---|
| rgofor | As much as | As much as | As much as |
| 70135-1-P | needed | needed | needed |

Rotigotine doses included 4.5 mg/day (Patch D), 9.0 mg/day (Patch E), 13.5 mg/day (Patch F), and 18.0 mg/day (2× Patch E). The trial consisted of an Eligibility Assessment (EA), a 24-day Titration Phase (4.5 to 18.0 mg/day doses; incremental increases of 4.5mg/day every 6 days), a 6-day Maintenance Phase (18.0 mg/day dose), a 6-day De-escalation Phase (13.5/9.0/4.5mg/day decreasing dose every 2 days), and a Safety Follow-Up visit 2 days following the last dose. A total of 70 subjects were enrolled and randomized; 63 subjects were analyzed for the primary pharmacokinetic (PK) variables and 58 subjects were analyzed for the primary pharmacodynamic variables.

The objectives of this trial included the following: 1) to characterize the pharmacokinetic profile of rotigotine during 24 hour intervals where the skin site of patch application was rotated in subjects with early-stage Parkinson's disease, 2) to investigate the electrocardiographic effects of rotigotine over a 24 hour period under maximal anticipated therapeutic exposure in subjects with early-stage Parkinson's disease, and 3) to investigate the safety and local tolerability of a rotigotine transdermal patch under maximal anticipated therapeutic exposure.

The study used 10 cm², 20 cm², and 30 cm² rotigotine transdermal patches, which correspond to 4.5 mg, 9.0 mg, and 13.5 mg rotigotine, respectively. The silicone transdermal patches were made in accordance with the teachings of U.S. Patent Application Publication No. US 2003/0026830 at paragraphs 3842 and U.S. Patent Application Publication No. US 2003/0027793 at paragraphs 3741 comprised the following layers and components as disclosed above

The 18.0 mg/day dose used 2×20 cm² patches. Initial doses were 4.5 mg/day with weekly increases of 4.5 mg/day to a maximum target dose of 18.0 mg/day.

Blood samples were collected before patch administration and on the days and at the times indicated in Table 16.

Plasma Concentrations of Rotigotine

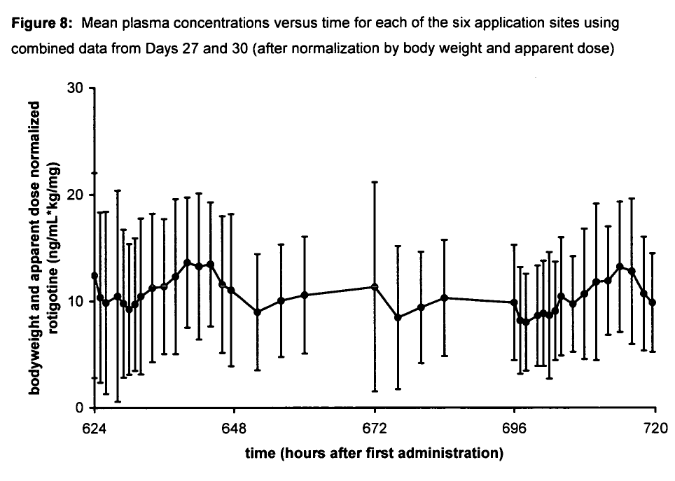

Mean plasma concentrations versus time for each of the six application sites using combined data from Days 27 and 30 are shown in the FIG. 8.

Mean plasma concentrations of unconjugated rotigotine were similar between the six application sites. Starting with a plasma concentration at Time 0 (prior to patch removal, C_{trough}) of about 1 ng/mL, the concentration decreased within 2 hours by about 0.2 ng/mL, followed by an increase back up to the level of the trough plasma concentration. FIG. 9 illustrates a plasma concentration over time for all patch application sites.

Table 16 reports the result of descriptive statistics of plasma concentrations for unconjugated rotigotine separated by the day of administration, the time of sampling after actual administration and the site of patch administration.

TABLE 16
Descriptive statistics of parameters of rotigotine plasma concentrations (ng/mL) under
multiple dose in patients with early-stage Parkinson's disease

| | | | | | | | | | | | |
|---|---|---|---|---|---|---|---|---|---|---|---|
| | | | # Obs. | | | | | | | | |
| Day | Time | n | >LOQ | Mean | SD | CV (%) | Geo. Mean | Geo. SD | Median | Min | Max |

Application Site = Hip

| | | | | | | | | | | | |
|---|---|---|---|---|---|---|---|---|---|---|---|
| Day 25 | 0 H | 10 | 10 | 0.9598 | 0.89990 | 93.8 | 0.7010 | 2.20222 | 0.5265 | 0.271 | 2.980 |
| | 4 H | 10 | 10 | 0.8993 | 0.67741 | 75.3 | 0.6929 | 2.16680 | 0.8040 | 0.256 | 2.400 |
| | 8 H | 10 | 10 | 0.8447 | 0.78229 | 92.6 | 0.5629 | 2.78343 | 0.6550 | 0.114 | 2.750 |
| | 12 H | 10 | 10 | 0.9776 | 0.79679 | 81.5 | 0.6434 | 2.94450 | 0.7255 | 0.094 | 2.330 |
| Day 26 | 0 H | 11 | 11 | 0.7716 | 0.39609 | 51.3 | 0.6542 | 1.93335 | 0.7250 | 0.187 | 1.260 |
| | 4 H | 11 | 11 | 0.5491 | 0.27813 | 50.7 | 0.4660 | 1.94460 | 0.5250 | 0.111 | 0.912 |
| | 8 H | 11 | 11 | 0.8153 | 0.44074 | 54.1 | 0.6802 | 1.98291 | 0.7620 | 0.230 | 1.370 |
| | 12 H | 11 | 11 | 0.9108 | 0.62860 | 69.0 | 0.6841 | 2.43929 | 0.9420 | 0.102 | 2.300 |
| Day 27 | 0 H | 10 | 10 | 1.3040 | 1.11784 | 85.7 | 1.0111 | 2.05974 | 0.8805 | 0.375 | 4.150 |
| | 1 H | 10 | 10 | 1.2353 | 1.05886 | 85.7 | 0.9845 | 1.92688 | 0.9300 | 0.450 | 3.980 |
| | 2 H | 10 | 10 | 0.9798 | 0.95288 | 97.3 | 0.7477 | 2.03439 | 0.7055 | 0.292 | 3.550 |
| | 4 H | 10 | 10 | 1.0117 | 1.26502 | 125.0 | 0.6774 | 2.27514 | 0.5055 | 0.314 | 4.440 |
| | 5 H | 10 | 10 | 0.8224 | 0.62891 | 76.5 | 0.6431 | 2.09471 | 0.6425 | 0.232 | 1.970 |
| | 6 H | 10 | 10 | 0.7661 | 0.45889 | 59.9 | 0.6546 | 1.80767 | 0.5935 | 0.283 | 1.560 |
| | 7 H | 10 | 10 | 0.8542 | 0.45527 | 53.3 | 0.7356 | 1.85188 | 0.7095 | 0.203 | 1.730 |
| | 8 H | 10 | 10 | 0.8676 | 0.43667 | 50.3 | 0.7580 | 1.78669 | 0.7310 | 0.236 | 1.520 |
| | 10 H | 10 | 10 | 0.9860 | 0.47824 | 48.5 | 0.8971 | 1.57901 | 0.8460 | 0.371 | 2.130 |
| | 12 H | 10 | 10 | 1.0611 | 0.55365 | 52.2 | 0.9429 | 1.67691 | 0.8825 | 0.380 | 2.280 |
| | 14 H | 10 | 10 | 1.0745 | 0.55728 | 51.9 | 0.9609 | 1.63250 | 0.9115 | 0.485 | 2.080 |
| | 16 H | 10 | 10 | 1.3677 | 0.79763 | 58.3 | 1.2073 | 1.65233 | 1.0500 | 0.634 | 3.090 |
| | 18 H | 10 | 10 | 1.1769 | 0.54621 | 46.4 | 1.0728 | 1.57672 | 0.9900 | 0.457 | 2.140 |
| | 20 H | 10 | 10 | 1.2121 | 0.51482 | 42.5 | 1.1143 | 1.54847 | 1.0650 | 0.593 | 1.970 |
| | 22 H | 10 | 10 | 0.9248 | 0.45066 | 48.7 | 0.8350 | 1.61184 | 0.8890 | 0.379 | 1.920 |
| | 23.5 H | 10 | 10 | 0.9600 | 0.38736 | 40.4 | 0.8745 | 1.64926 | 0.9570 | 0.261 | 1.750 |
| Day 28 | 4 H | 11 | 11 | 0.7955 | 0.46452 | 58.4 | 0.6786 | 1.82902 | 0.5510 | 0.218 | 1.570 |
| | 8 H | 11 | 11 | 1.1237 | 0.75207 | 66.9 | 0.8577 | 2.34277 | 1.0500 | 0.178 | 2.300 |
| | 12 H | 11 | 11 | 1.0759 | 0.65385 | 60.8 | 0.8227 | 2.44476 | 1.0000 | 0.158 | 2.130 |
| Day 29 | 0 H | 11 | 11 | 0.6160 | 0.28338 | 46.0 | 0.5494 | 1.69457 | 0.6300 | 0.209 | 1.070 |
| | 4 H | 11 | 11 | 0.4802 | 0.28770 | 59.9 | 0.4018 | 1.91554 | 0.3910 | 0.131 | 0.974 |
| | 8 H | 11 | 11 | 0.5221 | 0.26648 | 51.0 | 0.4468 | 1.88281 | 0.6020 | 0.133 | 0.900 |
| | 12 H | 11 | 11 | 0.5715 | 0.29201 | 51.1 | 0.5056 | 1.70189 | 0.4600 | 0.180 | 1.140 |
| Day 30 | 0 H | 10 | 10 | 1.0048 | 0.46928 | 46.7 | 0.9083 | 1.61927 | 0.8680 | 0.398 | 1.930 |
| | 1 H | 10 | 10 | 0.8518 | 0.36378 | 42.7 | 0.7819 | 1.56060 | 0.7840 | 0.393 | 1.500 |
| | 2 H | 10 | 10 | 0.6981 | 0.24795 | 35.5 | 0.6594 | 1.42980 | 0.6430 | 0.397 | 1.100 |
| | 4 H | 10 | 10 | 0.7967 | 0.68299 | 85.7 | 0.6487 | 1.85828 | 0.5950 | 0.236 | 2.670 |
| | 5 H | 10 | 10 | 0.8458 | 0.63315 | 74.9 | 0.6787 | 2.01882 | 0.7460 | 0.194 | 2.420 |
| | 6 H | 10 | 10 | 1.0816 | 1.52030 | 140.6 | 0.6542 | 2.61354 | 0.5810 | 0.140 | 5.310 |
| | 7 H | 10 | 10 | 0.7566 | 0.47940 | 63.4 | 0.6270 | 1.94917 | 0.6825 | 0.217 | 1.800 |
| | 8 H | 10 | 10 | 0.9441 | 0.99194 | 105.1 | 0.6780 | 2.25518 | 0.7225 | 0.214 | 3.640 |
| | 10 H | 10 | 10 | 0.7786 | 0.56988 | 73.2 | 0.6330 | 1.96963 | 0.6990 | 0.235 | 2.220 |
| | 12 H | 10 | 10 | 0.9966 | 0.64950 | 65.2 | 0.8291 | 1.91705 | 0.8425 | 0.317 | 2.510 |
| | 14 H | 10 | 10 | 0.8877 | 0.48873 | 55.1 | 0.7835 | 1.67757 | 0.7380 | 0.428 | 1.860 |
| | 16 H | 10 | 10 | 1.2098 | 0.66582 | 55.0 | 1.0564 | 1.74629 | 1.2200 | 0.424 | 2.670 |
| | 18 H | 10 | 10 | 1.2131 | 0.63776 | 52.6 | 1.0663 | 1.71951 | 1.0250 | 0.473 | 2.220 |
| | 20 H | 10 | 10 | 1.2665 | 0.70594 | 55.7 | 1.1060 | 1.72835 | 1.0255 | 0.504 | 2.600 |
| | 22 H | 10 | 10 | 1.0856 | 0.71368 | 65.7 | 0.9142 | 1.84220 | 0.9330 | 0.380 | 2.770 |
| | 23.5 H | 10 | 10 | 0.8204 | 0.34090 | 41.6 | 0.7585 | 1.51856 | 0.6915 | 0.461 | 1.350 |
| Days | 0 H | 20 | 20 | 1.1544 | 0.84839 | 73.5 | 0.9583 | 1.82270 | 0.8730 | 0.375 | 4.150 |
| 27 & 30 | 1 H | 20 | 20 | 1.0436 | 0.79528 | 76.2 | 0.8774 | 1.74752 | 0.8405 | 0.393 | 3.980 |
| Combined | 2 H | 20 | 20 | 0.8390 | 0.69289 | 82.6 | 0.7021 | 1.73502 | 0.6575 | 0.292 | 3.550 |
| | 4 H | 20 | 20 | 0.9042 | 0.99557 | 110.1 | 0.6629 | 2.03165 | 0.5520 | 0.236 | 4.440 |
| | 5 H | 20 | 20 | 0.8341 | 0.61432 | 73.7 | 0.6607 | 2.01882 | 0.6425 | 0.194 | 2.420 |
| | 6 H | 20 | 20 | 0.9239 | 1.10489 | 119.6 | 0.6544 | 2.17423 | 0.5905 | 0.140 | 5.310 |
| | 7 H | 20 | 20 | 0.8054 | 0.45777 | 56.8 | 0.6791 | 1.87859 | 0.6980 | 0.203 | 1.800 |
| | 8 H | 20 | 20 | 0.9059 | 0.74695 | 82.5 | 0.7169 | 1.99371 | 0.7225 | 0.214 | 3.640 |
| | 10 H | 20 | 20 | 0.8823 | 0.52297 | 59.3 | 0.7536 | 1.80455 | 0.7605 | 0.235 | 2.220 |
| | 12 H | 20 | 20 | 1.0289 | 0.58831 | 57.2 | 0.8842 | 1.77857 | 0.8675 | 0.317 | 2.510 |
| | 14 H | 20 | 20 | 0.9811 | 0.51907 | 52.9 | 0.8677 | 1.65125 | 0.8370 | 0.428 | 2.080 |
| | 16 H | 20 | 20 | 1.2888 | 0.71967 | 55.8 | 1.1294 | 1.68360 | 1.1500 | 0.424 | 3.090 |
| | 18 H | 20 | 20 | 1.1950 | 0.57821 | 48.4 | 1.0695 | 1.62781 | 1.0250 | 0.457 | 2.220 |
| | 20 H | 20 | 20 | 1.2393 | 0.60198 | 48.6 | 1.1101 | 1.61944 | 1.0650 | 0.504 | 2.600 |
| | 22 H | 20 | 20 | 1.0052 | 0.58675 | 58.4 | 0.8737 | 1.70855 | 0.9280 | 0.379 | 2.770 |
| | 23.5 H | 20 | 20 | 0.8902 | 0.36229 | 40.7 | 0.8144 | 1.57540 | 0.8700 | 0.261 | 1.750 |

Application Site = Shoulder

| | | | | | | | | | | | |
|---|---|---|---|---|---|---|---|---|---|---|---|
| Day 25 | 0 H | 11 | 11 | 0.8450 | 0.39342 | 46.6 | 0.7595 | 1.66491 | 0.7700 | 0.243 | 1.720 |
| | 4 H | 11 | 11 | 1.4656 | 1.18532 | 80.9 | 1.0877 | 2.29840 | 1.1300 | 0.276 | 4.130 |
| | 8 H | 11 | 11 | 1.5562 | 0.89190 | 57.3 | 1.2839 | 2.01651 | 1.2700 | 0.390 | 2.750 |
| | 12 H | 11 | 11 | 1.4674 | 0.87603 | 59.7 | 1.2450 | 1.84120 | 1.4900 | 0.534 | 3.390 |
| Day 26 | 0 H | 11 | 11 | 0.9283 | 0.80108 | 86.3 | 0.7068 | 2.15694 | 0.7510 | 0.170 | 3.020 |
| | 4 H | 11 | 11 | 0.8209 | 0.43510 | 53.0 | 0.7038 | 1.85972 | 0.9000 | 0.212 | 1.600 |
| | 8 H | 11 | 11 | 1.0675 | 0.51786 | 48.5 | 0.9399 | 1.74736 | 1.1800 | 0.338 | 2.020 |
| | 12 H | 11 | 11 | 1.1026 | 0.41510 | 37.6 | 1.0094 | 1.61600 | 1.2600 | 0.373 | 1.700 |
| Day 27 | 0 H | 11 | 11 | 0.7905 | 0.58843 | 74.4 | 0.6131 | 2.13333 | 0.5090 | 0.188 | 1.880 |
| | 1 H | 11 | 11 | 0.7317 | 0.46938 | 64.1 | 0.6025 | 1.93539 | 0.6100 | 0.239 | 1.470 |
| | 2 H | 11 | 11 | 0.7235 | 0.38012 | 52.5 | 0.6190 | 1.86084 | 0.6960 | 0.223 | 1.360 |
| | 4 H | 11 | 11 | 0.7431 | 0.44194 | 59.5 | 0.6375 | 1.78507 | 0.6880 | 0.283 | 1.700 |
| | 5 H | 11 | 11 | 0.7521 | 0.56646 | 75.3 | 0.6188 | 1.86218 | 0.5000 | 0.293 | 2.250 |
| | 6 H | 11 | 11 | 0.7916 | 0.63449 | 80.1 | 0.6556 | 1.80693 | 0.6020 | 0.287 | 2.580 |
| | 7 H | 11 | 11 | 0.7798 | 0.44581 | 57.2 | 0.6828 | 1.71130 | 0.6450 | 0.258 | 1.830 |
| | 8 H | 11 | 11 | 0.8102 | 0.59484 | 73.4 | 0.6695 | 1.86679 | 0.5890 | 0.253 | 2.370 |
| | 10 H | 11 | 11 | 0.8745 | 0.42362 | 48.4 | 0.7815 | 1.67369 | 0.8150 | 0.290 | 1.790 |
| | 12 H | 11 | 11 | 0.8646 | 0.43447 | 50.2 | 0.7759 | 1.63179 | 0.7820 | 0.339 | 1.810 |
| | 14 H | 10 | 10 | 0.9155 | 0.37149 | 40.6 | 0.8388 | 1.58945 | 0.9210 | 0.357 | 1.530 |
| | 16 H | 11 | 11 | 1.0633 | 0.57402 | 54.0 | 0.9311 | 1.72241 | 0.8600 | 0.412 | 2.250 |
| | 18 H | 11 | 11 | 1.1540 | 0.58214 | 50.4 | 1.0212 | 1.69632 | 1.0400 | 0.431 | 2.230 |
| | 20 H | 11 | 11 | 1.0924 | 0.45924 | 42.0 | 0.9953 | 1.61217 | 1.0400 | 0.372 | 2.000 |
| | 22 H | 11 | 11 | 0.9357 | 0.41007 | 43.8 | 0.8398 | 1.68826 | 0.9560 | 0.289 | 1.670 |
| | 23.5 H | 11 | 11 | 0.9075 | 0.52690 | 58.1 | 0.7901 | 1.71665 | 0.7130 | 0.387 | 1.980 |
| Day 28 | 4 H | 9 | 9 | 1.0613 | 0.84706 | 79.8 | 0.8260 | 2.08735 | 0.6080 | 0.323 | 2.940 |
| | 8 H | 9 | 9 | 1.1016 | 1.07009 | 97.1 | 0.7720 | 2.46350 | 0.8690 | 0.199 | 3.710 |
| | 12 H | 9 | 9 | 1.0689 | 0.84181 | 78.8 | 0.8423 | 2.10124 | 0.9110 | 0.214 | 3.100 |
| Day 29 | 0 H | 10 | 10 | 0.8564 | 0.37266 | 43.5 | 0.7862 | 1.55758 | 0.8040 | 0.347 | 1.650 |
| | 4 H | 10 | 10 | 1.0285 | 0.50781 | 49.4 | 0.8838 | 1.89291 | 0.9695 | 0.238 | 1.640 |
| | 8 H | 10 | 10 | 1.1192 | 0.82177 | 73.4 | 0.8413 | 2.34757 | 0.8730 | 0.163 | 2.560 |
| | 12 H | 10 | 10 | 1.0289 | 0.81504 | 79.2 | 0.7421 | 2.45889 | 0.7160 | 0.163 | 2.620 |
| Day 30 | 0 H | 11 | 11 | 0.6888 | 0.32132 | 46.6 | 0.6271 | 1.57060 | 0.6710 | 0.316 | 1.330 |
| | 1 H | 11 | 11 | 0.4812 | 0.17361 | 36.1 | 0.4525 | 1.45073 | 0.4390 | 0.239 | 0.745 |
| | 2 H | 11 | 11 | 0.6484 | 0.31245 | 48.2 | 0.5870 | 1.59214 | 0.6530 | 0.334 | 1.360 |
| | 4 H | 11 | 11 | 1.0701 | 0.65380 | 61.1 | 0.9207 | 1.77194 | 0.9420 | 0.380 | 2.720 |
| | 5 H | 11 | 11 | 1.1798 | 0.86047 | 72.9 | 0.9713 | 1.88398 | 0.9560 | 0.360 | 3.330 |
| | 6 H | 11 | 11 | 0.9113 | 0.38029 | 41.7 | 0.8369 | 1.56205 | 0.8190 | 0.364 | 1.530 |
| | 7 H | 11 | 11 | 1.0807 | 0.45844 | 42.4 | 0.9932 | 1.55158 | 1.0800 | 0.438 | 1.870 |
| | 8 H | 11 | 11 | 1.2537 | 0.84729 | 67.6 | 1.0561 | 1.82857 | 1.0200 | 0.372 | 3.410 |
| | 10 H | 11 | 11 | 1.1660 | 0.55622 | 47.7 | 1.0307 | 1.73365 | 1.0800 | 0.369 | 1.980 |
| | 12 H | 11 | 11 | 1.1693 | 0.54122 | 46.3 | 1.0404 | 1.70227 | 1.0500 | 0.401 | 1.890 |
| | 14 H | 11 | 11 | 1.2580 | 0.58500 | 46.5 | 1.1460 | 1.57295 | 1.2100 | 0.580 | 2.590 |
| | 16 H | 11 | 11 | 1.2787 | 0.66304 | 51.9 | 1.1479 | 1.60896 | 1.0600 | 0.600 | 2.530 |
| | 18 H | 11 | 11 | 1.3215 | 0.77932 | 59.0 | 1.1604 | 1.67705 | 1.1700 | 0.577 | 3.250 |
| | 20 H | 11 | 11 | 1.3956 | 1.19600 | 85.7 | 1.1356 | 1.84877 | 1.0400 | 0.427 | 4.810 |
| | 22 H | 10 | 10 | 0.9039 | 0.43904 | 48.6 | 0.8153 | 1.61217 | 0.7335 | 0.398 | 1.770 |
| | 23.5 H | 11 | 11 | 1.0242 | 0.75976 | 74.2 | 0.8511 | 1.82764 | 0.8480 | 0.405 | 3.010 |
| Days | 0 H | 22 | 22 | 0.7397 | 0.46557 | 62.9 | 0.6201 | 1.83811 | 0.5915 | 0.188 | 1.880 |
| 27 & 30 | 1 H | 22 | 22 | 0.6065 | 0.36838 | 60.7 | 0.5222 | 1.72140 | 0.4560 | 0.239 | 1.470 |
| Combined | 2 H | 22 | 22 | 0.6859 | 0.34171 | 49.8 | 0.6028 | 1.70931 | 0.6910 | 0.223 | 1.360 |
| | 4 H | 22 | 22 | 0.9066 | 0.56971 | 62.8 | 0.7661 | 1.80861 | 0.7855 | 0.283 | 2.720 |
| | 5 H | 22 | 22 | 0.9660 | 0.74384 | 77.0 | 0.7753 | 1.92416 | 0.8045 | 0.293 | 3.330 |
| | 6 H | 22 | 22 | 0.8515 | 0.51412 | 60.4 | 0.7407 | 1.69270 | 0.7555 | 0.287 | 2.580 |
| | 7 H | 22 | 22 | 0.9303 | 0.46737 | 50.2 | 0.8235 | 1.67509 | 0.7655 | 0.258 | 1.870 |
| | 8 H | 22 | 22 | 1.0320 | 0.74958 | 72.6 | 0.8409 | 1.90210 | 0.9045 | 0.253 | 3.410 |
| | 10 H | 22 | 22 | 1.0203 | 0.50500 | 49.5 | 0.8975 | 1.71434 | 0.9450 | 0.290 | 1.980 |
| | 12 H | 22 | 22 | 1.0170 | 0.50366 | 49.5 | 0.8985 | 1.68374 | 0.8870 | 0.339 | 1.890 |
| | 14 H | 21 | 21 | 1.0949 | 0.51375 | 46.9 | 0.9878 | 1.60647 | 1.0700 | 0.357 | 2.590 |
| | 16 H | 22 | 22 | 1.1710 | 0.61515 | 52.5 | 1.0338 | 1.66506 | 1.0170 | 0.412 | 2.530 |
| | 18 H | 22 | 22 | 1.2378 | 0.67671 | 54.7 | 1.0886 | 1.67256 | 1.1350 | 0.431 | 3.250 |
| | 20 H | 22 | 22 | 1.2440 | 0.89759 | 72.2 | 1.0632 | 1.71821 | 1.0400 | 0.372 | 4.810 |
| | 22 H | 21 | 21 | 0.9206 | 0.41362 | 44.9 | 0.8280 | 1.63214 | 0.8480 | 0.289 | 1.770 |
| | 23.5 H | 22 | 22 | 0.9659 | 0.64081 | 66.3 | 0.8201 | 1.75077 | 0.7995 | 0.387 | 3.010 |

Application Site = Upper Arm

| | | | | | | | | | | | |
|---|---|---|---|---|---|---|---|---|---|---|---|
| Day 25 | 0 H | 10 | 10 | 0.8531 | 0.49953 | 58.6 | 0.7157 | 1.93040 | 0.7455 | 0.219 | 1.770 |
| | 4 H | 10 | 10 | 0.6055 | 0.25103 | 41.5 | 0.5603 | 1.51365 | 0.5080 | 0.366 | 1.000 |
| | 8 H | 10 | 10 | 0.7649 | 0.36745 | 48.0 | 0.6892 | 1.61891 | 0.6225 | 0.354 | 1.420 |
| | 12 H | 10 | 10 | 0.9058 | 0.38785 | 42.8 | 0.8191 | 1.65583 | 0.8865 | 0.283 | 1.540 |
| Day 26 | 0 H | 9 | 9 | 0.6452 | 0.26690 | 41.4 | 0.5823 | 1.69184 | 0.6370 | 0.192 | 1.080 |
| | 4 H | 9 | 9 | 0.9841 | 1.16480 | 118.4 | 0.6514 | 2.38356 | 0.4370 | 0.290 | 3.860 |
| | 8 H | 9 | 9 | 0.8018 | 0.58759 | 73.3 | 0.6614 | 1.88172 | 0.6230 | 0.292 | 2.170 |
| | 12 H | 9 | 9 | 0.9626 | 0.60654 | 63.0 | 0.8223 | 1.78312 | 0.6170 | 0.413 | 2.170 |
| Day 27 | 0 H | 10 | 10 | 1.1664 | 0.63917 | 54.8 | 0.8981 | 2.62949 | 1.0750 | 0.076 | 2.060 |
| | 1 H | 10 | 10 | 0.7850 | 0.39998 | 51.0 | 0.6288 | 2.42290 | 0.7060 | 0.062 | 1.400 |
| | 2 H | 10 | 10 | 0.7760 | 0.36374 | 46.9 | 0.6493 | 2.16745 | 0.6835 | 0.086 | 1.360 |
| | 4 H | 10 | 10 | 0.8503 | 0.46010 | 54.1 | 0.7529 | 1.67989 | 0.8085 | 0.358 | 1.920 |
| | 5 H | 10 | 10 | 1.0106 | 0.65776 | 65.1 | 0.8303 | 1.95398 | 0.8270 | 0.348 | 2.120 |
| | 6 H | 10 | 10 | 0.9577 | 0.62292 | 65.0 | 0.6714 | 3.17479 | 0.9535 | 0.036 | 2.160 |
| | 7 H | 10 | 10 | 0.8950 | 0.53831 | 60.1 | 0.6804 | 2.57511 | 0.9335 | 0.071 | 1.840 |
| | 8 H | 10 | 10 | 0.9987 | 0.55599 | 55.7 | 0.8537 | 1.84479 | 0.8595 | 0.383 | 2.030 |
| | 10 H | 10 | 10 | 0.9798 | 0.51868 | 52.9 | 0.8616 | 1.71881 | 0.8135 | 0.365 | 1.930 |
| | 12 H | 10 | 10 | 1.1466 | 0.63722 | 55.6 | 0.9701 | 1.92584 | 1.0255 | 0.253 | 2.290 |
| | 14 H | 10 | 10 | 1.0699 | 0.58804 | 55.0 | 0.8976 | 1.97808 | 0.9335 | 0.213 | 2.020 |
| | 16 H | 10 | 10 | 1.1540 | 0.58531 | 50.7 | 0.9705 | 2.03045 | 1.1030 | 0.187 | 1.910 |
| | 18 H | 10 | 10 | 1.1039 | 0.60875 | 55.1 | 0.9215 | 2.02065 | 0.9330 | 0.194 | 2.030 |
| | 20 H | 10 | 10 | 1.1564 | 0.75476 | 65.3 | 0.9511 | 1.99892 | 0.9700 | 0.228 | 2.870 |
| | 22 H | 10 | 10 | 1.0020 | 0.62301 | 62.2 | 0.8046 | 2.13992 | 0.8670 | 0.190 | 2.160 |
| | 23.5 H | 10 | 10 | 0.9361 | 0.55459 | 59.2 | 0.7423 | 2.26567 | 0.9385 | 0.129 | 1.880 |
| Day 28 | 4 H | 12 | 12 | 0.7681 | 0.51159 | 66.6 | 0.6211 | 1.99127 | 0.5480 | 0.251 | 1.720 |
| | 8 H | 12 | 12 | 1.0700 | 0.66177 | 61.8 | 0.8703 | 2.08162 | 1.0000 | 0.178 | 2.650 |
| | 12 H | 12 | 12 | 0.9082 | 0.46115 | 50.8 | 0.7892 | 1.79669 | 0.9415 | 0.249 | 1.740 |
| Day 29 | 0 H | 11 | 11 | 1.0746 | 0.72989 | 67.9 | 0.8627 | 2.10510 | 0.9570 | 0.180 | 2.870 |
| | 4 H | 11 | 11 | 0.8639 | 0.70262 | 81.3 | 0.6623 | 2.15445 | 0.6130 | 0.192 | 2.650 |
| | 8 H | 11 | 11 | 1.1498 | 0.67779 | 58.9 | 0.9496 | 1.99607 | 1.1600 | 0.306 | 2.470 |
| | 12 H | 11 | 11 | 1.2128 | 0.71743 | 59.2 | 1.0161 | 1.91241 | 1.1000 | 0.377 | 2.490 |
| Day 30 | 0 H | 11 | 11 | 0.7028 | 0.23782 | 33.8 | 0.6599 | 1.47979 | 0.6920 | 0.314 | 0.981 |
| | 1 H | 11 | 11 | 0.6353 | 0.31713 | 49.9 | 0.5665 | 1.65855 | 0.5710 | 0.269 | 1.230 |
| | 2 H | 11 | 11 | 0.5860 | 0.16988 | 29.0 | 0.5653 | 1.32079 | 0.5710 | 0.375 | 0.945 |
| | 4 H | 11 | 11 | 0.6831 | 0.30322 | 44.4 | 0.6218 | 1.59139 | 0.6410 | 0.278 | 1.260 |
| | 5 H | 11 | 11 | 0.7822 | 0.38194 | 48.8 | 0.6938 | 1.69794 | 0.8190 | 0.314 | 1.440 |
| | 6 H | 11 | 11 | 0.8920 | 0.45884 | 51.4 | 0.7891 | 1.69564 | 0.9590 | 0.381 | 1.910 |
| | 7 H | 11 | 11 | 0.7893 | 0.37701 | 47.8 | 0.7130 | 1.61065 | 0.8100 | 0.358 | 1.650 |
| | 8 H | 11 | 11 | 0.9771 | 0.39344 | 40.3 | 0.9046 | 1.52119 | 0.9690 | 0.449 | 1.770 |
| | 10 H | 11 | 11 | 1.0650 | 0.63744 | 59.9 | 0.9417 | 1.63598 | 0.9940 | 0.562 | 2.760 |
| | 12 H | 11 | 11 | 1.1607 | 0.69159 | 59.6 | 1.0052 | 1.74277 | 1.0600 | 0.475 | 2.830 |
| | 14 H | 11 | 11 | 1.0294 | 0.37927 | 36.8 | 0.9606 | 1.49647 | 1.0400 | 0.488 | 1.590 |
| | 16 H | 11 | 11 | 0.9613 | 0.45864 | 47.7 | 0.8572 | 1.68573 | 0.9020 | 0.312 | 1.720 |
| | 18 H | 11 | 11 | 1.1474 | 0.46970 | 40.9 | 1.0444 | 1.62876 | 1.2400 | 0.351 | 1.990 |
| | 20 H | 11 | 11 | 1.1207 | 0.59545 | 53.1 | 0.9702 | 1.80101 | 1.1300 | 0.354 | 2.230 |
| | 22 H | 11 | 11 | 1.0576 | 0.41511 | 39.2 | 0.9806 | 1.52585 | 0.9800 | 0.415 | 1.850 |
| | 23.5 H | 11 | 11 | 0.9245 | 0.41698 | 45.1 | 0.8333 | 1.64525 | 0.8990 | 0.347 | 1.670 |
| Days | 0 H | 21 | 21 | 0.9236 | 0.51808 | 56.1 | 0.7642 | 2.05994 | 0.8560 | 0.076 | 2.060 |
| 27 & 30 | 1 H | 21 | 21 | 0.7066 | 0.35798 | 50.7 | 0.5954 | 2.00405 | 0.6330 | 0.062 | 1.400 |
| Combined | 2 H | 21 | 21 | 0.6765 | 0.28882 | 42.7 | 0.6038 | 1.74974 | 0.6510 | 0.086 | 1.360 |
| | 4 H | 21 | 21 | 0.7627 | 0.38543 | 50.5 | 0.6811 | 1.62982 | 0.7560 | 0.278 | 1.920 |
| | 5 H | 21 | 21 | 0.8910 | 0.53037 | 59.5 | 0.7557 | 1.80767 | 0.8190 | 0.314 | 2.120 |
| | 6 H | 21 | 21 | 0.9233 | 0.53011 | 57.4 | 0.7307 | 2.37307 | 0.9590 | 0.036 | 2.160 |
| | 7 H | 21 | 21 | 0.8396 | 0.45210 | 53.8 | 0.6973 | 2.05213 | 0.8830 | 0.071 | 1.840 |
| | 8 H | 21 | 21 | 0.9874 | 0.46543 | 47.1 | 0.8800 | 1.66122 | 0.9540 | 0.383 | 2.030 |
| | 10 H | 21 | 21 | 1.0244 | 0.57108 | 55.7 | 0.9026 | 1.65733 | 0.8810 | 0.365 | 2.760 |
| | 12 H | 21 | 21 | 1.1540 | 0.64955 | 56.3 | 0.9884 | 1.80366 | 1.0600 | 0.253 | 2.830 |
| | 14 H | 21 | 21 | 1.0487 | 0.47745 | 45.5 | 0.9301 | 1.71638 | 1.0400 | 0.213 | 2.020 |
| | 16 H | 21 | 21 | 1.0530 | 0.51872 | 49.3 | 0.9094 | 1.83140 | 0.9160 | 0.187 | 1.910 |
| | 18 H | 21 | 21 | 1.1267 | 0.52684 | 46.8 | 0.9840 | 1.80039 | 1.1600 | 0.194 | 2.030 |
| | 20 H | 21 | 21 | 1.1377 | 0.65876 | 57.9 | 0.9610 | 1.86589 | 1.0600 | 0.228 | 2.870 |
| | 22 H | 21 | 21 | 1.0311 | 0.51150 | 49.6 | 0.8924 | 1.82207 | 0.9360 | 0.190 | 2.160 |
| | 23.5 H | 21 | 21 | 0.9300 | 0.47474 | 51.0 | 0.7887 | 1.92430 | 0.8990 | 0.129 | 1.880 |

Application Site = Thigh

| | | | | | | | | | | | |
|---|---|---|---|---|---|---|---|---|---|---|---|
| Day 25 | 0 H | 11 | 11 | 0.5459 | 0.31177 | 57.1 | 0.4575 | 1.99998 | 0.5360 | 0.087 | 1.300 |
| | 4 H | 11 | 11 | 0.3757 | 0.18016 | 47.9 | 0.3262 | 1.89010 | 0.3440 | 0.062 | 0.709 |
| | 8 H | 11 | 11 | 0.4448 | 0.25511 | 57.4 | 0.3661 | 2.04796 | 0.4200 | 0.087 | 0.864 |
| | 12 H | 11 | 11 | 0.5095 | 0.30534 | 59.9 | 0.4246 | 1.96224 | 0.4230 | 0.105 | 1.160 |
| Day 26 | 0 H | 11 | 11 | 0.8664 | 0.48505 | 56.0 | 0.7679 | 1.64271 | 0.6140 | 0.398 | 1.950 |
| | 4 H | 11 | 11 | 0.5822 | 0.24421 | 41.9 | 0.5363 | 1.53465 | 0.4920 | 0.299 | 0.961 |
| | 8 H | 11 | 11 | 0.8017 | 0.50124 | 62.5 | 0.6800 | 1.81246 | 0.5910 | 0.280 | 1.850 |
| | 12 H | 11 | 11 | 0.8817 | 0.32211 | 36.5 | 0.8316 | 1.43050 | 0.8790 | 0.466 | 1.550 |
| Day 27 | 0 H | 9 | 9 | 0.7659 | 0.22662 | 29.6 | 0.7316 | 1.39657 | 0.8240 | 0.429 | 1.010 |
| | 1 H | 9 | 9 | 0.6961 | 0.22690 | 32.6 | 0.6580 | 1.45179 | 0.7110 | 0.337 | 0.998 |
| | 2 H | 9 | 9 | 0.6704 | 0.22674 | 33.8 | 0.6336 | 1.44547 | 0.6660 | 0.326 | 1.040 |
| | 4 H | 9 | 9 | 0.7407 | 0.55140 | 74.4 | 0.6006 | 1.95373 | 0.5670 | 0.234 | 1.970 |
| | 5 H | 9 | 9 | 0.6593 | 0.40489 | 61.4 | 0.5593 | 1.84149 | 0.4930 | 0.238 | 1.450 |
| | 6 H | 9 | 9 | 0.6381 | 0.59883 | 93.8 | 0.4821 | 2.10452 | 0.3660 | 0.215 | 2.120 |
| | 7 H | 9 | 9 | 0.6203 | 0.40515 | 65.3 | 0.5063 | 1.98142 | 0.4170 | 0.239 | 1.290 |
| | 8 H | 9 | 9 | 0.6990 | 0.50385 | 72.1 | 0.5434 | 2.14173 | 0.3970 | 0.244 | 1.440 |
| | 10 H | 9 | 9 | 0.6318 | 0.52626 | 83.3 | 0.4670 | 2.30320 | 0.4510 | 0.149 | 1.780 |
| | 12 H | 9 | 9 | 0.7850 | 0.51311 | 65.4 | 0.6231 | 2.13741 | 0.8430 | 0.220 | 1.690 |
| | 14 H | 9 | 9 | 0.8909 | 0.60851 | 68.3 | 0.7309 | 1.94514 | 0.7750 | 0.297 | 2.080 |
| | 16 H | 9 | 9 | 1.0051 | 0.49241 | 49.0 | 0.8930 | 1.69568 | 1.0200 | 0.441 | 1.690 |
| | 18 H | 9 | 9 | 0.9699 | 0.45472 | 46.9 | 0.8590 | 1.73615 | 1.1100 | 0.357 | 1.540 |
| | 20 H | 9 | 9 | 0.9627 | 0.52371 | 54.4 | 0.8240 | 1.87126 | 1.0600 | 0.273 | 1.940 |
| | 22 H | 9 | 9 | 0.7457 | 0.41963 | 56.3 | 0.6414 | 1.81633 | 0.6860 | 0.271 | 1.450 |
| | 23.5 H | 9 | 9 | 0.6751 | 0.30935 | 45.8 | 0.6187 | 1.54714 | 0.5870 | 0.374 | 1.260 |
| Day 28 | 4 H | 9 | 9 | 0.6501 | 0.33546 | 51.6 | 0.5898 | 1.57382 | 0.6110 | 0.306 | 1.450 |
| | 8 H | 9 | 9 | 0.9154 | 0.50750 | 55.4 | 0.7880 | 1.83625 | 0.8720 | 0.285 | 1.940 |
| | 12 H | 9 | 9 | 0.9857 | 0.53062 | 53.8 | 0.8741 | 1.67978 | 0.9300 | 0.398 | 2.140 |
| Day 29 | 0 H | 12 | 12 | 0.8323 | 0.49058 | 58.9 | 0.6904 | 1.98820 | 0.7905 | 0.185 | 1.930 |
| | 4 H | 12 | 12 | 0.5280 | 0.23157 | 43.9 | 0.4816 | 1.57708 | 0.4215 | 0.206 | 0.892 |
| | 8 H | 12 | 12 | 0.6492 | 0.37254 | 57.4 | 0.5579 | 1.79230 | 0.5430 | 0.200 | 1.340 |
| | 12 H | 12 | 12 | 0.7147 | 0.33321 | 46.6 | 0.6404 | 1.65449 | 0.6585 | 0.295 | 1.240 |
| Day 30 | 0 H | 11 | 11 | 0.8924 | 0.30631 | 34.3 | 0.8464 | 1.40745 | 0.8610 | 0.495 | 1.520 |
| | 1 H | 11 | 11 | 0.7264 | 0.35752 | 49.2 | 0.6565 | 1.59424 | 0.6090 | 0.348 | 1.530 |
| | 2 H | 11 | 11 | 0.6491 | 0.21364 | 32.9 | 0.6178 | 1.39147 | 0.6190 | 0.390 | 0.974 |
| | 4 H | 11 | 11 | 0.6865 | 0.33996 | 49.5 | 0.6107 | 1.67154 | 0.6760 | 0.307 | 1.210 |
| | 5 H | 11 | 11 | 0.7082 | 0.40994 | 57.9 | 0.6160 | 1.72547 | 0.5880 | 0.297 | 1.600 |
| | 6 H | 11 | 11 | 0.6519 | 0.33980 | 52.1 | 0.5638 | 1.80838 | 0.6090 | 0.212 | 1.250 |
| | 7 H | 11 | 11 | 0.7899 | 0.51478 | 65.2 | 0.6252 | 2.13852 | 0.7930 | 0.207 | 1.730 |
| | 8 H | 11 | 11 | 0.9367 | 0.58856 | 62.8 | 0.7401 | 2.17202 | 0.8650 | 0.233 | 1.780 |
| | 10 H | 11 | 11 | 0.7395 | 0.50770 | 68.7 | 0.5829 | 2.11814 | 0.6200 | 0.189 | 1.770 |
| | 12 H | 11 | 11 | 0.8881 | 0.77808 | 87.6 | 0.6636 | 2.20837 | 0.6830 | 0.218 | 2.900 |
| | 14 H | 11 | 11 | 1.0653 | 0.92586 | 86.9 | 0.8037 | 2.17880 | 0.8560 | 0.258 | 3.450 |
| | 16 H | 11 | 11 | 0.9864 | 0.51673 | 52.4 | 0.8481 | 1.85590 | 0.9840 | 0.245 | 2.040 |
| | 18 H | 11 | 11 | 1.1151 | 0.65786 | 59.0 | 0.9251 | 2.00820 | 1.0400 | 0.207 | 2.620 |
| | 20 H | 11 | 11 | 1.1024 | 0.60757 | 55.1 | 0.9228 | 1.99273 | 1.1800 | 0.211 | 2.380 |
| | 22 H | 11 | 11 | 0.9143 | 0.60935 | 66.6 | 0.7273 | 2.11695 | 0.7180 | 0.177 | 2.040 |
| | 23.5 H | 11 | 11 | 0.7845 | 0.43513 | 55.5 | 0.6564 | 1.98755 | 0.6800 | 0.152 | 1.590 |
| Days | 0 H | 20 | 20 | 0.8355 | 0.27418 | 32.8 | 0.7927 | 1.40162 | 0.8425 | 0.429 | 1.520 |
| 27 & 30 | 1 H | 20 | 20 | 0.7128 | 0.29865 | 41.9 | 0.6572 | 1.51580 | 0.6775 | 0.337 | 1.530 |
| Combined | 2 H | 20 | 20 | 0.6587 | 0.21398 | 32.5 | 0.6249 | 1.40321 | 0.6350 | 0.326 | 1.040 |
| | 4 H | 20 | 20 | 0.7109 | 0.43544 | 61.3 | 0.6061 | 1.77284 | 0.6215 | 0.234 | 1.970 |
| | 5 H | 20 | 20 | 0.6862 | 0.39761 | 57.9 | 0.5898 | 1.75445 | 0.5405 | 0.238 | 1.600 |
| | 6 H | 20 | 20 | 0.6457 | 0.46023 | 71.3 | 0.5254 | 1.91808 | 0.5745 | 0.212 | 2.120 |
| | 7 H | 20 | 20 | 0.7136 | 0.46484 | 65.1 | 0.5686 | 2.04611 | 0.6965 | 0.207 | 1.730 |
| | 8 H | 20 | 20 | 0.8298 | 0.55130 | 66.4 | 0.6440 | 2.14976 | 0.7915 | 0.233 | 1.780 |
| | 10 H | 20 | 20 | 0.6910 | 0.50527 | 73.1 | 0.5275 | 2.17303 | 0.5420 | 0.149 | 1.780 |
| | 12 H | 20 | 20 | 0.8417 | 0.65746 | 78.1 | 0.6451 | 2.13366 | 0.6960 | 0.218 | 2.900 |
| | 14 H | 20 | 20 | 0.9868 | 0.78422 | 79.5 | 0.7701 | 2.03948 | 0.8180 | 0.258 | 3.450 |
| | 16 H | 20 | 20 | 0.9948 | 0.49266 | 49.5 | 0.8681 | 1.75967 | 0.9970 | 0.245 | 2.040 |
| | 18 H | 20 | 20 | 1.0498 | 0.56598 | 53.9 | 0.8947 | 1.86048 | 1.0750 | 0.207 | 2.620 |
| | 20 H | 20 | 20 | 1.0395 | 0.56111 | 54.0 | 0.8769 | 1.91021 | 1.1200 | 0.211 | 2.380 |
| | 22 H | 20 | 20 | 0.8384 | 0.52628 | 62.8 | 0.6873 | 1.95602 | 0.7020 | 0.177 | 2.040 |
| | 23.5 H | 20 | 20 | 0.7353 | 0.37824 | 51.4 | 0.6392 | 1.77529 | 0.6685 | 0.152 | 1.590 |

Application Site = Abdomen

| | | | | | | | | | | | |
|---|---|---|---|---|---|---|---|---|---|---|---|
| Day 25 | 0 H | 12 | 12 | 0.8559 | 0.38007 | 44.4 | 0.7614 | 1.72897 | 0.7580 | 0.224 | 1.340 |
| | 4 H | 12 | 12 | 0.5725 | 0.33593 | 58.7 | 0.4801 | 1.94032 | 0.5070 | 0.109 | 1.340 |
| | 8 H | 12 | 12 | 0.6619 | 0.49938 | 75.4 | 0.5231 | 2.02481 | 0.4540 | 0.180 | 1.810 |
| | 12 H | 11 | 11 | 0.7678 | 0.59324 | 77.3 | 0.5923 | 2.15139 | 0.6440 | 0.208 | 2.210 |
| Day 26 | 0 H | 11 | 11 | 1.0170 | 0.65848 | 64.7 | 0.8238 | 2.04230 | 1.0900 | 0.223 | 2.470 |
| | 4 H | 11 | 11 | 0.9453 | 0.61240 | 64.8 | 0.7860 | 1.90388 | 0.7580 | 0.267 | 2.300 |
| | 8 H | 11 | 11 | 0.9732 | 0.64445 | 66.2 | 0.7493 | 2.27998 | 0.9140 | 0.151 | 1.980 |
| | 12 H | 11 | 11 | 1.0673 | 0.66268 | 62.1 | 0.8841 | 1.97633 | 0.8750 | 0.209 | 2.580 |
| Day 27 | 0 H | 11 | 11 | 0.9265 | 0.51511 | 55.6 | 0.8158 | 1.67838 | 0.7240 | 0.432 | 1.960 |
| | 1 H | 11 | 11 | 0.6864 | 0.28320 | 41.3 | 0.6332 | 1.53213 | 0.6280 | 0.333 | 1.200 |
| | 2 H | 11 | 11 | 0.6846 | 0.34457 | 50.3 | 0.6064 | 1.69019 | 0.5580 | 0.275 | 1.270 |
| | 4 H | 11 | 11 | 0.9067 | 0.66590 | 73.4 | 0.7424 | 1.89613 | 0.7080 | 0.338 | 2.570 |
| | 5 H | 11 | 11 | 0.8715 | 0.69574 | 79.8 | 0.7018 | 1.94180 | 0.7360 | 0.286 | 2.750 |
| | 6 H | 11 | 11 | 0.8515 | 0.60073 | 70.5 | 0.7041 | 1.87712 | 0.6780 | 0.283 | 2.340 |
| | 7 H | 11 | 11 | 0.9251 | 0.85810 | 92.8 | 0.7189 | 1.98763 | 0.6800 | 0.304 | 3.350 |
| | 8 H | 11 | 11 | 0.8564 | 0.52352 | 61.1 | 0.7264 | 1.88449 | 0.8360 | 0.173 | 2.210 |
| | 10 H | 11 | 11 | 0.9087 | 0.47489 | 52.3 | 0.8157 | 1.60445 | 0.7660 | 0.468 | 1.900 |
| | 12 H | 11 | 11 | 0.9115 | 0.39041 | 42.8 | 0.8482 | 1.47127 | 0.7630 | 0.498 | 1.820 |
| | 14 H | 11 | 11 | 0.9315 | 0.49896 | 53.6 | 0.8403 | 1.58290 | 0.8160 | 0.388 | 2.260 |
| | 16 H | 11 | 11 | 1.0727 | 0.42625 | 39.7 | 1.0035 | 1.45818 | 0.9040 | 0.613 | 1.960 |
| | 18 H | 11 | 11 | 1.0615 | 0.35591 | 33.5 | 1.0055 | 1.42014 | 1.0800 | 0.606 | 1.620 |
| | 20 H | 11 | 11 | 1.0844 | 0.41945 | 38.7 | 1.0182 | 1.44293 | 0.9620 | 0.584 | 1.980 |
| | 22 H | 11 | 11 | 1.0067 | 0.48036 | 47.7 | 0.9105 | 1.59773 | 0.8040 | 0.463 | 1.880 |
| | 23.5 H | 11 | 11 | 0.9287 | 0.59391 | 63.9 | 0.8065 | 1.69713 | 0.7200 | 0.421 | 2.500 |
| Day 28 | 4 H | 10 | 10 | 0.6127 | 0.25358 | 41.4 | 0.5715 | 1.47709 | 0.5975 | 0.284 | 1.220 |
| | 8 H | 10 | 10 | 0.6547 | 0.35608 | 54.4 | 0.5802 | 1.65843 | 0.4790 | 0.332 | 1.350 |
| | 12 H | 10 | 10 | 0.8782 | 0.45510 | 51.8 | 0.7665 | 1.76318 | 0.9055 | 0.366 | 1.670 |
| Day 29 | 0 H | 10 | 10 | 1.0294 | 0.43246 | 42.0 | 0.9445 | 1.56797 | 0.9940 | 0.423 | 1.780 |
| | 4 H | 10 | 10 | 0.6672 | 0.38443 | 57.6 | 0.5958 | 1.60208 | 0.5485 | 0.356 | 1.620 |
| | 8 H | 10 | 10 | 0.8180 | 0.42471 | 51.9 | 0.7379 | 1.59032 | 0.6350 | 0.373 | 1.780 |
| | 12 H | 10 | 10 | 0.9772 | 0.62645 | 64.1 | 0.8403 | 1.73878 | 0.7780 | 0.458 | 2.380 |
| Day 30 | 0 H | 9 | 9 | 0.6223 | 0.35642 | 57.3 | 0.5340 | 1.83375 | 0.4960 | 0.170 | 1.220 |
| | 1 H | 9 | 9 | 0.5937 | 0.41692 | 70.2 | 0.4753 | 2.04532 | 0.4740 | 0.165 | 1.300 |
| | 2 H | 9 | 9 | 0.5682 | 0.35033 | 61.7 | 0.4613 | 2.05524 | 0.4990 | 0.182 | 1.090 |
| | 4 H | 9 | 9 | 0.5613 | 0.36307 | 64.7 | 0.4531 | 2.06279 | 0.5990 | 0.158 | 1.190 |
| | 5 H | 9 | 9 | 0.5039 | 0.26008 | 51.6 | 0.4307 | 1.89151 | 0.5850 | 0.151 | 0.794 |
| | 6 H | 9 | 9 | 0.4732 | 0.25628 | 54.2 | 0.4137 | 1.74385 | 0.3460 | 0.178 | 0.851 |
| | 7 H | 9 | 9 | 0.5576 | 0.23738 | 42.6 | 0.5042 | 1.66077 | 0.5430 | 0.199 | 0.899 |
| | 8 H | 9 | 9 | 0.6087 | 0.21507 | 35.3 | 0.5734 | 1.45199 | 0.5930 | 0.326 | 0.880 |
| | 10 H | 9 | 9 | 0.5918 | 0.18376 | 31.1 | 0.5631 | 1.41664 | 0.6020 | 0.285 | 0.847 |
| | 12 H | 9 | 9 | 0.5992 | 0.24044 | 40.1 | 0.5619 | 1.44860 | 0.5450 | 0.353 | 1.020 |
| | 14 H | 9 | 9 | 0.6353 | 0.22602 | 35.6 | 0.6032 | 1.40225 | 0.6200 | 0.347 | 1.120 |
| | 16 H | 9 | 9 | 0.7680 | 0.27706 | 36.1 | 0.7242 | 1.44498 | 0.6800 | 0.370 | 1.290 |
| | 18 H | 9 | 9 | 0.7886 | 0.30936 | 39.2 | 0.7428 | 1.42577 | 0.6800 | 0.495 | 1.320 |
| | 20 H | 9 | 9 | 0.6752 | 0.18458 | 27.3 | 0.6532 | 1.31491 | 0.6320 | 0.435 | 0.971 |
| | 22 H | 9 | 9 | 0.6586 | 0.27135 | 41.2 | 0.6128 | 1.49041 | 0.5530 | 0.365 | 1.190 |
| | 23.5 H | 9 | 9 | 0.6134 | 0.32518 | 53.0 | 0.5450 | 1.66356 | 0.4150 | 0.282 | 1.210 |
| Days | 0 H | 20 | 20 | 0.7896 | 0.46609 | 59.0 | 0.6742 | 1.79574 | 0.6340 | 0.170 | 1.960 |
| 27 & 30 | 1 H | 20 | 20 | 0.6446 | 0.34299 | 53.2 | 0.5565 | 1.78054 | 0.5870 | 0.165 | 1.300 |
| Combined | 2 H | 20 | 20 | 0.6323 | 0.34307 | 54.3 | 0.5362 | 1.85679 | 0.5390 | 0.182 | 1.270 |
| | 4 H | 20 | 20 | 0.7513 | 0.56565 | 75.3 | 0.5945 | 2.02775 | 0.6065 | 0.158 | 2.570 |
| | 5 H | 20 | 20 | 0.7061 | 0.56431 | 79.9 | 0.5634 | 1.97759 | 0.6785 | 0.151 | 2.750 |
| | 6 H | 20 | 20 | 0.6813 | 0.50486 | 74.1 | 0.5543 | 1.90097 | 0.5005 | 0.178 | 2.340 |
| | 7 H | 20 | 20 | 0.7597 | 0.66818 | 88.0 | 0.6129 | 1.86655 | 0.6615 | 0.199 | 3.350 |
| | 8 H | 20 | 20 | 0.7449 | 0.42392 | 56.9 | 0.6531 | 1.70461 | 0.7465 | 0.173 | 2.210 |
| | 10 H | 20 | 20 | 0.7661 | 0.39885 | 52.1 | 0.6904 | 1.57175 | 0.7060 | 0.285 | 1.900 |
| | 12 H | 20 | 20 | 0.7710 | 0.36052 | 46.8 | 0.7048 | 1.52931 | 0.6750 | 0.353 | 1.820 |
| | 14 H | 20 | 20 | 0.7983 | 0.41881 | 52.5 | 0.7238 | 1.54236 | 0.6715 | 0.347 | 2.260 |
| | 16 H | 20 | 20 | 0.9356 | 0.39005 | 41.7 | 0.8665 | 1.49115 | 0.8085 | 0.370 | 1.960 |
| | 18 H | 20 | 20 | 0.9387 | 0.35548 | 37.9 | 0.8774 | 1.45693 | 0.7955 | 0.495 | 1.620 |
| | 20 H | 20 | 20 | 0.9003 | 0.38801 | 43.1 | 0.8338 | 1.47996 | 0.7910 | 0.435 | 1.980 |
| | 22 H | 20 | 20 | 0.8501 | 0.42899 | 50.5 | 0.7619 | 1.60433 | 0.7700 | 0.365 | 1.880 |
| | 23.5 H | 20 | 20 | 0.7869 | 0.50603 | 64.3 | 0.6761 | 1.72350 | 0.6775 | 0.282 | 2.500 |

Application Site = Flank

| | | | | | | | | | | | |
|---|---|---|---|---|---|---|---|---|---|---|---|
| Day 25 | 0 H | 9 | 9 | 0.6722 | 0.29287 | 43.6 | 0.6120 | 1.61551 | 0.6290 | 0.238 | 1.170 |
| | 4 H | 9 | 9 | 0.7684 | 0.52305 | 68.1 | 0.6275 | 2.02705 | 0.7370 | 0.159 | 1.980 |
| | 8 H | 9 | 9 | 1.0453 | 0.73978 | 70.8 | 0.8723 | 1.85081 | 0.7130 | 0.376 | 2.780 |
| | 12 H | 9 | 9 | 1.1301 | 0.57397 | 50.8 | 1.0344 | 1.52769 | 0.9720 | 0.565 | 2.530 |
| Day 26 | 0 H | 10 | 10 | 0.5991 | 0.51432 | 85.8 | 0.4750 | 1.92537 | 0.4065 | 0.239 | 1.810 |
| | 4 H | 10 | 10 | 0.7246 | 0.66163 | 91.3 | 0.5297 | 2.21190 | 0.3775 | 0.232 | 2.050 |
| | 8 H | 10 | 10 | 0.8006 | 0.65923 | 82.3 | 0.5965 | 2.24353 | 0.5675 | 0.219 | 2.120 |
| | 12 H | 10 | 10 | 0.8713 | 0.55705 | 63.9 | 0.6991 | 2.11591 | 0.7015 | 0.167 | 1.860 |
| Day 27 | 0 H | 12 | 12 | 1.0206 | 0.59848 | 58.6 | 0.8713 | 1.81989 | 0.9135 | 0.339 | 2.190 |
| | 1 H | 12 | 12 | 0.8854 | 0.48531 | 54.8 | 0.7686 | 1.76217 | 0.8550 | 0.321 | 1.900 |
| | 2 H | 12 | 12 | 0.7239 | 0.32666 | 45.1 | 0.6551 | 1.61715 | 0.6995 | 0.272 | 1.320 |
| | 4 H | 12 | 12 | 0.7117 | 0.41155 | 57.8 | 0.6366 | 1.59337 | 0.6350 | 0.335 | 1.890 |
| | 5 H | 12 | 12 | 0.7970 | 0.61097 | 76.7 | 0.6506 | 1.88533 | 0.5595 | 0.276 | 2.460 |
| | 6 H | 12 | 12 | 0.8569 | 0.81012 | 94.5 | 0.5915 | 2.51432 | 0.7135 | 0.145 | 3.070 |
| | 7 H | 12 | 12 | 0.9963 | 0.92756 | 93.1 | 0.6780 | 2.54736 | 0.6695 | 0.170 | 3.350 |
| | 8 H | 12 | 12 | 1.0207 | 0.95022 | 93.1 | 0.7304 | 2.34634 | 0.7845 | 0.177 | 3.600 |
| | 10 H | 12 | 12 | 1.1392 | 0.92575 | 81.3 | 0.8959 | 2.02851 | 1.0305 | 0.333 | 3.700 |
| | 12 H | 12 | 12 | 1.0202 | 0.94429 | 92.6 | 0.7286 | 2.34821 | 0.6125 | 0.173 | 3.530 |
| | 14 H | 12 | 12 | 1.3253 | 1.05047 | 79.3 | 0.9441 | 2.56498 | 0.9325 | 0.123 | 3.630 |
| | 16 H | 12 | 12 | 1.3274 | 0.89040 | 67.1 | 1.0127 | 2.32638 | 1.2750 | 0.185 | 2.830 |
| | 18 H | 12 | 12 | 1.1423 | 0.67794 | 59.4 | 0.8945 | 2.30262 | 1.1350 | 0.194 | 2.500 |
| | 20 H | 12 | 12 | 1.3704 | 0.96508 | 70.4 | 1.0214 | 2.41068 | 1.2950 | 0.198 | 3.380 |
| | 22 H | 12 | 12 | 1.2376 | 1.10689 | 89.4 | 0.9098 | 2.23945 | 0.9500 | 0.339 | 4.180 |
| | 23.5 H | 12 | 12 | 1.1897 | 1.30093 | 109.4 | 0.7457 | 2.70731 | 0.6040 | 0.170 | 4.610 |
| Day 28 | 4 H | 12 | 12 | 0.9311 | 1.07976 | 116.0 | 0.6766 | 2.05352 | 0.5450 | 0.323 | 4.230 |
| | 8 H | 12 | 12 | 0.9149 | 0.90740 | 99.2 | 0.6574 | 2.30859 | 0.6595 | 0.143 | 3.540 |
| | 12 H | 12 | 12 | 0.8508 | 0.60145 | 70.7 | 0.7159 | 1.81436 | 0.8040 | 0.274 | 2.580 |
| Day 29 | 0 H | 9 | 9 | 0.7540 | 0.35840 | 47.5 | 0.6774 | 1.64381 | 0.6460 | 0.386 | 1.290 |
| | 4 H | 9 | 9 | 0.5993 | 0.24232 | 40.4 | 0.5506 | 1.58457 | 0.6230 | 0.224 | 1.010 |
| | 8 H | 9 | 9 | 0.6587 | 0.31488 | 47.8 | 0.5870 | 1.69309 | 0.5640 | 0.245 | 1.090 |
| | 12 H | 9 | 9 | 0.8814 | 0.36184 | 41.1 | 0.8202 | 1.50138 | 0.8640 | 0.383 | 1.680 |
| Day 30 | 0 H | 11 | 11 | 0.9399 | 0.40036 | 42.6 | 0.8191 | 1.89480 | 1.0100 | 0.166 | 1.380 |
| | 1 H | 11 | 11 | 0.7815 | 0.34249 | 43.8 | 0.6955 | 1.73588 | 0.8460 | 0.208 | 1.270 |
| | 2 H | 11 | 11 | 0.8638 | 0.37218 | 43.1 | 0.7512 | 1.89862 | 0.9500 | 0.162 | 1.410 |
| | 4 H | 11 | 11 | 0.7585 | 0.33664 | 44.4 | 0.6662 | 1.80852 | 0.8520 | 0.191 | 1.280 |
| | 5 H | 11 | 11 | 0.6801 | 0.29523 | 43.4 | 0.6189 | 1.59817 | 0.7060 | 0.300 | 1.140 |
| | 6 H | 11 | 11 | 0.7087 | 0.34400 | 48.5 | 0.6279 | 1.71641 | 0.6490 | 0.256 | 1.350 |
| | 7 H | 11 | 11 | 0.8363 | 0.62511 | 74.7 | 0.6834 | 1.91810 | 0.6000 | 0.210 | 2.470 |
| | 8 H | 11 | 11 | 0.8677 | 0.36331 | 41.9 | 0.7956 | 1.56690 | 0.8140 | 0.386 | 1.370 |
| | 10 H | 11 | 11 | 0.8692 | 0.36211 | 41.7 | 0.8047 | 1.50878 | 0.8050 | 0.435 | 1.600 |
| | 12 H | 11 | 11 | 0.8829 | 0.40916 | 46.3 | 0.8109 | 1.52540 | 0.7310 | 0.461 | 1.860 |
| | 14 H | 11 | 11 | 1.1887 | 0.52014 | 43.8 | 1.0588 | 1.73139 | 1.2000 | 0.332 | 2.030 |
| | 16 H | 11 | 11 | 1.0951 | 0.48695 | 44.5 | 0.9871 | 1.64244 | 0.9930 | 0.447 | 1.750 |
| | 18 H | 11 | 11 | 1.3349 | 0.70412 | 52.7 | 1.1748 | 1.71481 | 1.2700 | 0.491 | 2.660 |
| | 20 H | 11 | 11 | 1.1201 | 0.57443 | 51.3 | 0.9911 | 1.70965 | 1.0400 | 0.340 | 2.470 |
| | 22 H | 11 | 11 | 0.9278 | 0.48847 | 52.6 | 0.8199 | 1.69467 | 0.7730 | 0.334 | 2.020 |
| | 23.5 H | 11 | 11 | 1.0127 | 0.55687 | 55.0 | 0.8877 | 1.73559 | 1.0000 | 0.283 | 2.370 |
| Days | 0 H | 23 | 23 | 0.9820 | 0.50364 | 51.3 | 0.8460 | 1.83111 | 1.0000 | 0.166 | 2.190 |
| 27 & 30 | 1 H | 23 | 23 | 0.8357 | 0.41702 | 49.9 | 0.7327 | 1.73144 | 0.8460 | 0.208 | 1.900 |
| Combined | 2 H | 23 | 23 | 0.7908 | 0.34845 | 44.1 | 0.6994 | 1.74072 | 0.8080 | 0.162 | 1.410 |
| | 4 H | 23 | 23 | 0.7341 | 0.36982 | 50.4 | 0.6506 | 1.67916 | 0.7390 | 0.191 | 1.890 |
| | 5 H | 23 | 23 | 0.7411 | 0.47940 | 64.7 | 0.6352 | 1.73186 | 0.6050 | 0.276 | 2.460 |
| | 6 H | 23 | 23 | 0.7860 | 0.62263 | 79.2 | 0.6087 | 2.11154 | 0.6490 | 0.145 | 3.070 |
| | 7 H | 23 | 23 | 0.9197 | 0.78389 | 85.2 | 0.6806 | 2.21165 | 0.6000 | 0.170 | 3.350 |
| | 8 H | 23 | 23 | 0.9475 | 0.71942 | 75.9 | 0.7609 | 1.96642 | 0.8140 | 0.177 | 3.600 |
| | 10 H | 23 | 23 | 1.0100 | 0.71213 | 70.5 | 0.8511 | 1.77622 | 0.9110 | 0.333 | 3.700 |
| | 12 H | 23 | 23 | 0.9545 | 0.72584 | 76.0 | 0.7669 | 1.95351 | 0.7170 | 0.173 | 3.530 |
| | 14 H | 23 | 23 | 1.2600 | 0.82437 | 65.4 | 0.9973 | 2.14732 | 1.1700 | 0.123 | 3.630 |
| | 16 H | 23 | 23 | 1.2163 | 0.71991 | 59.2 | 1.0004 | 1.98274 | 1.1900 | 0.185 | 2.830 |
| | 18 H | 23 | 23 | 1.2344 | 0.68179 | 55.2 | 1.0190 | 2.02726 | 1.1900 | 0.194 | 2.660 |
| | 20 H | 23 | 23 | 1.2507 | 0.79500 | 63.6 | 1.0068 | 2.05398 | 1.2400 | 0.198 | 3.380 |
| | 22 H | 23 | 23 | 1.0894 | 0.86377 | 79.3 | 0.8656 | 1.96211 | 0.7730 | 0.334 | 4.180 |
| | 23.5 H | 23 | 23 | 1.1050 | 0.99766 | 90.3 | 0.8105 | 2.22842 | 0.7140 | 0.170 | 4.610 |

Summary statistics for AUC_{O-t,ss} and C_{max,ss} for unconjugated rotigotine for each patch application site using separated data for Day 27 and Day 30 are given in Table 17.

TABLE 17
Descriptive statistics of parameters of pharmacokinetics for rotigotine under multiple
dose in patients with early-stage Parkinson's disease (H = hip, S = shoulder,
UA = upper arm, T = thigh, AB = abdomen, F = flank)

| Day Parameter | Site | n | Mean | SD | CV (%) | Geo. Mean | Geo. SD | Median | Min | Max |
|---|---|---|---|---|---|---|---|---|---|---|
| Day 27 | | | | | | | | | | |
| AUC 0-t, ss | H | 10 | 24.714 | 12.1148 | 49.0 | 22.284 | 1.6080 | 18.704 | 11.27 | 45.38 |
| (ng * h/ml) | S | 11 | 21.147 | 9.9209 | 46.9 | 19.167 | 1.6046 | 17.603 | 7.51 | 43.51 |
| | UA | 10 | 23.846 | 12.6658 | 53.1 | 20.347 | 1.9076 | 20.963 | 5.13 | 46.50 |
| | T | 9 | 18.464 | 10.0154 | 54.2 | 15.994 | 1.7917 | 17.649 | 7.06 | 34.06 |
| | AB | 11 | 21.868 | 9.9971 | 45.7 | 20.228 | 1.4849 | 16.608 | 13.72 | 45.82 |
| | F | 12 | 25.438 | 17.4836 | 68.7 | 20.817 | 1.9421 | 20.727 | 6.73 | 68.64 |
| AUC 0-t, ss, | H | 10 | 263.38 | 132.896 | 50.5 | 239.15 | 1.567 | 231.03 | 125.6 | 581.9 |
| normalized | S | 11 | 238.37 | 75.596 | 31.7 | 225.55 | 1.444 | 262.41 | 106.6 | 354.1 |
| (ng * h * kg/ml/mg) | UA | 10 | 322.54 | 99.970 | 31.0 | 308.16 | 1.384 | 298.42 | 165.4 | 497.0 |
| | T | 9 | 222.62 | 84.993 | 38.2 | 207.97 | 1.488 | 227.07 | 118.7 | 372.0 |
| | AB | 11 | 316.61 | 233.860 | 73.9 | 262.54 | 1.859 | 232.01 | 89.3 | 952.1 |
| | F | 12 | 258.09 | 132.295 | 51.3 | 233.42 | 1.575 | 218.30 | 104.3 | 578.1 |
| Maximum | H | 10 | 1.8159 | 1.19253 | 65.7 | 1.5354 | 1.80840 | 1.2250 | 0.679 | 4.440 |
| Concentration | S | 11 | 1.3583 | 0.57413 | 42.3 | 1.2418 | 1.59520 | 1.1900 | 0.431 | 2.580 |
| (ng/ml) | UA | 10 | 1.4986 | 0.71726 | 47.9 | 1.3274 | 1.74189 | 1.4000 | 0.403 | 2.870 |
| | T | 9 | 1.1772 | 0.61214 | 52.0 | 1.0354 | 1.73314 | 1.1800 | 0.469 | 2.120 |
| | AB | 11 | 1.5598 | 0.81231 | 52.1 | 1.3953 | 1.62515 | 1.2100 | 0.775 | 3.350 |
| | F | 12 | 1.9218 | 1.10854 | 57.7 | 1.6674 | 1.74328 | 1.6500 | 0.683 | 4.610 |
| Maximum Conc | H | 10 | 19.784 | 15.6559 | 79.1 | 16.478 | 1.7821 | 13.943 | 9.46 | 61.31 |
| normalized | S | 11 | 15.493 | 5.0158 | 32.4 | 14.613 | 1.4647 | 15.873 | 6.12 | 22.37 |
| (ng * kg/ml/mg) | UA | 10 | 21.552 | 8.7051 | 40.4 | 20.103 | 1.4770 | 19.225 | 11.16 | 39.01 |
| | T | 9 | 14.217 | 4.9198 | 34.6 | 13.464 | 1.4247 | 14.659 | 7.69 | 23.15 |
| | AB | 11 | 21.413 | 12.9134 | 60.3 | 18.110 | 1.8837 | 20.181 | 4.96 | 52.52 |
| | F | 12 | 20.022 | 8.2775 | 41.3 | 18.697 | 1.4562 | 16.847 | 10.58 | 38.83 |
| Average | H | 10 | 1.0516 | 0.51553 | 49.0 | 0.9483 | 1.60805 | 0.7959 | 0.479 | 1.931 |
| Concentration | S | 11 | 0.8999 | 0.42217 | 46.9 | 0.8156 | 1.60465 | 0.7491 | 0.319 | 1.852 |
| (ng/ml) | UA | 10 | 1.0147 | 0.53897 | 53.1 | 0.8658 | 1.90761 | 0.8920 | 0.218 | 1.979 |
| | T | 9 | 0.7857 | 0.42619 | 54.2 | 0.6806 | 1.79171 | 0.7510 | 0.300 | 1.449 |
| | AB | 11 | 0.9306 | 0.42541 | 45.7 | 0.8608 | 1.48493 | 0.7067 | 0.584 | 1.950 |
| | F | 12 | 1.0825 | 0.74398 | 68.7 | 0.8858 | 1.94211 | 0.8820 | 0.286 | 2.921 |
| Time to Maximum | H | 10 | 13.50 | 7.382 | 54.7 | 13.42 | 1.758 | 16.00 | 0.0 | 22.0 |
| Concentration | S | 11 | 13.45 | 7.699 | 57.2 | 15.65 | 1.456 | 18.00 | 0.0 | 20.0 |
| (hours) | UA | 10 | 9.60 | 9.324 | 97.1 | 14.97 | 1.520 | 9.00 | 0.0 | 22.0 |
| | T | 9 | 12.00 | 7.874 | 65.6 | 14.57 | 1.503 | 16.00 | 0.0 | 20.0 |
| | AB | 11 | 12.95 | 8.020 | 61.9 | 11.82 | 2.124 | 16.00 | 0.0 | 23.5 |
| | F | 12 | 13.71 | 8.516 | 62.1 | 11.65 | 2.478 | 16.00 | 0.0 | 23.5 |
| Peak-Trough | H | 10 | 114.3 | 43.18 | 37.8 | 107.3 | 1.45 | 104.2 | 61 | 198 |
| Fluctuation (%) | S | 11 | 99.6 | 21.06 | 21.2 | 97.6 | 1.24 | 102.9 | 72 | 136 |
| | UA | 10 | 94.5 | 34.06 | 36.1 | 89.3 | 1.43 | 87.1 | 51 | 168 |
| | T | 9 | 95.0 | 24.11 | 25.4 | 92.0 | 1.32 | 105.9 | 57 | 124 |
| | AB | 11 | 116.6 | 70.07 | 60.1 | 104.7 | 1.56 | 88.3 | 60 | 314 |
| | F | 12 | 148.1 | 36.34 | 24.5 | 144.1 | 1.28 | 137.9 | 100 | 200 |
| Half Value | H | 10 | 16.39 | 6.414 | 39.1 | 14.87 | 1.670 | 17.67 | 4.9 | 23.5 |
| Duration (hour) | S | 11 | 18.35 | 4.227 | 23.0 | 17.86 | 1.287 | 19.61 | 11.7 | 23.0 |
| | UA | 10 | 19.34 | 3.802 | 19.7 | 18.98 | 1.230 | 19.83 | 13.1 | 23.5 |
| | T | 9 | 18.42 | 3.690 | 20.0 | 18.10 | 1.217 | 16.05 | 14.7 | 23.5 |
| | AB | 11 | 17.93 | 6.295 | 35.1 | 15.05 | 2.339 | 20.45 | 1.2 | 23.5 |
| | F | 12 | 11.42 | 4.479 | 39.2 | 10.57 | 1.530 | 10.84 | 4.5 | 18.3 |
| Apparent Dose of | H | 10 | 6.861 | 2.0513 | 29.9 | 6.590 | 1.3500 | 6.305 | 4.12 | 10.33 |
| Rotigotine (mg) | S | 11 | 6.474 | 2.0421 | 31.5 | 6.155 | 1.4096 | 6.500 | 3.53 | 9.68 |
| | UA | 10 | 5.619 | 2.7460 | 48.9 | 4.824 | 1.9268 | 5.535 | 1.06 | 10.10 |
| | T | 9 | 6.438 | 2.9351 | 45.6 | 5.912 | 1.5345 | 5.130 | 3.48 | 11.57 |
| | AB | 11 | 6.369 | 2.4858 | 39.0 | 5.773 | 1.6789 | 7.090 | 1.61 | 9.38 |
| | F | 12 | 7.258 | 2.6769 | 36.9 | 6.883 | 1.3880 | 6.170 | 4.83 | 13.52 |
| Day 30 | | | | | | | | | | |
| AUC 0-t, ss | H | 10 | 22.916 | 13.4155 | 58.5 | 19.957 | 1.7352 | 19.954 | 8.49 | 54.54 |
| (ng * h/ml) | S | 11 | 26.442 | 13.8860 | 52.5 | 23.741 | 1.6054 | 22.634 | 12.33 | 57.77 |
| | UA | 11 | 22.333 | 8.9481 | 40.1 | 20.795 | 1.4861 | 21.025 | 11.85 | 39.68 |
| | T | 11 | 20.713 | 11.8144 | 57.0 | 17.832 | 1.7938 | 18.354 | 7.37 | 46.16 |
| | AB | 9 | 14.776 | 5.2155 | 35.3 | 13.964 | 1.4308 | 14.797 | 8.85 | 23.22 |
| | F | 11 | 22.577 | 8.9371 | 39.6 | 20.789 | 1.5642 | 25.736 | 9.14 | 39.98 |
| AUC 0-t, ss, | H | 10 | 272.01 | 119.459 | 43.9 | 251.27 | 1.510 | 229.22 | 143.0 | 489.0 |
| normalized | S | 11 | 239.26 | 102.668 | 42.9 | 219.73 | 1.553 | 206.37 | 93.3 | 421.7 |
| (ng * h * kg/ml/mg) | UA | 11 | 218.21 | 50.674 | 23.2 | 212.44 | 1.284 | 218.78 | 121.8 | 299.8 |
| | T | 11 | 275.09 | 158.016 | 57.4 | 248.30 | 1.551 | 235.76 | 129.6 | 717.8 |
| | AB | 9 | 224.48 | 49.363 | 22.0 | 219.75 | 1.245 | 232.90 | 152.7 | 319.7 |
| | F | 11 | 255.07 | 110.435 | 43.3 | 233.60 | 1.561 | 218.91 | 118.3 | 439.1 |
| Maximum | H | 10 | 1.7803 | 1.40713 | 79.0 | 1.4347 | 1.94153 | 1.3150 | 0.598 | 5.310 |
| Concentration | S | 11 | 1.8044 | 1.20853 | 67.0 | 1.5544 | 1.70345 | 1.3900 | 0.874 | 4.810 |
| (ng/ml) | UA | 11 | 1.4595 | 0.63351 | 43.4 | 1.3509 | 1.49873 | 1.2900 | 0.747 | 2.830 |
| | T | 11 | 1.4849 | 0.78230 | 52.7 | 1.3303 | 1.62641 | 1.2500 | 0.555 | 3.450 |
| | AB | 9 | 0.9682 | 0.31038 | 32.1 | 0.9190 | 1.42608 | 1.0100 | 0.502 | 1.320 |
| | F | 11 | 1.5509 | 0.71145 | 45.9 | 1.3920 | 1.66366 | 1.4300 | 0.583 | 2.660 |
| Maximum Conc | H | 10 | 20.128 | 11.2346 | 55.8 | 18.063 | 1.5895 | 15.754 | 10.08 | 47.61 |
| normalized | S | 11 | 16.303 | 8.9019 | 54.6 | 14.386 | 1.6858 | 14.201 | 5.60 | 35.11 |
| (ng * kg/ml/mg) | UA | 11 | 14.249 | 3.5763 | 25.1 | 13.801 | 1.3154 | 14.711 | 7.68 | 19.46 |
| | T | 11 | 20.719 | 12.0527 | 58.2 | 18.523 | 1.5976 | 16.694 | 8.59 | 53.65 |
| | AB | 9 | 14.874 | 4.0113 | 27.0 | 14.462 | 1.2751 | 13.548 | 11.17 | 23.78 |
| | F | 11 | 17.834 | 9.3068 | 52.2 | 15.642 | 1.7272 | 16.666 | 7.25 | 35.23 |
| Average | H | 10 | 0.9752 | 0.57087 | 58.5 | 0.8493 | 1.73518 | 0.8491 | 0.361 | 2.321 |
| Concentration | S | 11 | 1.1252 | 0.59089 | 52.5 | 1.0102 | 1.60542 | 0.9631 | 0.525 | 2.458 |
| (ng/ml) | UA | 11 | 0.9503 | 0.38077 | 40.1 | 0.8849 | 1.48608 | 0.8947 | 0.504 | 1.688 |
| | T | 11 | 0.8814 | 0.50274 | 57.0 | 0.7588 | 1.79383 | 0.7810 | 0.314 | 1.964 |
| | AB | 9 | 0.6287 | 0.22194 | 35.3 | 0.5942 | 1.43075 | 0.6296 | 0.377 | 0.988 |
| | F | 11 | 0.9607 | 0.38030 | 39.6 | 0.8846 | 1.56423 | 1.0951 | 0.389 | 1.701 |
| Time to Maximum | H | 10 | 14.75 | 9.145 | 62.0 | 17.28 | 1.553 | 18.00 | 0.0 | 23.5 |
| Concentration | S | 11 | 12.36 | 6.281 | 50.8 | 10.86 | 1.731 | 12.00 | 5.0 | 22.0 |
| (hours) | UA | 11 | 15.55 | 5.087 | 32.7 | 14.54 | 1.525 | 16.00 | 5.0 | 22.0 |
| | T | 11 | 13.82 | 7.872 | 57.0 | 16.26 | 1.366 | 16.00 | 0.0 | 22.0 |
| | AB | 9 | 13.44 | 7.468 | 55.6 | 9.54 | 3.054 | 16.00 | 1.0 | 22.0 |
| | F | 11 | 16.41 | 5.324 | 32.4 | 15.43 | 1.485 | 18.00 | 7.0 | 23.5 |
| Peak-Trough | H | 10 | 120.5 | 45.13 | 37.4 | 113.3 | 1.45 | 102.4 | 65 | 185 |
| Fluctuation (%) | S | 11 | 110.8 | 30.52 | 27.5 | 107.3 | 1.30 | 101.6 | 78 | 171 |
| | UA | 11 | 101.4 | 24.49 | 24.2 | 98.7 | 1.28 | 104.6 | 66 | 144 |
| | T | 11 | 127.6 | 78.15 | 61.2 | 111.7 | 1.67 | 90.7 | 60 | 312 |
| | AB | 9 | 100.7 | 34.78 | 34.6 | 95.5 | 1.41 | 86.2 | 59 | 154 |
| | F | 11 | 103.9 | 44.17 | 42.5 | 95.5 | 1.55 | 89.8 | 43 | 195 |
| Half Value | H | 10 | 15.06 | 6.103 | 40.5 | 13.50 | 1.740 | 15.43 | 3.8 | 23.5 |
| Duration (hour) | S | 11 | 18.34 | 4.563 | 24.9 | 17.72 | 1.339 | 20.36 | 9.6 | 22.8 |
| | UA | 11 | 18.14 | 3.700 | 20.4 | 17.76 | 1.252 | 18.46 | 10.8 | 23.2 |
| | T | 11 | 16.03 | 7.689 | 48.0 | 12.71 | 2.410 | 19.52 | 2.0 | 23.5 |
| | AB | 9 | 18.49 | 4.616 | 25.0 | 17.92 | 1.317 | 20.20 | 10.7 | 23.5 |
| | F | 11 | 16.71 | 6.323 | 37.8 | 15.37 | 1.584 | 18.78 | 5.7 | 23.5 |
| Apparent Dose of | H | 10 | 6.230 | 2.1042 | 33.8 | 5.886 | 1.4409 | 6.555 | 3.36 | 9.30 |
| Rotigotine (mg) | S | 11 | 8.157 | 1.9871 | 24.4 | 7.948 | 1.2679 | 7.480 | 5.91 | 11.20 |
| | UA | 11 | 7.034 | 1.5860 | 22.5 | 6.875 | 1.2508 | 6.920 | 4.92 | 10.21 |
| | T | 11 | 5.521 | 1.7372 | 31.5 | 5.232 | 1.4356 | 5.460 | 2.55 | 7.78 |
| | AB | 9 | 4.802 | 1.4404 | 30.0 | 4.623 | 1.3356 | 4.350 | 3.22 | 7.22 |
| | F | 11 | 7.555 | 2.0697 | 27.4 | 7.291 | 1.3270 | 8.300 | 4.63 | 11.19 |
| Days 27 and 30 | | | | | | | | | | |
| combined | | | | | | | | | | |
| AUC 0-t, ss | H | 20 | 23.815 | 12.4750 | 52.4 | 21.089 | 1.6552 | 19.491 | 8.49 | 54.54 |
| (ng * h/ml) | S | 22 | 23.794 | 12.0843 | 50.8 | 21.332 | 1.6073 | 21.304 | 7.51 | 57.77 |
| | UA | 21 | 23.053 | 10.6219 | 46.1 | 20.580 | 1.6754 | 21.025 | 5.13 | 46.50 |
| | T | 20 | 19.701 | 10.8174 | 54.9 | 16.980 | 1.7700 | 18.002 | 7.06 | 46.16 |
| | AB | 20 | 18.677 | 8.7841 | 47.0 | 17.121 | 1.5141 | 16.233 | 8.85 | 45.82 |
| | F | 23 | 24.069 | 13.8303 | 57.5 | 20.803 | 1.7470 | 23.025 | 6.73 | 68.64 |
| AUC 0-t, ss, | H | 20 | 267.69 | 123.066 | 46.0 | 245.13 | 1.522 | 229.22 | 125.6 | 581.9 |
| normalized | S | 22 | 238.82 | 87.983 | 36.8 | 222.62 | 1.486 | 249.31 | 93.3 | 421.7 |
| (ng * h * kg/ml/mg) | UA | 21 | 267.89 | 92.907 | 34.7 | 253.61 | 1.403 | 244.24 | 121.8 | 497.0 |
| | T | 20 | 251.48 | 130.002 | 51.7 | 229.27 | 1.521 | 232.36 | 118.7 | 717.8 |
| | AB | 20 | 275.15 | 178.946 | 65.0 | 242.34 | 1.617 | 232.46 | 89.3 | 952.1 |
| | F | 23 | 256.64 | 119.570 | 46.6 | 233.51 | 1.553 | 218.91 | 104.3 | 578.1 |
| Maximum | H | 20 | 1.7981 | 1.26959 | 70.6 | 1.4842 | 1.84629 | 1.3150 | 0.598 | 5.310 |
| Concentration | S | 22 | 1.5813 | 0.95110 | 60.1 | 1.3893 | 1.65227 | 1.3250 | 0.431 | 4.810 |
| (ng/ml) | UA | 21 | 1.4781 | 0.65771 | 44.5 | 1.3396 | 1.59938 | 1.2900 | 0.403 | 2.870 |
| | T | 20 | 1.3465 | 0.71031 | 52.8 | 1.1884 | 1.67848 | 1.2100 | 0.469 | 3.450 |
| | AB | 20 | 1.2936 | 0.69212 | 53.5 | 1.1563 | 1.60286 | 1.0950 | 0.502 | 3.350 |
| | F | 23 | 1.7444 | 0.93829 | 53.8 | 1.5295 | 1.69863 | 1.4300 | 0.583 | 4.610 |
| Maximum Conc | H | 20 | 19.956 | 13.2635 | 66.5 | 17.253 | 1.6685 | 15.577 | 9.46 | 61.31 |
| normalized | S | 22 | 15.898 | 7.0630 | 44.4 | 14.499 | 1.5627 | 15.328 | 5.60 | 35.11 |
| (ng * kg/ml/mg) | UA | 21 | 17.727 | 7.3798 | 41.6 | 16.508 | 1.4597 | 15.499 | 7.68 | 39.01 |
| | T | 20 | 17.793 | 9.8823 | 55.5 | 16.046 | 1.5548 | 15.506 | 7.69 | 53.65 |
| | AB | 20 | 18.470 | 10.2802 | 55.7 | 16.367 | 1.6473 | 14.906 | 4.96 | 52.52 |
| | F | 23 | 18.976 | 8.6532 | 45.6 | 17.168 | 1.5894 | 16.666 | 7.25 | 38.83 |
| Average | H | 20 | 1.0134 | 0.53085 | 52.4 | 0.8974 | 1.65522 | 0.8294 | 0.361 | 2.321 |
| Concentration | S | 22 | 1.0125 | 0.51423 | 50.8 | 0.9077 | 1.60729 | 0.9065 | 0.319 | 2.458 |
| (ng/ml) | UA | 21 | 0.9810 | 0.45199 | 46.1 | 0.8758 | 1.67538 | 0.8947 | 0.218 | 1.979 |
| | T | 20 | 0.8383 | 0.46031 | 54.9 | 0.7226 | 1.76997 | 0.7660 | 0.300 | 1.964 |
| | AB | 20 | 0.7947 | 0.37379 | 47.0 | 0.7286 | 1.51410 | 0.6908 | 0.377 | 1.950 |
| | F | 23 | 1.0242 | 0.58853 | 57.5 | 0.8853 | 1.74705 | 0.9798 | 0.286 | 2.921 |
| Time to Maximum | H | 20 | 14.13 | 8.114 | 57.4 | 15.12 | 1.666 | 17.00 | 0.0 | 23.5 |
| Concentration | S | 22 | 12.91 | 6.879 | 53.3 | 12.80 | 1.653 | 15.00 | 0.0 | 22.0 |
| (hours) | UA | 21 | 12.71 | 7.830 | 61.6 | 14.69 | 1.504 | 14.00 | 0.0 | 22.0 |
| | T | 20 | 13.00 | 7.719 | 59.4 | 15.50 | 1.417 | 16.00 | 0.0 | 22.0 |
| | AB | 20 | 13.18 | 7.576 | 57.5 | 10.68 | 2.514 | 16.00 | 0.0 | 23.5 |
| | F | 23 | 15.00 | 7.145 | 47.6 | 13.40 | 2.010 | 16.00 | 0.0 | 23.5 |
| Peak-Trough | H | 20 | 117.4 | 43.10 | 36.7 | 110.3 | 1.44 | 102.5 | 61 | 198 |
| Fluctuation (%) | S | 22 | 105.2 | 26.22 | 24.9 | 102.3 | 1.27 | 102.2 | 72 | 171 |
| | UA | 21 | 98.1 | 28.89 | 29.5 | 94.1 | 1.35 | 93.8 | 51 | 168 |
| | T | 20 | 112.9 | 61.12 | 54.1 | 102.4 | 1.53 | 98.5 | 57 | 312 |
| | AB | 20 | 109.4 | 56.21 | 51.4 | 100.4 | 1.48 | 88.0 | 59 | 314 |
| | F | 23 | 127.0 | 45.34 | 35.7 | 118.4 | 1.49 | 123.5 | 43 | 200 |
| Half Value | H | 20 | 15.73 | 6.132 | 39.0 | 14.17 | 1.685 | 15.95 | 3.8 | 23.5 |
| Duration (hour) | S | 22 | 18.35 | 4.292 | 23.4 | 17.79 | 1.305 | 20.33 | 9.6 | 23.0 |
| | UA | 21 | 18.71 | 3.705 | 19.8 | 18.33 | 1.239 | 18.64 | 10.8 | 23.5 |
| | T | 20 | 17.11 | 6.192 | 36.2 | 14.90 | 1.965 | 19.23 | 2.0 | 23.5 |
| | AB | 20 | 18.18 | 5.469 | 30.1 | 16.28 | 1.912 | 20.33 | 1.2 | 23.5 |
| | F | 23 | 13.95 | 5.959 | 42.7 | 12.64 | 1.604 | 12.47 | 4.5 | 23.5 |
| Apparent Dose of | H | 20 | 6.546 | 2.0483 | 31.3 | 6.228 | 1.3916 | 6.455 | 3.36 | 10.33 |
| Rotigotine (mg) | S | 22 | 7.315 | 2.1468 | 29.3 | 6.994 | 1.3721 | 7.270 | 3.53 | 11.20 |
| | UA | 21 | 6.360 | 2.2749 | 35.8 | 5.808 | 1.6512 | 6.360 | 1.06 | 10.21 |
| | T | 20 | 5.934 | 2.3312 | 39.3 | 5.528 | 1.4728 | 5.245 | 2.55 | 11.57 |
| | AB | 20 | 5.664 | 2.1830 | 38.5 | 5.223 | 1.5453 | 5.000 | 1.61 | 9.38 |
| | F | 23 | 7.400 | 2.3565 | 31.8 | 7.075 | 1.3521 | 7.090 | 4.63 | 13.52 |

Table 18 shows the summary statistics for AUC_{O-t,ss} and C_{max,ss} for unconjugated rotigotine for each site combined data from Day 27 and Day 30.

TABLE 18
Summary statistics of derived PK parameters for area under the curve
(AUC_{0-t,ss,normalized}) and maximum plasma concentration (C_{max,normalized}) of
unconjugated rotigotine for each patch application site after normalization for
body weight and apparent dose, data from Day 27 and Day 30 combined (PKS)

| | | | | | | |
|---|---|---|---|---|---|---|
| Application | | | | Geometric mean | | |
| Site | n | Mean (SD) | CV (%) | (SD) | Median | Range |

AUC_{0-t,ss,normalized} (ng * h * kg/mL/mg)

| | | | | | | |
|---|---|---|---|---|---|---|
| Hip | 20 | 267.69 | 46.0 | 245.13 (1.522) | 229.22 | 125.6-581.9 |
| | | (123.066) | | | | |
| Shoulder | 22 | 238.82 | 36.8 | 222.62 (1.486) | 249.31 | 93.3-421.7 |
| | | (87.983) | | | | |
| Upper arm | 21 | 267.89 | 34.7 | 253.61 (1.403) | 244.24 | 121.8-497.0 |
| | | (92.907) | | | | |
| Thigh | 20 | 251.48 | 51.7 | 229.27 (1.521) | 232.36 | 118.7-717.8 |
| | | (130.002) | | | | |
| Abdomen | 20 | 275.15 | 65.0 | 242.34 (1.617) | 232.46 | 89.3-952.1 |
| | | (178.946) | | | | |
| Flank | 23 | 256.64 | 46.6 | 233.51 (1.553) | 218.91 | 104.3-578.1 |
| | | (119.570) | | | | |

C_{max,ss, normalized} (ng * kg/mL/mg)

| | | | | | | |
|---|---|---|---|---|---|---|
| Hip | 20 | 19.956 | 66.5 | 17.253 (1.6685) | 15.577 | 9.46-61.31 |
| | | (13.2635) | | | | |
| Shoulder | 22 | 15.898 | 44.4 | 14.499 (1.5627) | 15.328 | 5.60-35.11 |
| | | (7.0630) | | | | |
| Upper arm | 21 | 17.727 | 41.6 | 16.508 (1.4597) | 15.499 | 7.68-39.01 |
| | | (7.3798) | | | | |
| Thigh | 20 | 17.793 | 55.5 | 16.046 (1.5548) | 15.506 | 7.69-53.65 |
| | | (9.8823) | | | | |
| Abdomen | 20 | 18.470 | 55.7 | 16.367 (1.6473) | 14.906 | 4.96-52.52 |
| | | (10.2802) | | | | |
| Flank | 23 | 18.976 | 45.6 | 17.168 (1.5894) | 16.666 | 7.25-38.83 |
| | | (8.6532) | | | | |

PKS = pharmacokinetic set;
SD = standard deviation;
CV = coefficient of variance

### EXAMPLE 5

A multicenter, randomized, double-blind, placebo-controlled, 2-arm, parallel group clinical trial was performed to evaluate the safety and efficacy of a rotigotine patch in subjects with early stage, idiopathic Parkinson's disease. The silicone transdermal patches were made in accordance with the teachings of U.S. Patent Application Publication No. US 2003/0026830 at paragraphs 38-42, U.S. Patent Application Publication No. US 2003/0027793 at paragraphs 3741 and U.S. Pat. No. 6,884,434, columns 5-6, Example 2 and comprised the following layers and components:

Patches D, E and F

| | Patch D (mg/10 | | Patch E (mg/20 | | Patch F (mg/30 | |
|---|---|---|---|---|---|---|
| Name of Ingredient | cm² patch) | | cm² patch) | | cm² patch) | |
| Rotigotine | 4.50 | | 9.00 | | 13.50 | |
| Silicone adhesive 4301 | 22.24 | | 44.47 | | 66.71 | |
| Silicone adhesive 4201 | 22.23 | | 44.46 | | 66.70 | |
| Providone | 1.00 | | 2.00 | | 3.00 | |
| Sodium metabisulfite | 0.00045 | | 0.0009 | | 0.00135 | |
| Ascorbyl palmitate | 0.010 | | 0.02 | | 0.03 | |
| Vitamin E | 0.025 | | 0.05 | | 0.075 | |
| (DL-α-tocopherol) | | | | | | |
| Backing foil PET, | 10 | cm² | 20 | cm² | 30 | cm² |
| siliconized aluminized, | | | | | | |
| color coated | | | | | | |

| | | | |
|---|---|---|---|
| Ink Bargofor | As much as | As much as | As much as |
| 70135-1-P | needed | needed | needed |

The doses included 4.5 mg/day, 9 mg/day, and 13.5 mg/day of rotigotine. Trial periods consisted of a 4-week pre-treatment (washout) period, a 3-week dose escalation period, a 25-week dose maintenance period, and a 4-week follow-up period for a total duration of 36 weeks.

Plasma samples for measurement of rotigotine concentration were collected in 56 subjects. The total number of samples was 1297. During the study blood samples for the analysis of rotigotine were taken before patch application and at 1, 2, 3, 11, 19, and 28 weeks after first patch application.

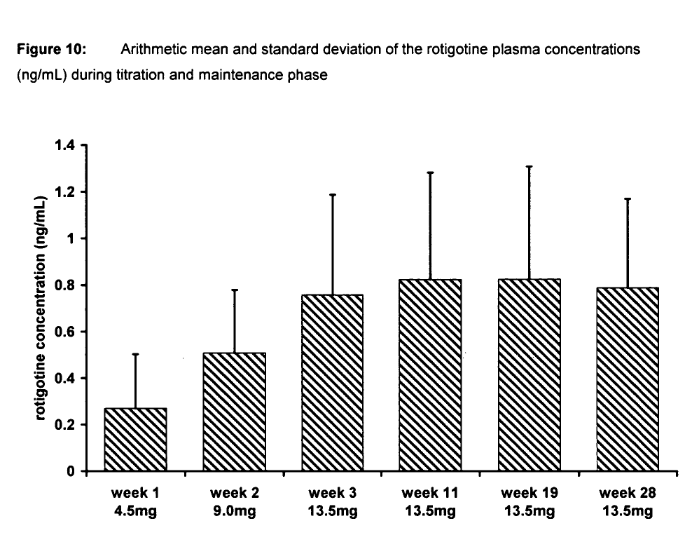

Table 19 shows the results of descriptive statistics for concentrations of rotigotine in plasma samples. FIG. 10 illustrates the results. This figure shows stable concentration over the maintenance phase of the study.

TABLE 19
Descriptive statistics of rotigotine plasma concentrations (ng/mL)
during titration and maintenance phase

| Day Period | Dose (mg/day) | Sampling Time | n | Mean | SD | Median | Min | Max |
|---|---|---|---|---|---|---|---|---|
| Day 8 TP | 4.5 | Prior removal | 54 | 0.270 | 0.234 | 0.222 | 0.024 | 1.670 |
| Day 15 TP | 9.0 | Prior removal | 51 | 0.508 | 0.272 | 0.435 | 0.053 | 1.580 |
| Day 1 MP | 13.5 | Prior removal | 48 | 0.757 | 0.430 | 0.714 | 0.064 | 2.130 |
| Day 57 MP | 13.5 | Prior removal | 45 | 0.824 | 0.459 | 0.702 | 0.103 | 2.070 |
| Day 113 MP | 13.5 | Prior removal | 41 | 0.825 | 0.483 | 0.713 | 0.122 | 2.420 |
| End of MP | 13.5 | Prior removal | 39 | 0.788 | 0.382 | 0.729 | 0.282 | 1.800 |

Min = minimum;
Max = maximum;
MP = maintenance period;
SD = standard deviation
TP = titration period.

What is claimed is

1. A method for treating Parkinson's Disease in a human patient, comprising administering to the patient a rotigotine formulation capable of providing a plasma concentration effective to alleviate the symptoms of Parkinson's Disease, wherein the Cₘₐₓ is from about 0.14 ng/mL to about 1.54 ng/mL and wherein the mean area under the curve (AUCₒ₋ₜ) is from about 3.3 ng/mL *h to about 32.2 ng/mL *h.

2. The method of claim 1, wherein the rotigotine is transdermally administered.

3. The method of claim 1, wherein the Cₘₐₓ is from about about 0.20 ng/mL to about 1.30 ng/mL.

4. The method of claim 1, wherein the Cₘₐₓ is from about 0.30 ng/mL to about 1.20 ng/mL.

5. The method of claim 1, wherein the Cₘₐₓ is from about 0.14 ng/mL to about 0.48 ng/mL.

6. The method of claim 1, wherein the Cₘₐₓ is from about 0.37 ng/mL to about 0.75 ng/mL.

7. The method of claim 1, wherein the Cₘₐₓ is from about 0.84 ng/mL to about 1.54 ng/mL.

8. The method of claim 1, wherein the Cₘₐₓ is about 0.31 ng/mL.

9. The method of claim 1, wherein the Cₘₐₓ is about 0.56 ng/mL.

10. The method of claim 1, wherein the Cₘₐₓ is about 1.19 ng/mL.

11. The method of claim 1, wherein the AUC_{O-t} is from about 4.0 ng/mL *h to about 30.0 ng/mL *h.

12. The method of claim 1, wherein the AUC_{O-t} is from about 5.0 ng/mL *h to about 25.0 ng/mL *h.

13. The method of claim 1, wherein the AUC_{O-t} is from about 3.3 ng/mL *h to about 8.9 ng/mL *h.

14. The method of claim 1, wherein the AUC_{O-t} is from about 7 ng/mL *h to about 15.2 ng/mL *h.

15. The method of claim 1, wherein the AUC_{O-t} is from about 15.2 ng/mL *h to about 32.2 ng/mL *h.

16. The method of claim 1, wherein the AUC_{O-t} is about 6.1 ng/mL *h.

17. The method of claim 1, wherein the AUC_{O-t} is about 11.1 ng/mL *h.

18. The method of claim 1, wherein the AUC_{O-t} is about 23.7 ng/mL *h.

19. The method of claim 1, wherein the method provides the plasma concentration effective to alleviate the symptoms of Parkinson's disease regardless of where the rotigotine is administered to the body of the human patient.

20. A method for treating Parkinson's Disease in a human patient, comprising administering to the patient a rotigotine formulation capable of maintaining a plasma concentration effective to alleviate the symptoms of Parkinson's Disease, wherein the Cₘₐₓ is sustained at a level from about 0.14 ng/mL to about 1.54 ng/mL and wherein the mean area under the curve (AUCₒ₋ₜ) is from about 3.3 ng/mL *h to about 32.2 ng/mL *h.

21. The method of claim 20, wherein the rotigotine is transdermally administered.

22. The method of claim 20, wherein the Cₘₐₓ is from about about 0.20 ng/mL to about 1.30 ng/mL.

23. The method of claim 20, wherein the Cₘₐₓ is from about 0.30 ng/mL to about 1.20 ng/mL.

24. The method of claim 20, wherein the Cₘₐₓ is from about 0.14 ng/mL to about 0.48 ng/mL.

25. The method of claim 20, wherein the Cₘₐₓ is from about 0.37 ng/mL to about 0.75 ng/mL.

26. The method of claim 20, wherein the Cₘₐₓ is from about 0.84 ng/mL to about 1.54 ng/mL.

27. The method of claim 20, wherein the Cₘₐₓ is about 0.31 ng/mL.

28. The method of claim 20, wherein the Cₘₐₓ is about 0.56 ng/mL.

29. The method of claim 20, wherein the Cₘₐₓ is about 1.19 ng/mL.

30. The method of claim 20, wherein the AUC_{O-t} is from about 4.0 ng/mL *h to about 30.0 ng/mL *h.

31. The method of claim 20, wherein the AUC_{O-t} is from about 5.0 ng/mL *h to about 25.0 ng/mL *h.

32. The method of claim 20, wherein the AUC_{O-t} is from about 3.3 ng/mL *h to about 8.9 ng/mL *h.

33. The method of claim 20, wherein the AUC_{O-t} is from about 7 ng/mL *h to about 15.2 ng/mL *h.

34. The method of claim 20, wherein the AUC_{O-t} is from about 15.2 ng/mL *h to about 32.2 ng/mL *h.

35. The method of claim 20, wherein the AUC_{O-t} is about 6.1 ng/mL *h.

36. The method of claim 20, wherein the AUC_{O-t} is about 11.1 ng/mL *h.

37. The method of claim 20, wherein the AUC_{O-t} is about 23.7 ng/mL *h.

38. The method of claim 20, wherein the method provides the plasma concentration effective to alleviate the symptoms of Parkinson's disease regardless of where the rotigotine is administered to the body of the human patient.

39. A method of treating Parkinson's Disease in a human patient, comprising applying a transdermal therapeutic system (TTS) comprising rotigotine, wherein the TTS is capable of providing a plasma concentration effective to alleviate the symptoms of Parkinson's Disease, wherein the Cₘₐₓ is from about 0.14 ng/mL to about 1.54 ng/mL and wherein the mean area under the curve (AUCₒ₋ₜ) is from about 3.3 ng/mL *h to about 32.2 ng/mL *h.

40. The method of claim 39, wherein the Cₘₐₓ is from about about 0.20 ng/mL to about 1.30 ng/mL.

41. The method of claim 39, wherein the Cₘₐₓ is from about 0.30 ng/mL to about 1.20 ng/mL.

42. The method of claim 39, wherein the Cₘₐₓ is from about 0.14 ng/mL to about 0.48 ng/mL.

43. The method of claim 39, wherein the Cₘₐₓ is from about 0.37 ng/mL to about 0.75 ng/mL.

44. The method of claim 39, wherein the Cₘₐₓ is from about 0.84 ng/mL to about 1.54 ng/mL.

45. The method of claim 39, wherein the Cₘₐₓ is about 0.31 ng/mL.

46. The method of claim 39, wherein the Cₘₐₓ is about 0.56 ng/mL.

47. The method of claim 39, wherein the Cₘₐₓ is about 1.19 ng/mL.

48. The method of claim 39, wherein the AUC_{O-t} is from about 4.0 ng/mL *h to about 30.0 ng/mL *h.

49. The method of claim 39, wherein the AUC_{O-t} is from about 5.0 ng/mL *h to about 25.0 ng/mL *h.

50. The method of claim 39, wherein the AUC_{O-t} is from about 3.3 ng/mL *h to about 8.9 ng/mL *h.

51. The method of claim 39, wherein the AUC_{O-t} is from about 7 ng/mL *h to about 15.2 ng/mL *h.

52. The method of claim 39, wherein the AUC_{O-t} is from about 15.2 ng/mL *h to about 32.2 ng/mL *h.

53. The method of claim 39, wherein the AUC_{O-t} is about 6.1 ng/mL *h.

54. The method of claim 39, wherein the AUC_{O-t} is about 11.1 ng/mL *h.

55. The method of claim 39, wherein the AUC_{O-t} is about 23.7 ng/mL *h.

56. The method of claim 39, wherein the method provides the plasma concentration effective to alleviate the symptoms of Parkinson's disease regardless of where the rotigotine is administered to the body of the human patient.

57. A method for treating Parkinson's Disease in a human patient, comprising administering to the patient over a 24 hr period a rotigotine formulation capable of providing a plasma concentration effective to alleviate the symptoms of Parkinson's Disease, wherein the Cₘₐₓ is from about 0.14 ng/mL to about 1.54 ng/mL and the AUC_{O-t} is from about 3.3 ng/mL *h to about 32.2 ng/mL *h.

58. The method of claim 57, wherein the rotigotine is transdermally administered.

59. The method of claim 57, wherein the Cₘₐₓ is from about about 0.20 ng/mL to about 1.30 ng/mL.

60. The method of claim 57, wherein the Cₘₐₓ is from about 0.30 ng/mL to about 1.20 ng/mL.

61. The method of claim 57, wherein the Cₘₐₓ is from about 0.14 ng/mL to about 0.48 ng/mL.

62. The method of claim 57, wherein the Cₘₐₓ is from about 0.37 ng/mL to about 0.75 ng/mL.

63. The method of claim 57, wherein the Cₘₐₓ is from about 0.84 ng/mL to about 1.54 ng/mL.

64. The method of claim 57, wherein the Cₘₐₓ is about 0.31 ng/mL.

65. The method of claim 57, wherein the Cₘₐₓ is about 0.56 ng/mL.

66. The method of claim 57, wherein the Cₘₐₓ is about 1.19 ng/mL.

67. The method of claim 57, wherein the AUC_{O-t} is from about 4.0 ng/mL *h to about 30.0 ng/mL *h.

68. The method of claim 57, wherein the AUC_{O-t} is from about 5.0 ng/mL *h to about 25.0 ng/mL *h.

69. The method of claim 57, wherein the AUC_{O-t} is from about 3.3 ng/mL *h to about 8.9 ng/mL *h.

70. The method of claim 57, wherein the AUC_{O-t} is from about 7 ng/mL *h to about 15.2 ng/mL *h.

71. The method of claim 57, wherein the AUC_{O-t} is from about 15.2 ng/mL *h to about 32.2 ng/mL *h.

72. The method of claim 57, wherein the AUC_{O-t} is about 6.1 ng/mL *h.

73. The method of claim 57, wherein the AUC_{O-t} is about 11.1 ng/mL *h.

74. The method of claim 57, wherein the AUC_{O-t} is about 23.7 ng/mL *h.

75. The method of claim 57, wherein the method provides the plasma concentration effective to alleviate the symptoms of Parkinson's disease regardless of where the rotigotine is administered to the body of the human patient.

76. A method of treating Parkinson's Disease in a human patient comprising applying one or more transdermal patches comprising an amount of rotigotine from 4 mg to 20 mg to the patient to provide a plasma concentration effective to alleviate the symptoms of Parkinson's Disease in the human patient, wherein the Cₘₐₓ is sustained at a level from about 0.14 ng/mL to about 1.54 ng/mL and the mean area under the curve (AUCₒ₋ₜ) in the patient is from about 3.3 ng/mL *h to about 32.2 ng/mL *h.

77. The method of claim 76, wherein the Cₘₐₓ is from about 0.20 ng/mL to about 1.30 ng/mL.

78. The method of claim 76, wherein the Cₘₐₓ is from about 0.30 ng/mL to about 1.20 ng/mL.

79. The method of claim 76, wherein the Cₘₐₓ is from about 0.14 ng/mL to about 0.48 ng/mL.

80. The method of claim 76, wherein the Cₘₐₓ is from about 0.37 ng/mL to about 0.75 ng/mL.

81. The method of claim 76, wherein the Cₘₐₓ is from about 0.84 ng/mL to about 1.54 ng/mL.

82. The method of claim 76, wherein the Cₘₐₓ is about 0.31 ng/mL.

83. The method of claim 76, wherein the Cₘₐₓ is about 0.56 ng/mL.

84. The method of claim 76, wherein the Cₘₐₓ is about 1.19 ng/mL.

85. The method of claim 76, wherein the AUC_{O-t} is from about 4.0 ng/mL *h to about 30.0 ng/mL *h.

86. The method of claim 76, wherein the AUC_{O-t} is from about 5.0 ng/mL *h to about 25.0 ng/mL *h.

87. The method of claim 76, wherein the AUC_{O-t} is from about 3.3 ng/mL *h to about 8.9 ng/mL *h.

88. The method of claim 76, wherein the AUC_{O-t} is from about 7 ng/mL *h to about 15.2 ng/mL *h.

89. The method of claim 76, wherein the AUC_{O-t} is from about 15.2 ng/mL *h to about 32.2 ng/mL *h.

90. The method of claim 76, wherein the AUC_{O-t} is about 6.1 ng/mL *h.

91. The method of claim 76, wherein the AUC_{O-t} is about 11.1 ng/mL *h.

92. The method of claim 76, wherein the AUC_{O-t} is about 23.7 ng/mL *h.

93. The method of claim 76, wherein the method provides the plasma concentration effective to alleviate the symptoms of Parkinson's disease regardless of where the rotigotine is administered to the body of the human patient.

94. A method of treating Parkinson's Disease in a human patient comprising
a) applying one or more transdermal patches comprising an amount of rotigotine from 4 mg to 20 mg to the patient;
b) removing the patch or patches of step a) and applying another patch or patches comprising an amount of rotigotine from 4 mg to 20 mg to the patient at an interval providing a plasma concentration effective to alleviate the symptoms of Parkinson's Disease in the patient; and
c) repeating step b) as required to sustain the Cₘₐₓ at a level effective to alleviate the symptoms of Parkinson's Disease in the human patient,
wherein the Cₘₐₓ is sustained at a level from about 0.14 ng/mL to about 1. 54 ng/mL.

95. The method of claim 94, wherein the Cₘₐₓ is from about 0.20 ng/mL to about 1.30 ng/mL.

96. The method of claim 94, wherein the Cₘₐₓ is from about 0.30 ng/mL to about 1.20 ng/mL.

97. The method of claim 94, wherein the Cₘₐₓ is from about 0.14 ng/mL to about 0.48 ng/mL.

98. The method of claim 94, wherein the Cₘₐₓ is from about 0.37 ng/mL to about 0.75 ng/mL.

99. The method of claim 94, wherein the Cₘₐₓ is from about 0.84 ng/mL to about 1.54 ng/mL.

100. The method of claim 94, wherein the Cₘₐₓ is about 0.31 ng/mL.

101. The method of claim 94, wherein the Cₘₐₓ is about 0.56 ng/mL.

102. The method of claim 94, wherein the Cₘₐₓ is about 1.19 ng/mL.

103. The method of claim 94, wherein the Cₘₐₓ is sustained at a level effective to alleviate the symptoms of Parkinson's Disease in the human patient for a period from 1 day to 7 days.

104. The method of claim 94, wherein the Cₘₐₓ is sustained at a level effective to alleviate the symptoms of Parkinson's Disease in the human patient for a period of time is from 1 week to 6 weeks.

105. The method of claim 94, wherein the Cₘₐₓ is sustained at a level effective to alleviate the symptoms of Parkinson's Disease in the human patient for a period of time is 7 weeks.

106. The method of claim 94, wherein the Cₘₐₓ is sustained at a level effective to alleviate the symptoms of Parkinson's Disease in the human patient for a period of time is from 8 weeks to 28 weeks.

107. The method of claim 94, wherein the method gives the plasma concentration effective to alleviate the symptoms of Parkinson's Disease regardless of where the patches are applied on the body of the human patient.

108. The method of claim 94, wherein the patch or patches are replaced daily.

109. The method of claim 94, wherein the Cₘₐₓ is sustained at a level effective to alleviate the symptoms of Parkinson's Disease in the human patient for a period of time from 1 day to 28 weeks.

110. A method for treating Parkinson's Disease in a human patient, comprising administering over a 24 hour period a rotigotine formulation capable of providing a plasma concentration effective to alleviate the symptoms of Parkinson's Disease, wherein the Cₘₐₓ is from about 0.14 ng/mL to about 1.54 ng/mL and the AUC_{O-t} is from about 3.3 ng/mL *h to about 32.2 ng/mL *h, regardless of where the rotigotine is administered to the body of the human patient.

111. A controlled release rotigotine formulation for transdermal administration to a human patient, comprising from about 4 mg to about 20 mg of rotigotine, wherein the formulation provides a plasma concentration wherein the (Cₘₐₓ) of rotigotine is from about 0.14 ng/mL to about 1.54 ng/mL and a mean area under the curve up to the last quantifiable concentration (AUCₒ₋ₜ) from about 3.3 ng/mL *h to about 32.2 ng/mL *h, regardless of where the rotigotine formulation is applied on the body of the human patient.

112. The formulation of claim 111, wherein said human patient is suffering from Parkinson's Disease.

113. The formulation of claim 111 wherein the formulation is present as a Transdermal Therapeutic System (TTS) consisting of a protective liner, a self-adhesive drug matrix layer consisting of at least the active component rotigotine and a flexible backing which is preferably siliconised on its inner side and is consisting of an aluminized polyester foil coated with a pigment layer on the outer side.

114. The formulation of claim 111 wherein the flexible backing is a transparent polyester film

115. A method for inducing treating Restless Legs Syndrome in a human patient, comprising administering a rotigotine formulation capable of providing a plasma concentration effective to alleviate symptoms of Restless Legs Syndrome, wherein the Cₘₐₓ is from about 0.14 ng/mL to about 1.54 ng/mL and the AUC_{O-t} is from about 3.3 ng/mL *h to about 32.2 ng/mL *h.

116. A method for treating Restless Legs Syndrome in a human patient, comprising administering to the patient over a 24 hr period a rotigotine formulation capable of providing a plasma concentration effective to alleviate the symptoms of Restless Legs Syndrome, wherein the Cₘₐₓ is from about 0.14 ng/mL to about 1.54 ng/mL and the AUC_{O-t} is from about 3.3 ng/mL *h to about 32.2 ng/mL *h.

117. The method of claim 116, wherein the method provides the plasma concentration effective to alleviate the symptoms of Restless Legs Syndrome regardless of where the rotigotine is administered to the body of the human patient.

118. A method for treating a disease related to the dopaminergic system in a human patient, comprising administering to the patient over a 24 hr period a rotigotine formulation capable of providing a plasma concentration effective to alleviate the symptoms of the disease related to the dopaminergic system, wherein the Cₘₐₓ is from about 0.14 ng/mL to about 1.54 ng/mL and the AUC_{O-t} is from about 3.3 ng/mL *h to about 32.2 ng/mL *h.

119. A method for treating a disease related to the dopaminergic system in a human patient, comprising administering rotigotine to the patient wherein the Cₘₐₓ is from about 0.14 ng/mL to about 1.54 ng/mL and wherein the AUC_{O-t} is from about 3.3 ng/mL *h to about 32.2 ng/mL *h.

120. The method of claim 119, wherein the method provides the plasma concentration effective to alleviate the symptoms of the disease related to the dopaminergic system regardless of where the rotigotine is administered to the body of the human patient.
